(19) United States
(12) Patent Application Publication
(10) Pub. No.: US 2021/0369615 A1
(43) Pub. Date: Dec. 2, 2021

(54) SOLID ORAL FORMULATIONS OF AMPHOTERICIN B

(71) Applicant: iCo Therapeutics Inc., Vancouver (CA)

(72) Inventors: Peter HNIK, Vancouver, BC (CA); Roch THIBERT, Quebec (CA); Aimesther BETANCOURT, Quebec (CA)

(21) Appl. No.: 17/264,046

(22) PCT Filed: Jul. 31, 2019
(86) PCT No.: PCT/US2019/044377

§ 371 Date: Jan. 28, 2021

Related U.S. Application Data
(60) Provisional application No. 62/712,593, filed on Jul. 31, 2018.

(51) Int. Cl. A61K 9/16; A61K 31/7048; A61K 9/48

(52) U.S. Cl. CPC A61K 9/1676; A61K 31/7048; A61K 9/4858; A61K 9/4866; A61K 9/1617

(57) ABSTRACT

The present disclosure describes methods of treating infectious diseases with solid dosage forms comprising amphotericin B. In some embodiments, the disclosure provides methods of treating fungal infections and Lesishmania infection.

### CROSS REFERENCE TO RELATED APPLICATIONS

This application claims the benefit of priority of U.S. Provisional Application No. 62/712,593, filed Jul. 31, 2018.

### BACKGROUND

Amphotericin B is an effective antifungal agent and is the drug of choice for treating serious systemic fungal infections and Lesishmania infections. However, amphotericin B has several unfavorable properties which severely impede its use as a therapeutic agent. First, amphotericin B is insoluble in water. Second, amphotericin B cannot be absorbed in the gastrointestinal tract (GIT). Third, amphotericin B is not stable in the acid environment of the stomach. Each of these properties limits the bioavailability of amphotericin B.

To overcome the above problems which result in limited bioavailability, amphotericin B was administered in a liposomal composition (Ampbisome®) or as colloidal dispersion (Fungizone®, Abelcet®). However, intravenous injection and infusion of amphotericin B have significant disadvantages. First, the intravenous injection and infusion of amphotericin B have been associated with considerable side effects such as fever, chills, bone pain, nephrotoxicity, and thrombophlebitis. Second, intravenous amphotericin B must be administered over 30-40 days, and thus this dosing regimen is expensive and suffers from low patient compliance. These drawbacks are particularly issues in developing countries where Lesishmania infections occur.

U.S. Pat. Nos. 8,592,382 and 8,673,866 describe orally administered liquid formulations comprising amphotericin B and a mixture of fatty acid glycerol esters and polyethylene oxide-containing fatty acid esters. The fatty acid glycerol esters and polyethylene oxide-containing fatty acid esters are present in substantial excess (greater than 180:1) relative to amphotericin B, which was described as critical to achieving bioavailability of amphotericin B in an oral dosage form. However, the large amount of oily components in these formulations may cause gastric upset, such as nausea and diarrhea which limits patient compliance, particularly since an extended dosing regime is required. In addition, dosing such liquid suspensions is messy and can result in under or overdosing due to dispensing errors, spillage, and/or losses of residual formulation remaining in the dispensing device. There is thus a need to provide stable bioavailable dosage forms of amphotericin B, ideally solid dosage forms, which do not exhibit the limitations of known amphotericin B formulations.

The present disclosure provides a solid dosage form which overcomes the limitations of the conventional amphotericin B compositions.

### SUMMARY

The disclosure, in various embodiments, is directed to solid dosage forms (e.g., solid or semi-solid dosage forms) comprising lipophilic drugs, for example amphotericin B. In embodiments, the solid dosage forms disclosed herein achieve bioavailability equivalent to liquid formulations commonly used to administer amphotericin B.

In some embodiments, the solid dosage form comprises amphotericin B and at least one lipophilic component which are coated on a solid carrier. In other embodiments, the % w/w of amphotericin B in the solid dosage form is greater than a % w/w of the at least one lipophilic component. In further embodiments, the % w/w of amphotericin B is in the range of about 20% to about 30% of the total weight of the solid dosage form.

In some embodiments, amphotericin B is present in the solid dosage form in a therapeutically effective amount. In other embodiments, amphotericin B is present in amount in the range of from about 100 mg to about 800 mg, e.g., about 100 mg, about 150 mg, about 200 mg, about 250 mg, about 300 mg, about 350 mg, about 400 mg, about 450 mg, about 500 mg, about 550, about 600 mg, about 650 mg, about 700 mg, about 750 mg, and about 800 mg.

In some embodiments, the at least one lipophilic component is selected from the group consisting of a polyethylene oxide-containing fatty acid ester, fatty acid glycerol ester, and a combination thereof.

In some embodiments, the solid carrier is a bead or a saccharide. In other embodiments, the disclosure provides for a capsule comprising a solid dosage form described herein.

In some embodiments, the solid dosage forms disclosed herein comprise: a) amphotericin B in an amount from about 100 to about 800 mg; b) at least one lipophilic component, wherein the amphoteric B and the at least one lipophilic component are coated on a solid carrier; and wherein the solid dosage form provides at least one of the following pharmacokinetic parameters: i) an average maximum blood plasma concentration (Cmax) of amphotericin B of about 80%-125% of the range of about 21.09 ng/mL to about 42.07 ng/mL, after a single dose of about 100-800 mg of amphotericin B; ii) an average time to Cₘₐₓ (Tₘₐₓ) of about 80%-125% of the range of about 5.25 hr to about 9.66 hr after a single dose of about 100-400 mg of amphotericin B; iii) an average AUC₀₋ₜ within about 80%-125% of the range of from about 510.00 hr*ng/mL to about 4779.45.89 hr*ng/mL after a single dose of about 100-800 mg of amphotericin B; and iv) an average AUC_{0-inf} within about 80%-125% of the range of about 509.84 18366.24 hr*ng/mL to about 18366.24 hr*ng/mL after a single dose of about 100 to about 400 mg of amphotericin B.

In some embodiments, the present disclosure provides for a method of treating a disease in a subject in need thereof, comprising administering a solid dosage form of the present disclosure comprising: a) about 100-800 mg of amphotericin B; and b) at least one lipophilic component, wherein the amphoteric B and the at least one lipophilic component are coated on a solid carrier; and wherein after administration, the patient has at least one of the following pharmacokinetic parameters: i) an average Cₘₐₓ of amphotericin B of about 80%-125% of the range of about 21.09 ng/mL to about 42.07 ng/mL, after a single dose of about 100-800 mg of amphotericin B; ii) an average mean time to Tₘₐₓ of about 80%-125% of the range of about 5.25 hr to about 9.66 hr after a single dose of about 100-800 mg of amphotericin B; iii) an average AUC₀₋ₜ within 80%-125% of the range of from about 551.86 hr*ng/mL to about 1654.89 hr*ng/mL after a single dose of about 100-800 mg of amphotericin B; and iv) an average AUG_{0-inf} within 80%-125% of the range of about 18366.24 hr*ng/mL to about 18366.24 hr*ng/mL after a single dose of about 100-800 mg of amphotericin B.

In some embodiments, the disclosure provides for a method of treating a disease in a subject in need thereof, comprising administering amphotericin B in an amount in the range of from 100-800 mg, wherein after administration, the patient has an average AUC₀₋ₜ within 80%-125% of the range of from about 551.86 hr*ng/mL to about 1654.89 hr*ng/mL, or an average AUC_{0-inf} within 80%-125% of the range of 18366.24 hr*ng/mL to about 18366.24 hr*ng/mL after a single dose of about 100-800 mg of amphotericin B.

In some embodiments, the subject is human. In some embodiments, subject is treated for an infectious disease. In some embodiments, the infectious disease is a fungal infection, human immunodeficiency virus (HIV), or a parasitic infection. In some embodiments, the fungal infection is aspergillosis, blastomycosis, candidiasis, coccidioidomycosis, crytococcosis, histoplasmosis, mucormycosis, paracoccidioidomycosis, or sporotrichosis. In some embodiments, the parasitic infection is visceral leishmaniasis, cutaneous leishmaniasis, mucocutaneous leishmaniasis, or Chagas disease. In some embodiments, the infectious disease is leishmaniasis. In some embodiments, the infectious disease is Febrile neutropenia.

### DESCRIPTION OF THE DRAWINGS

FIG. 1 illustrates the preparation of Amphotericin B/Gelucire/Peceol/TPGS/Powdered excipients formulations.

FIG. 2 shows thermogravimetric analysis (TGA) curves of Amphotericin B (23%) for Formulations 1-3.

FIG. 3 shows the dissolution profiles of Amphotericin B for Formulations 1-3.

FIG. 4 shows the dissolution profiles of Amphotericin B in scale-up Formulation 1A and Formulation 1B at T=0/Initial compared to Formulation 1, Formulation 2.

FIG. 5 shows the dissolution profile in 0.5% SDS in water of solid and semi-solid Amphotericin B formulations in capsules.

FIG. 6 shows the dissolution profile in FeSSIF pH 5.8 of solid and semi-solid Amphotericin B formulations in capsules.

FIG. 7 shows the dissolution profiles of 100 mg capsules comprising lipid based formulations.

FIG. 8 shows the dissolution profile of Amphotericin B granular formulations in capsules at T=0 and under stability storage conditions.

FIG. 9 shows the dissolution profile of Amphotericin B lipid based capsules of Formula 5A at T=0 and under stability storage conditions.

FIG. 10A shows the blood plasma concentration curve in humans measured after 100 mg of amphotericin B.

FIG. 10B shows the blood plasma concentration curve in humans measured after 200 mg of amphotericin B.

FIG. 10C shows the blood plasma concentration curve in humans measured after 400 mg of amphotericin B.

FIG. 10D shows the blood plasma concentration curve in humans measured after 800 mg of amphotericin B.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

The term “pharmaceutically acceptable” means biologically or pharmacologically compatible for in-vivo use in animals or humans, and can mean approved by a regulatory agency of the Federal or a state government or listed in the U.S. Pharmacopeia or other generally recognized pharmacopeia for use in animals, and more particularly in humans.

The term “subject,” as used herein, comprises any and all organisms and includes the term “patient.” “Subject” may refer to a human or any other animal.

The term “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

As used herein, the term “about,” when located before a dosage amount or dosage range of a specific ingredient, refers to an amount or range above and/or below the stated amount or range that does not manifestly alter the therapeutic effect of the specific ingredient from the stated amount or range.

In various embodiments the solid dosage forms described herein comprise amphotericin B and a lipophilic component. In further embodiments, the amphotericin B solid dosage forms of the present disclosure can further include a second therapeutic agent, for example any of those disclosed herein.

The bioavailability of amphotericin B in the solid dosage forms described herein are at least equivalent (and, in some embodiments, superior) to conventional liquid formulations, such as those disclosed in U.S. Pat. No. 8,592,382 and 8,673,866, each of which are herein incorporated by reference in its entirety for all purposes. For example, the liquid formulation disclosed in U.S. Pat. No. 8,673,866 utilizes an isotropic mixture of lipophilic components (oils, surfactants, solvents, and co-solvents/surfactants) at a weight ratio relative to amphotericin B exceeding about 189 to 1 to achieve suitable levels of bioavailability. However, such levels of lipophilic components produces an oily formulation that causes gastric upset. The present inventors surprisingly and unexpectedly discovered that equivalent (and even superior) levels of bioavailability can be achieved with solid dosage forms comprising a significantly reduced amount of the lipophilic components, without causing gastric upset.

Importantly, the present inventors also surprisingly discovered that the present solid dosage forms significantly increase the subject's exposure to amphoteric B (e.g., in terms of the area under the blood plasma concentration curve (AUC) for amphotericin B) compared to conventional liquid dosage forms, such as MAT2203, an encochleated formulation of amphotericin B. Such properties of the presently disclosed solid dosage forms indicate efficacious treatment of the disease disclosed herein.

In some embodiments, the solid dosage forms of the present disclosure provide equivalent bioavailability to the above-referenced conventional liquid formulations, with a large ratio of amphotericin B relative to one or more lipophilic components of the formulation, whereas conventional liquid amphotericin formulations employ a large ratio of lipophilic components to amphotericin B in order to provide sufficient bioavailability. In embodiments, the solid compositions of the present disclosure have a weight ratio of amphotericin B to the lipophilic components in the range of about 100:1 to about 1:1, for example about 100:1, about 95:1, about 90:1, about 85:1, about 80:1, about 75:1, about 70:1, about 65:1, about 60:1, about 55: about 50:1, about 45:1 about 40:1, about 35:1, about 30:1, about 25:1, about 20:1, about 15:1, about 10:1, about 9.5:1 about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2.5:1, about 2:1, about 1.5:1, or about 1:1, inclusive of all ranges and subranges therebetween.

In other embodiments, the solid dosage forms of the present disclosure provide equivalent bioavailability to the above-referenced liquid formulations and have a smaller excess of the lipophilic components relative to amphotericin B compared to the conventional liquid formulations. In embodiments, the weight ratio of the one or more lipophilic components (e.g., one, two, three, etc., lipophilic components) in the solid dosage forms of the present disclosure to the amphotericin B is in the range of about 100:1 to about 1:1, for example about 100:1, about 95:1, about 90:1, about 85:1, about 80:1, about 75:1, about 70:1, about 65:1, about 60:1, about 55:1 about 50:1, about 45:1 about 40:1, about 35:1, about 30:1, about 25:1, about 20:1, about 15:1, about 10:1, about 9.5:1 about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2.5:1, about 2:1, about 1.5:1, or about 1:1, inclusive of all ranges and subranges therebetween.

In alternative embodiments, the solid dosage forms of the present disclosure comprise about 10-30 weight % amphotericin B and about 1-10 weight % (total) of the one or more lipophilic components. For example, the weight % amphotericin B is about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%, inclusive of all ranges and subranges therebetween; and the total weight % lipophilic components is about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10%, inclusive of all ranges and subranges therebetween.

In some embodiments, at least one lipophilic component is used in combination with the therapeutic agent (e.g. amphotericin B). In other embodiments, at least one lipophilic component is used to facilitate coating the therapeutic agent onto a solid carrier. The lipophilic component may include any hydrophobic material in which the therapeutic agent (e.g. amphotericin B) can be dissolved or suspended, and which is pharmaceutically acceptable. Lipophilic components used to solubilize the therapeutic agent may be selected based on the hydrophilic-lipophilic balance (HLB) of the therapeutic agent and the lipophilic component, or of the lipid and an optional organic solvent to facilitate solubilization of the amphotericin B in the lipophilic component. Suitable lipid materials for solubilizing the therapeutic agent (e.g. amphotericin B) may have an HLB value which is equal to that of the therapeutic agent or otherwise sufficient to solubilize the therapeutic agent in an appropriate solvent. For example, lipophilic components suitable to solubilize amphotericin B in ethanol may have an HLB of 14 or less (e.g., 13, 12, 11, or 10).

Each lipophilic component in the compositions of the present disclosure can be selected from natural (human-, animal-, or plant-derived) or synthetic sources. The lipophilic component can be a liquid or a solid at room temperature, provided that the solid can be melted upon heating and the melted lipophilic component does not degrade or denature the therapeutic agent (e.g. amphotericin B). In some embodiments, at least one lipophilic component may be used to solubilize the therapeutic agent (e.g. a lipophilic drug, e.g. amphotericin B). In other embodiments, the lipophilic component may be selected to improve the oral absorption of the therapeutic agent (amphotericin B). In further embodiments, the lipophilic component may be selected to improve the bioavailability of the therapeutic agent (e.g. amphotericin B). In still other embodiments, the lipophilic component may include a surfactant. In some such embodiments, the lipophilic component may be a non-ionic surfactant. In even further embodiments, the lipophilic component is a lipophilic binder material which promotes coating or adhesion of the therapeutic agent to a solid carrier.

In embodiments, the dosage forms disclosed herein may include one lipophilic component or a mixture of two or more lipophilic components (e.g., a mixture of 3 lipophilic components, 4 lipophilic components, 5 lipophilic components, etc.). In embodiments which entail two lipophilic components, the weight ratio of the first lipophilic component to the second lipophilic component is in the range of about 99:1 to about 1:99, for example about 99:1, about 95:5, about 90:10, about 85:15, about 80:20, about 75:25, about 70:30, about 65:35, about 60:40, about 55:45 about 50:50, about 45:55 about 40:60, about 35:65, about 30:70, about 25:75, about 20:80, about 15:85, about 10:90, about 5:95 and about 1:99, inclusive of all ranges and subranges therebetween.

Non-limiting examples of lipophilic components which are useful in the solid dosage forms disclosed herein include pharmaceutically acceptable fats, fatty substances, oils, phospholipids, sterols, and waxes. Fats generally refer to esters of glycerol (e.g., mono-, di- or triesters of glycerol and fatty acids). Suitable fats and fatty substances include but not limited to fatty alcohols (such as lauryl, myristyl, stearyl, cetyl or cetostearyl alcohol, etc.), fatty acids and derivatives, including but not limited to fatty acid esters, fatty acid glycerides (mono-, di- and tri-glycerides), and hydrogenated fats. Fats may be either solid or liquid at normal room temperature, depending on their structure and composition.

Suitable oils include pharmaceutically acceptable animal (e.g., fatty acid esters), mineral (e.g., paraffin oils), vegetable (e.g., vegetable oils), or synthetic hydrocarbons that are liquid at room temperature. Examples of pharmaceutically acceptable oils include but are not limited to: mineral oils such as paraffin oils; vegetable oils such as castor oils, hydrogenated vegetable oil, sesame oils, and peanut oils; and animal oils and fats such as triglycerides and butters. Partially hydrogenated vegetable oils are derived from natural products and generally comprise a mixture of glycerides of C₁₄₋₂₀ fatty acids, in particular palmitic and stearic acids. Suitable examples of partially hydrogenated vegetable oils include partially hydrogenated cottonseed oil, soybean oil, corn oil, peanut oil, palm oil, sunflower seed oil or mixtures thereof. Chemical equivalents of partially hydrogenated vegetable oils include synthetically produced glycerides of C₁₄₋₂₀ fatty acids having the same properties as the naturally derived products as hereinbefore described.

Suitable phospholipids include pharmaceutically acceptable plant, animal, and synthetic phospholipids. Examples of pharmaceutically acceptable phospholipids include cholines phosphatidylethanolamine, and phosphatidylglycerols, such as, but not limited to, phosphatidylcholine, 1,2-dierucoylphosphatidylcholine, 1,2-dimyristoylphosphatidylcholine, 1,2-dioleoylphosphatidylcholine, 1,2-dioleoylphosphatidylserine, 1,2-distearoylphosphatidylglycerol, 1,2-dipalmitoylphosphatidylcholine, 1,2-distearoylphosphatidylcholine, 1,2-distearoylphosphatidylglycerol, egg phosphatidylcholine, egg phosphatidylglycerol, soy phosphatidylcholine, glycerophosphocholine, hydrogenated soybean phosphatidylcholine, lysophosphatidylcholine, lysophosphatidylethanolamine, N-(carbonyl-methoxypoly ethylene glycol 2000)-1,2-distearoylphosphatidylethanol amine sodium salt, muramyltripeptide-phosphatidylethanolamine, 1-palmitoyl-2-linoleoylphosphatidylcholine, 1-palmitoyl-2-linoleoylphosphatidylglycerol, 1-palmitoyl-2-oleoylphosphatidylcholine, 1-palmitoyl-2-oleoylphosphatidylglycerol, polyenylphosphatidylcholine, 1-palmitoyl-2-stearoylphosphatidylcholine, 1-palmitoyl-2-stearoylphosphatidylglycerol, 1-stearoyl-2-linoleoylphosphatidylcholine, 1-stearoyl-2-linoleoylphosphatidylglycerol, sphingomyelin, 1-stearoyl-2-oleoyl phosphatidylcholine, 1-stearoyl-2-oleoyl phosphatidylglycerol, and the like.

Suitable waxes include animal waxes, plant waxes, mineral waxes, and petroleum waxes. Examples of waxes include, but are not limited to, glyceryl behenate, glyceryl monostearate, stearic acid, palmitic acid, lauric acid, carnauba wax, cetyl alcohol, glyceryl stearate beeswax, paraffin wax, ozokerite, candelilla wax, cetyl alcohol, stearyl alcohol, spermaceti, carnauba wax, bayberry wax, montan, ceresin, and microcrystalline waxes.

In particular embodiments, lipophilic components suitable for use in the solid dosage forms disclosed herein include fatty acid glycerol esters, polyethylene oxide-containing fatty acid esters, and combinations thereof.

In specific embodiments, the amphotericin B formulations of the present disclosure include one or more fatty acid glycerol esters. As used herein the term “fatty acid glycerol esters” refers to esters formed between glycerol and one or more fatty acids including mono-, di-, and tri-esters (i.e., glycerides). Suitable fatty acids include saturated and unsaturated fatty acids having from eight (8) to twenty-two (22) carbons atoms (i.e., C8-C22 fatty acids). In certain embodiments, suitable fatty acids include C12-C18 fatty acids. The fatty acid glycerol esters useful in the formulations can be provided by commercially available sources. A representative source for the fatty acid glycerol esters is a mixture of mono-, di-, and triesters commercially available as PECEOL® (Gattefosse, Saint Priest Cedex, France), commonly referred to as “glyceryl oleate” or “glyceryl monooleate.” In some embodiments, when PECEOL® is used as the source of fatty acid glycerol esters in the formulations, the fatty acid glycerol esters comprise from about 32 to about 52% by weight fatty acid monoglycerides, from about 30 to about 50% by weight fatty acid diglycerides, and from about 5 to about 20% by weight fatty acid triglycerides. The fatty acid glycerol esters comprise greater than about 60% by weight oleic acid (C18: 1) mono-, di-, and triglycerides. Other fatty acid glycerol esters include esters of palmitic acid (C16) (less than about 12%), stearic acid (C18) (less than about 6%), linoleic acid (C18:2) (less than about 35%), linolenic acid (C18:3) (less than about 2%), arachidic acid (C20) (less than about 2%), and eicosanoic acid (C20:1) (less than about 2%). PECEOL® can also include free glycerol (typically about 1%). In one embodiment, the fatty acid glycerol esters comprise about 44% by weight fatty acid monoglycerides, about 45% by weight fatty acid diglycerides, and about 9% by weight fatty acid triglycerides, and the fatty acid glycerol esters comprise about 75% by weight oleic acid (C18:1) mono-, di-, and triglycerides. Other fatty acid glycerol esters include esters of palmitic acid (C16) (about 4%), stearic acid (CI5) (about 2%), linoleic acid (CIS:2) (about 12%), linolenic acid (C18:3) (less than 1%), arachidic acid (C20) (less than 1%), and eicosanoic acid (C20:1) (less than 1%).

In embodiments, a fatty acid glycerol ester may be the sole lipid in the amphotericin B formulation. In other embodiments, the formulation may include a mixture fatty acid glycerol ester, for example any of those disclosed herein. In still other embodiments, one or more fatty acid glycerol ester may be used in combination with other lipophilic components as described herein, such one or more polyethylene oxide-containing fatty acid esters as described herein.

In some embodiments, the amphotericin B formulations described herein comprise at least one polyethylene oxide-containing lipophilic components, such as fatty acid esters. As used herein, the term “polyethylene oxide-containing fatty acid ester” refers to a fatty acid ester that includes a polyethylene oxide group (i.e., polyethylene glycol group) covalently coupled to the fatty acid through an ester bond. Polyethylene oxide-containing fatty acid esters include mono- and di-fatty acid esters of polyethylene glycol. Suitable polyethylene oxide-containing fatty acid esters are derived from fatty acids including saturated and unsaturated fatty acids having from eight (8) to twenty-two (22) carbons atoms (i.e., a polyethylene oxide ester of a C8-C22 fatty acid). In certain embodiments, suitable polyethylene oxide-containing fatty acid esters are derived from fatty acids including saturated and unsaturated fatty acids having from twelve (12) to eighteen (18) carbons atoms (i.e., a polyethylene oxide ester of a C12-C18 fatty acid). Representative polyethylene oxide-containing fatty acid esters include saturated C8-C22 fatty acid esters. In certain embodiments, suitable polyethylene oxide-containing fatty acid esters include saturated CI2-C18 fatty acids.

The molecular weight of the polyethylene oxide group of the polyethylene oxide-containing fatty acid ester can be varied to optimize the solubility of the therapeutic agent (e.g., amphotericin B) in the formulation. Representative average molecular weights for the polyethylene oxide groups can be from about 350 to about 2000. In one embodiment, the average molecular weight for the polyethylene oxide group is about 1500.

In some embodiments, when the amphotericin B formulation includes a polyethylene oxide-containing fatty acid in the lipophilic component, the lipophilic component may include only one type of polyethylene oxide-containing fatty acid. In other embodiments, the polyethylene oxide-containing fatty acid in the lipophilic component may include a mixture of polyethylene oxide-containing fatty acid esters (mono- and di-fatty acid esters of polyethylene glycol).

The polyethylene oxide-containing fatty acid esters useful in the formulations of the present disclosure can be provided by commercially available sources. Representative polyethylene oxide-containing fatty acid esters (mixtures of mono- and diesters) are commercially available under the designation GELUCIRE® (Gattefosse, Saint Priest Cedex, France). Suitable polyethylene oxide-containing fatty acid esters include GELUCIRE® 44/14, GELUCIRE® 50/13, GELUCIRE® 53/10, and GELUCIRE® 48/16. The numerals in these designations refer to the melting point and hydrophilic/lipophilic balance (HLB) of these materials, respectively. GELUCIRE® 44/14, GELUCIRE ® 50/13, GELUCIRE® 53/10 , and GELUCIRE® 48/16 are mixtures of (a) mono-, di-, and triesters of glycerol (glycerides) and (b) mono- and diesters of polyethylene glycol (macrogols). The GELUCIRES can also include free polyethylene glycol (e.g., PEG 1500).

Lauric acid (C12) is the predominant fatty acid component of the glycerides and polyethylene glycol esters in GELUCIRE® 44/14. GELUCIRE® 44/14 is referred to as a mixture of glyceryl dilaurate (lauric acid diester with glycerol) and PEG dilaurate (lauric acid diester with polyethylene glycol), and is commonly known as PEG-32 glyceryllaurate (Gattefosse) lauroyl macrogol-32 glycerides EP, or lauroyl polyoxylglycerides USP/NF. GELUCIRE® 44/14 is produced by the reaction of hydrogenated palm kernel oil with polyethylene glycol (average molecular weight 1500). GELUCIRE® 44/14 includes about 20% mono-, di- and, triglycerides, about 72% mono- and di-fatty acid esters of polyethylene glycol 1500, and about 8% polyethylene glycol 1500.

GELUCIRE® 44/14 includes lauric acid (C12) esters (30 to 50%), myristic acid (C14) esters (5 to 25%), palmitic acid (C16) esters (4 to 25%), stearic acid (C18) esters (5 to 35%), caprylic acid (C8) esters (less than 15%), and capric acid (C10) esters (less than 12%). GELUCIRE® 44/14 may also include free glycerol (typically less than about 1%). In a representative formulation, GELUCIRE® 44/14 includes lauric acid (C12) esters (about 47%), myristic acid (C14) esters (about 18%), palmitic acid (C16) esters (about 10%), stearic acid (C18) esters (about 11%), caprylic acid (C8) esters (about 8%), and capric acid (C10) esters (about 12%).

Palmitic acid (C16) (40-50%) and stearic acid (C18) (48-58%) are the predominant fatty acid components of the glycerides and polyethylene glycol esters in GELUCIRE® 50/13. GELUCIRE® 50/13 is known as PEG-32 glyceryl palmitostearate (Gattefosse), stearoyl macrogolglycerides EP, or stearoyl polyoxylglycerides USP/NF). GELUCIRE® 50/13 includes palmitic acid (C16) esters (40 to 50%), stearic acid (C18) esters (48 to 58%) (stearic and palmitic acid esters greater than about 90%), lauric acid (C12) esters (less than 5%), myristic acid (C14) esters (less than 5%), caprylic acid (C8) esters (less than 3%), and capric acid (C10) esters (less than 3%). GELUCIRE® 50/13 may also include free glycerol (typically less than about 1%). In a representative formulation, GELUCIRE® 50/13 includes palmitic acid (C16) esters (about 43%), stearic acid (CIS) esters (about 54%) (stearic and palmitic acid esters about 97%), lauric acid (C12) esters (less than 1%), myristic acid (C14) esters (about 1%), caprylic acid (C8) esters (less than 1%), and capric acid (C10) esters (less than 1%) Stearic acid (C18) is the predominant fatty acid component of the glycerides and polyethylene glycol esters in GELUCIRE ® 53/10. GELUCIRE® 53/10 is known as PEG-32 glyceryl stearate (Gattefosse).

In one embodiment, the polyethylene oxide-containing fatty acid ester is a lauric acid ester, a palmitic acid ester, or a stearic acid ester (i.e., mono- and di-lauric acid esters of polyethylene glycol, mono- and di-palmitic acid esters of polyethylene glycol, mono- and di-stearic acid esters of polyethylene glycol). Mixtures of these esters can also be used.

In some embodiments, the solid dosage form comprises at least one fatty acid glycerol ester and at least one polyethylene oxide-containing fatty acid ester. In such embodiments, the ratio of the at least one fatty acid glycerol ester to the at least one polyethylene oxide-containing fatty acid ester is in the range of from about 90:10 to about 10:90, including about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, or about 10:90, inclusive of all ranges and subranges therebetween. In further embodiments, the solid dosage form comprises PECEOL® and GELUCIRE® 44/14 (as described herein). In embodiments, the ratio of PECEOL® and GELUCIRE® 44/14 is in the range of from about 90:10 to about 10:90, including about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, or about 10:90, inclusive of all ranges and subranges therebetween.

The amphotericin B formulations disclosed herein optionally include a stabilizer. In some embodiments, the stabilizer is a thermal stabilizer, for example tocopherol polyethylene glycol succinate (e.g., TPGS or vitamin E TPGS). In some embodiments, the stabilizer is an antioxidant, such as butylated hydroxyanisole (BHA) or butylated hydroxytoluene (BHT). Such thermal stabilizers and/or antioxidants enhance the thermal stability of the formulation, which in turn, can increase the formulation's shelf-life, which is particularly important in tropical regions of the world where prolonged exposure to high temperatures are common and refrigerated medicinal storage is difficult.

Structurally, tocopherol polyethylene glycol succinates have a polyethylene glycol (PEG) covalently coupled to tocopherol (e.g., a-tocopherol or vitamin E) through a succinate linker. Because PEG is a polymer, a variety of polymer molecular weights can be used to prepare the TPGS. In one embodiment, the TPGS is tocopherol polyethylene glycol succinate 1000, in which the average molecular weight of the PEG is 1000. One suitable tocopherol polyethylene glycol succinate is vitamin E TPGS commercially available from Eastman.

In some embodiments, the solid dosage forms of the present disclosure comprise a dosage of amphotericin B in the range of from about 1 mg to about 500 mg, including about 1 mg, about 5 mg, about 10 mg, 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 g, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg, inclusive of all ranges and subranges therebetween.

In some embodiments, the % w/w of amphotericin B in the solid dosage form is at least about 1%, or at least about 5%, or about at least about 10%, or at least about 15%, or about 20%, or at least about 25%, or at least about 30%, or at least about 35%, or at least about 40%, or at least about 45%, or at least about 50%, or at least about 55%, or at least about 60%, or at least about 65%, or at least about 70%. In some embodiments, the % w/w of amphotericin B in the solid dosage form is in the range of about 1% to about 70%, or about 5% to about 70%, or about 5% to about 60%, or about 5% to about 50%, or about 5% to about 40%, or about 10% to about 40%, or about 15% to about 40%, or about 20% to about 40%, or about 20% to about 40%, or about 20% to about 35%, or about 20% to about 30%.

The solid dosage forms of the present disclosure can be prepared by any suitable method, including granulation of the therapeutic agent (e.g. amphotericin B) with excipients (e.g. fillers, glidants, lubricants, etc. known in the art and described herein), extrusion of the therapeutic agent with excipients, direct compression of the therapeutic agent with excipients to form tablets, etc.

In particular embodiments, the solid dosage forms the present disclosure can be prepared by coating the active agent, e.g. amphotericin B on a solid carrier. The solid carrier can be any material upon which a drug-containing composition can be coated and which is suitable for human consumption. Any conventional coating process can be used. For example, the therapeutic agent, e.g. amphotericin B can be dissolved or suspended in a suitable solvent (e.g., ethanol), together with an optional binder, or alternatively one or more of the lipophilic components described herein, and deposited on the solid carrier by methods known in the art, e.g. fluidized bed coating or pan coating methods. The solvent can be removed e.g. by drying, or in situ during the coating process (e.g., during fluidized bed coating), and/or in a subsequent drying step.

In some embodiments, the solid carrier may be an inert bead or an inert particle. In other embodiments, the solid carrier a non-pareil seed, an acidic buffer crystal, an alkaline buffer crystal, or an encapsulated buffer crystal.

In some embodiments, the solid carrier may be a sugar sphere, cellulose sphere, lactose sphere, lactose-microcrystalline cellulose (MCC) sphere, mannitol-MCC sphere, or silicon dioxide sphere.

In other embodiments, the solid carrier may be a saccharide, a sugar alcohol, or combinations thereof. Suitable saccharides include lactose, sucrose, maltose, and combinations thereof. Suitable sugar alcohols include mannitol, sorbitol, xylitol, maltitol, arabitol, ribitol, dulcitol, iditol, isomalt, lactitol, erythritol and combinations thereof.

In embodiments, the solid carrier may be formed by combining any of the above with a filler. Examples of suitable fillers which may be used to form a solid carrier include lactose, microcrystalline cellulose, silicified microcrystalline cellulose, mannitol-microcrystalline cellulose and silicon dioxide.

In other embodiments, the dosage form disclosed herein does not include a solid carrier.

In embodiments, the solid dosage forms disclosed herein can include one or more pharmaceutically acceptable excipients. Pharmaceutically acceptable excipients include fillers, diluents, glidants, disintegrants, binders and lubricants. Other pharmaceutically acceptable excipients include acidifying agents, alkalizing agents, preservatives, antioxidants, buffering agents, chelating agents, coloring agents, complexing agents, emulsifying and/or solubilizing agents, flavors, perfumes, humectants, sweetening agents and wetting agents.

Examples of suitable fillers and/or binders include lactose (e.g. spray-dried lactose, a-lactose, P-lactose, Tabletose®, various grades of Pharmatose®, Microtose® or FastFlo®), microcrystalline cellulose (various grades of Avicel®, Ceolus®, Elcema®, Vivacel®, Ming Tai® or Solka-Floc®), hydroxypropylcellulose, L-hydroxypropylcellulose (low substituted), low molecular weight hydroxypropyl methylcellulose (HPMC) (e.g. Methocel E, F and K from Dow Chemical, Metolose SH from Shin-Etsu, Ltd), hydroxyethylcellulose, sodium carboxymethylcellulose, carboxymethylhydroxyethylcellulose and other cellulose derivatives, sucrose, agarose, sorbitol, mannitol, xylitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, polyvinylpyrrolidone, and polyethylene glycol.

Examples of pharmaceutically acceptable diluents include calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose and sugar.

Pharmaceutically acceptable disintegrants include alginic acid, carboxymethylcellulose calcium, carboxymethylcellulose sodium (e.g., AC-DI-SOL® and Primellose®), colloidal silicon dioxide, croscarmellose sodium, crospovidone (e.g., Kollidon® and Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), potato starch, and starch.

Examples of pharmaceutically acceptable glidants include colloidal silicon dioxide, magnesium trisilicate, powdered cellulose, starch, talc, and tribasic calcium phosphate.

Pharmaceutically acceptable lubricants include stearic acid, magnesium stearate, calcium stearate or other metallic stearates (e.g., zinc stearate), glyceryl monostearate, glyceryl palmitostearate, waxes and glycerides, hydrogenated castor oil, hydrogenated vegetable oil, light mineral oil, polyethylene glycol, glyceryl behenate, colloidal silica, hydrogenated vegetable oils, com starch, sodium lauryl sulfate, sodium stearyl fumarate, polyethylene glycols, alkyl sulfates, sodium benzoate, talc, and sodium acetate.

Flavoring agents and flavor enhancers make the dosage form more palatable to the patient. Common flavoring agents and flavor enhancers for pharmaceutical products that may be included in the composition and/or combination of the present invention include maltol, vanillin, ethyl vanillin, menthol, citric acid, fumaric acid, ethyl maltol, and tartaric acid.

Solid compositions may also be dyed using any pharmaceutically acceptable colorant to improve their appearance and/or facilitate patient identification of the product and unit dosage level.

The compositions disclosed herein can be formulated as a solid dosage form. Suitable solid dosage forms include tablets and capsules, such as a gelatin capsule or suitable synthetic capsules known in the art, such as HPMC (hydroxypropyl methylcellulose) capsules.

In embodiments, the solid dosage form described herein may be made by:
(a) dissolving at least one lipid and the therapeutic agent in a solvent, thereby forming a liquid mixture comprising the therapeutic agent;
(b) coating the mixture comprising a therapeutic agent on a solid carrier; and
(c) removing the solvent; thereby forming drug coated-particles.

Any solvent in which the lipophilic component and the therapeutic agent can be dissolved can be used to make the solid dosage forms described herein. Examples of suitable solvents include lipophilic solvents, such as lipophilic organic solvents. Non-limiting examples of solvents include alcohols (e.g., ethanol, propanol, isopropanol, and the like), ketones (e.g., acetone and the like), dimethyl sulfoxide, dichloromethane, and the like.

The drug-coated particles can be milled as needed and passed through one or more mesh screens to produce granules having a desired size range. In various embodiments, the drug-coated particles may have an average particle size ranging from 10-2000 μm, e.g., 100-1000 μm, or 500-1000 μm.

In embodiments, the drug-coated particles can be filled into a capsule or compressed, optionally in combination with various excipients as described herein into a tablet.

In other embodiments, the therapeutic agent, e.g. amphotericin B, and an appropriate amount of a melt of room temperature solid lipophilic components (as described herein) can be mixed together (for example using methods, but not compositions, disclosed in U.S. Pat. No. 8,592,382 and 8,673,866), optionally with a suitable amount of a solvent such as ethanol, until a homogeneous mixture or solution is formed. The resulting mixture or solution is then allowed to cool to thereby form a semi-solid composition. The semi-solid composition can then filled into a gelatin capsule to thereby provide a solid-dosage form.

In embodiments, the amphotericin B dosage forms disclosed herein are bioequivalent to conventional liquid formulations. That is, the solid dosage forms have an average maximum blood plasma concentration (Cₘₐₓ), an average area under the blood plasma concentration curve (AUC), and an average time to reach Cₘₐₓ (Tₘₐₓ) which is within the about 80% to about 125% of each of the average Cₘₐₓ, average AUC, and average Tₘₐₓ of conventional liquid compositions when administered to a human. In some embodiments, the solid dosage forms have a Cₘₐₓ, and Tₘₐₓ that is similar (e.g. bioequivalent) to liquid or e Cₘₐₓ, AUC, and Tₘₐₓ, as used herein, refer to the averages of such values measured for a population of subjects.

In some embodiments, the solid dosage forms described herein provide an average Cₘₐₓ within about 80%-125% of the range of from about 21.09 ng/mL to about 42.07 ng/mL, after a single dose of about 100-800 mg of AmpB (inclusive of all values and subranges therebetween), e.g., about 14 ng/mL, about 14.5 ng/mL, about 15 ng/mL, about 15.5 ng/mL, about 16 ng/mL, about 16.5 ng/mL, about 17 ng/mL, about 17.5 ng/mL, about 18 ng/mL, about 18.5 ng/mL, about 19 ng/mL, about 19.5 ng/mL, about 20 ng/mL, about 20.5 ng/mL, about 21 ng/mL, about 21.5 ng/mL, about 22 ng/mL, about 22.5 ng/mL, about 23 ng/mL, about 23.5 ng/mL, about 24 ng/mL, about 24.5 ng/mL, about 25 ng/mL, about 25.5 ng/mL, about 30 ng/mL, about 30.5 ng/mL, about 31 ng/mL, about 31.5 ng/mL, about 32 ng/mL, about 32.5 ng/mL, about 33 ng/mL, about 33.5 ng/mL, about 34 ng/mL, about 34.5 ng/mL, about 35 ng/mL, about 35.5 ng/mL, about 36 ng/mL, about 36.5 ng/mL, about 37 ng/mL, about 37.5 ng/mL, about 38 ng/mL, about 38.5 ng/mL, about 39 ng/mL, about 39.5 ng/mL, about 40 ng/mL, about 40.5 ng/mL, about 41 ng/mL, about 41.5 ng/mL, about 42 ng/mL, about 42.5 ng/mL, about 43 ng/mL, about 43.5 ng/mL, about 44 ng/mL, about 44.5 ng/mL, about 45 ng/mL, about 45.5 ng/mL, about 46 ng/mL, about 46.5 ng/mL, about 47 ng/mL, about 47.5 ng/mL, about 48 ng/mL, about 48.5 ng/mL, about 49 ng/mL, about 49.5 ng/mL, about 50 ng/mL, about 50.5 ng/mL, about 51 ng/mL, about 51.5 ng/mL, about 52 ng/mL, about 52.5 ng/mL, about 53 ng/mL, about 53.5 ng/mL, about 54 ng/mL, about 54.5 ng/mL, about 55 ng/mL, about 55.5 ng/mL, about 56 ng/mL, about 56.5 ng/mL, about 57 ng/mL, about 57.5 ng/mL, about 58 ng/mL, about 58.5 ng/mL, about 59 ng/mL, about 59.5 ng/mL, and about 60 ng/mL inclusive of all values and subranges therebetween.

In some embodiments, the solid dosage forms described herein provide an average Tₘₐₓ within about 80%-125% of the range of from about 5.25 hr to about 9.66 hr after a single dose of about 100 to about 400 mg of AmpB (inclusive of all values and subranges therebetween) e.g., about 3.7 hr, about 3.8 hr, about 3.9 hr, about 4 hr, about 4.1 hr, about 4.2 hr, about 4.3 hr, about 4.4 hr, about 4.5 hr, about 4.6 hr, about 4.7 hr, about 4.8 hr, about 4.9 hr, about 5 hr, about 5.1 hr, about 5.2 hr, about 5.3 hr, about 5.4 hr, about 5.5 hr, about 5.6 hr, about 5.7 hr, about 5.8 hr, about 5.9 hr about 6 hr, about 6.1 hr, about 6.2 hr, about 6.3 hr, about 6.4 hr, about 6.5 hr, about 6.6 hr, about 6.7 hr, about 6.8 hr, about 6.9 hr, about 7 hr, about 7.1 hr, about 7.2 hr, about 7.3 hr, about 7.4 hr, about 7.5 hr, about 7.6 hr, about 7.7 hr, about 7.8 hr, about 7.9 hr, about 8 hr, about 8.1 hr, about 8.2 hr, about 8.3 hr, about 8.4 hr, about 8.5 hr, about 8.6 hr, about 8.7 hr, about 8.8 hr, about 8.9 hr, about 9 hr, about 9.1 hr, about 9.2 hr, about 9.3 hr, about 9.4 hr, about 9.5 hr, about 9.6 hr, about 9.7 hr, about 9.8 hr, about 9.9 hr, about 10 hr, about 10.1 hr, about 10.2 hr, about 10.3 hr, about 10.4 hr, about 10.5 hr, about 10.6 hr, about 10.7 hr, about 10.8 hr, about 10.9 hr, about 10 hr, about 10.1 hr, about 10.2 hr, about 10.3 hr, about 10.4 hr, about 10.5 hr, about 10.6 hr, about 10.7 hr, about 10.8 hr, about 10.9 hr, about 11 hr, about 11.1 hr, about 11.2 hr, about 11.3 hr, about 11.4 hr, about 11.5 hr, about 11.6 hr, about 11.7 hr, about 11.8 hr, about 11.9 hr, about 12 hr, about 12.1 hr, about 12.2 hr, about 12.3 hr, about 12.4 hr, about 12.5 hr, about 12.6 hr, about 12.7 hr, about 12.8 hr, about 12.9 hr, about 13 hr, about 13.1 hr, about 13.2 hr, about 13.3 hr, about 13.4 hr, and about 13.5 hr, inclusive of all values and subranges therebetween.

In some embodiments, the solid dosage forms described herein provide an average AUC₀₋ₜ within about 80%-125% of the range of from about 510.00 hr*ng/mL to about 4779.45 hr*ng/mL after a single dose of about 100-800 mg of AmpB (inclusive of all values and subranges therebetween), e.g., about 450 hr*ng/mL, about 475 hr*ng/mL, about 500 hr*ng/mL, about 525 hr*ng/mL, about 550 hr*ng/mL, about 575 hr*ng/mL, about 600 hr*ng/mL, about 625 hr*ng/mL, about 650 hr*ng/mL, about 675 hr*ng/mL, about 700 hr*ng/mL, about 725 hr*ng/mL, about 750 hr*ng/mL, about 775 hr*ng/mL, about 800 hr*ng/mL, about 825 hr*ng/mL, about 850 hr*ng/mL, about 875 hr*ng/mL, about 900 hr*ng/mL, about 925 hr*ng/mL, about 950 hr*ng/mL, about 975 hr*ng/mL, about 1000 hr*ng/mL, about 1025 hr*ng/mL, about 1050 hr*ng/mL, about 1075 hr*ng/mL, about 1100 hr*ng/mL, about 1125 hr*ng/mL, about 1150 hr*ng/mL, about 1175 hr*ng/mL, about 1200 hr*ng/mL, about 1225 hr*ng/mL, about 1250 hr*ng/mL, about 1275 hr*ng/mL, about 1300 hr*ng/mL, about 1325 hr*ng/mL, about 1350 hr*ng/mL, about 1375 hr*ng/mL, about 1400 hr*ng/mL, about 1425 hr*ng/mL, about 1450 hr*ng/mL, about 1475 hr*ng/mL, about 1500 hr*ng/mL, about 1525 hr*ng/mL, about 1550 hr*ng/mL, about 1575 hr*ng/mL, about 1600 hr*ng/mL, about 1625 hr*ng/mL, about 1650 hr*ng/mL, about 1675 hr*ng/mL, about 1700 hr*ng/mL, about 1725 hr*ng/mL, about 1750 hr*ng/mL, about 1775 hr*ng/mL, about 1800 hr*ng/mL, about 1825 hr*ng/mL, about 1850 hr*ng/mL, about 1875 hr*ng/mL, about 1900 hr*ng/mL, about 1925 hr*ng/mL, about 1950 hr*ng/mL, about 1975 hr*ng/mL, about 2000 hr*ng/mL, about 2025 hr*ng/mL, about 2050 hr*ng/mL, about 2075 hr*ng/mL, about 2100 hr*ng/mL, about 2125 hr*ng/mL, about 2150 hr*ng/mL, and about 2175 hr*ng/mL, inclusive of all values and subranges therebetween.

In some embodiments, the solid dosage forms described herein compositions provide an average AUC_{0-inf} within about 80%-125% of the range of from about 509.84 hr*ng/mL to about 18366.24 hr*ng/mL after a single dose of about 100-800 mg of AmpB (inclusive of all values and subranges therebetween), e.g., about 400 hr*ng/mL, about 500 hr*ng/mL, about 600 hr*ng/mL, about 700 hr*ng/mL, about 800 hr*ng/mL, about 900 hr*ng/mL, about 1000 hr*ng/mL, about 1100 hr*ng/mL, about 1200 hr*ng/mL, about 1300 hr*ng/mL, about 1400 hr*ng/mL, about 1500 hr*ng/mL, about 1600 hr*ng/mL, about 1700 hr*ng/mL, about 1800 hr*ng/mL, about 1900 hr*ng/mL, about 2000 hr*ng/mL, about 2100 hr*ng/mL, about 2200 hr*ng/mL, about 2300 hr*ng/mL, about 2400 hr*ng/mL, about 2500 hr*ng/mL, about 2600 hr*ng/mL, about 2700 hr*ng/mL, about 2800 hr*ng/mL about 2900 hr*ng/mL, about 3000 hr*ng/mL, about 3100 hr*ng/mL, about 3200 hr*ng/mL, about 3300 hr*ng/mL, about 3400 hr*ng/mL, about 3500 hr*ng/mL, about 3600 hr*ng/mL, about 3700 hr*ng/mL, about 3800 hr*ng/mL, about 3900 hr*ng/mL, about 4000 hr*ng/mL, about 4100 hr*ng/mL, about 4200 hr*ng/mL, about 4300 hr*ng/mL, about 4400 hr*ng/mL, about 4500 hr*ng/mL, about 4600 hr*ng/mL, about 4700 hr*ng/mL, about 4800 hr*ng/mL, about 4900 hr*ng/mL, about 5000 hr*ng/mL, about 5100 hr*ng/mL, about 5200 hr*ng/mL, about 5300 hr*ng/mL, about 5400 hr*ng/mL, about 3500 hr*ng/mL, about 5600 hr*ng/mL, about 5700 hr*ng/mL, about 5800 hr*ng/mL, about 5900 hr*ng/mL, about 6000 hr*ng/mL, about 6100 hr*ng/mL, about 6200 hr*ng/mL, about 6300 hr*ng/mL, about 6400 hr*ng/mL, about 6500 hr*ng/mL, about 6600 hr*ng/mL, about 6700 hr*ng/mL, about 6800 hr*ng/mL, about 6900 hr*ng/mL, about 7000 hr*ng/mL, about 7100 hr*ng/mL, about 7200 hr*ng/mL, about 7300 hr*ng/mL, about 7400 hr*ng/mL, about 7500 hr*ng/mL, about 7600 hr*ng/mL, about 7700 hr*ng/mL, about 7800 hr*ng/mL, about 7900 hr*ng/mL, about 8000 hr*ng/mL, about 8100 hr*ng/mL, about 8200 hr*ng/mL, about 8300 hr*ng/mL, about 8400 hr*ng/mL, about 8500 hr*ng/mL, about 8600 hr*ng/mL, about 8700 hr*ng/mL, about 8800 hr*ng/mL, about 8900 hr*ng/mL, about 9000 hr*ng/mL, about 9100 hr*ng/mL, about 9200 hr*ng/mL, about 9300 hr*ng/mL, about 9400 hr*ng/mL, about 9500 hr*ng/mL, about 9600 hr*ng/mL, about 9700 hr*ng/mL, about 9800 hr*ng/mL, about 9900 hr*ng/mL, about 10000 hr*ng/mL, about 10100 hr*ng/mL, about 10200 hr*ng/mL, about 10300 hr*ng/mL, about 10400 hr*ng/mL, about 10500 hr*ng/mL, about 10600 hr*ng/mL, about 10700 hr*ng/mL, about 10800 hr*ng/mL, about 10900 hr*ng/mL, about 11000 hr*ng/mL, about 11100 hr*ng/mL, about 11200 hr*ng/mL, about 11300 hr*ng/mL, about 11400 hr*ng/mL, about 11500 hr*ng/mL, about 11600 hr*ng/mL, about 11700 hr*ng/mL, about 11800 hr*ng/mL, about 11900 hr*ng/mL, about 12000 hr*ng/mL, about 12100 hr*ng/mL, about 12200 hr*ng/mL, about 12300 hr*ng/mL, about 12400 hr*ng/mL, about 12500 hr*ng/mL, about 12600 hr*ng/mL, about 12700 hr*ng/mL, about 12800 hr*ng/mL, about 12900 hr*ng/mL, about 13000 hr*ng/mL, about 13100 hr*ng/mL, about 13200 hr*ng/mL, about 13300 hr*ng/mL, about 13400 hr*ng/mL, about 13500 hr*ng/mL, about 13600 hr*ng/mL, about 13700 hr*ng/mL, about 13800 hr*ng/mL, about 13900 hr*ng/mL, about 14000 hr*ng/mL, about 15100 hr*ng/mL, about 15200 hr*ng/mL, about 15300 hr*ng/mL, about 15400 hr*ng/mL, about 15500 hr*ng/mL, about 15600 hr*ng/mL, about 15700 hr*ng/mL, about 15800 hr*ng/mL, about 15900 hr*ng/mL, about 16000 hr*ng/mL, about 16100 hr*ng/mL, about 16200 hr*ng/mL, about 16300 hr*ng/mL, about 16400 hr*ng/mL, about 16500 hr*ng/mL, about 16600 hr*ng/mL, about 16700 hr*ng/mL, about 16800 hr*ng/mL, about 16900 hr*ng/mL, about 17000 hr*ng/mL, about 17100 hr*ng/mL, about 17200 hr*ng/mL, about 17300 hr*ng/mL, about 17400 hr*ng/mL, about 17500 hr*ng/mL, about 17600 hr*ng/mL, about 17700 hr*ng/mL, about 17800 hr*ng/mL, about 17900 hr*ng/mL, about 18000 hr*ng/mL, about 18100 hr*ng/mL, about 18200 hr*ng/mL, about 18300 hr*ng/mL, about 18400 hr*ng/mL, about 18500 hr*ng/mL, about 18600 hr*ng/mL, about 18700 hr*ng/mL, about 18800 hr*ng/mL, about 18900 hr*ng/mL, about 19000 hr*ng/mL, about 19100 hr*ng/mL, about 19200 hr*ng/mL, about 19300 hr*ng/mL, about 19400 hr*ng/mL, about 19500 hr*ng/mL, about 19600 hr*ng/mL, about 19700 hr*ng/mL, about 19800 hr*ng/mL, about 19900 hr*ng/mL, about 20000 hr*ng/mL, inclusive of all values and subranges therebetween.

In some embodiments, the solid dosage forms described herein provide an average Cₘₐₓ within about 80%-125% of the range of about 30.22±7.84 ng/mL, after a single dose of 100 mg of AmpB, e.g., about 15.5 ng/mL, about 16 ng/mL, about 16.5 ng/mL, about 17 ng/mL, about 17.5 ng/mL, about 18 ng/mL, about 18.5 ng/mL, about 19 ng/mL, about 19.5 ng/mL, about 20 ng/mL, about 20.5 ng/mL, about 21 ng/mL, about 21.5 ng/mL, about 22 ng/mL, about 22.5 ng/mL, about 23 ng/mL, about 23.5 ng/mL, about 24 ng/mL, about 24.5 ng/mL, about 25 ng/mL, about 25.5 ng/mL, about 30 ng/mL, about 30.5 ng/mL, about 31 ng/mL, about 31.5 ng/mL, about 32 ng/mL, about 32.5 ng/mL, about 33 ng/mL, about 33.5 ng/mL, about 34 ng/mL, about 34.5 ng/mL, about 35 ng/mL, about 35.5 ng/mL, about 36 ng/mL, about 36.5 ng/mL, about 37 ng/mL, about 37.5 ng/mL, about 38 ng/mL, about 38.5 ng/mL, about 39 ng/mL, about 39.5 ng/mL, about 40 ng/mL, about 40.5 ng/mL, about 41 ng/mL, about 41.5 ng/mL, about 42 ng/mL, about 42.5 ng/mL, about 43 ng/mL, about 43.5 ng/mL, about 44 ng/mL, about 44.5 ng/mL, about 45 ng/mL, about 45.5 ng/mL, about 46 ng/mL, about 46.5 ng/mL, about 47 ng/mL, about 47.5 ng/mL, and about 48 ng/mL, inclusive of all values and subranges therebetween.

In some embodiments, the solid dosage forms described herein provide an average Tₘₐₓ within 80%-125% of the range of about 6 hr after a single dose of 100 mg of AmpB, e.g., about 4 hr, about 4.1 hr, about 4.2 hr, about 4.3 hr, about 4.4 hr, about 4.5 hr, about 4.6 hr, about 4.7 hr, about 4.8 hr, about 4.9 hr, about 5 hr, about 5.1 hr, about 5.2 hr, about 5.3 hr, about 5.4 hr, about 5.5 hr, about 5.6 hr, about 5.7 hr, about 5.8 hr, about 5.9 hr about 6 hr, about 6.1 hr, about 6.2 hr, about 6.3 hr, about 6.4 hr, about 6.5 hr, about 6.6 hr, about 6.7 hr, about 6.8 hr, about 6.9 hr, about 7 hr, about 7.1 hr, about 7.2 hr, about 7.3 hr, about 7.4 hr, about 7.5 hr, about 7.6 hr, about 7.7 hr, about 7.8 hr, about 7.9 hr, and about 8 hr, inclusive of all values and subranges therebetween.

In some embodiments, the solid dosage forms described herein provide an average AUCO-t within about 80%-125% of the range of about 1228.86±710.86 hr*ng/mL after a single dose of 100 mg of AmpB, e.g., about 300 hr*ng/mL, about 325 hr*ng/mL, about 350 hr*ng/mL, about 375 hr*ng/mL, about 400 hr*ng/mL, about 425 hr*ng/mL, about 450 hr*ng/mL, about 475 hr*ng/mL, about 500 hr*ng/mL, about 525 hr*ng/mL, about 550 hr*ng/mL, about 575 hr*ng/mL, about 600 hr*ng/mL, about 625 hr*ng/mL, about 650 hr*ng/mL, about 675 hr*ng/mL, about 700 hr*ng/mL, about 725 hr*ng/mL, about 750 hr*ng/mL, about 775 hr*ng/mL, about 800 hr*ng/mL, about 825 hr*ng/mL, about 850 hr*ng/mL, about 875 hr*ng/mL, about 900 hr*ng/mL, about 925 hr*ng/mL, about 950 hr*ng/mL, about 975 hr*ng/mL, about 1000 hr*ng/mL, about 1025 hr*ng/mL, about 1050 hr*ng/mL, about 1075 hr*ng/mL, about 1100 hr*ng/mL, about 1125 hr*ng/mL, about 1150 hr*ng/mL, about 1175 hr*ng/mL, about 1200 hr*ng/mL, about 1225 hr*ng/mL, about 1250 hr*ng/mL, about 1275 hr*ng/mL, about 1300 hr*ng/mL, about 1325 hr*ng/mL, about 1350 hr*ng/mL, about 1375 hr*ng/mL, about 1400 hr*ng/mL, about 1425 hr*ng/mL, about 1450 hr*ng/mL, about 1475 hr*ng/mL, about 1500 hr*ng/mL, about 1525 hr*ng/mL, about 1550 hr*ng/mL, about 1575 hr*ng/mL, about 1600 hr*ng/mL, about 1625 hr*ng/mL, about 1650 hr*ng/mL, about 1675 hr*ng/mL, about 1700 hr*ng/mL, about 1725 hr*ng/mL, about 1750, about 1775 hr*ng/mL, about 1800 hr*ng/mL, about 1825 hr*ng/mL, about 1850 hr*ng/mL, about 1875 hr*ng/mL, about 1900 hr*ng/mL, about 1925 hr*ng/mL, about 1950 hr*ng/mL, about 1975 hr*ng/mL, about 2000 hr*ng/mL, about 2025 hr*ng/mL, about 2050 hr*ng/mL, about 2075 hr*ng/mL, about 2100 hr*ng/mL, about 2125 hr*ng/mL, about 2150 hr*ng/mL, about 2175 hr*ng/mL, about 2200 hr*ng/mL, about 2225 hr*ng/mL, about 2250 hr*ng/mL, about 2275 hr*ng/mL, about 2300 hr*ng/mL, about 2325 hr*ng/mL, about 2350 hr*ng/mL, about 2375 hr*ng/mL, about 2400 hr*ng/mL, about 2425 hr*ng/mL, about 2450 hr*ng/mL, about 2475 hr*ng/mL, about 2500 hr*ng/mL, about 2525 hr*ng/mL, about 2550 hr*ng/mL, about 2575 hr*ng/mL, and about 2600 hr*ng/mL, inclusive of all values and subranges therebetween.

In some embodiments, the solid dosage forms described herein provide an average AUC0-inf within about 80%-125% of the range of about 1083.35±269.57 hr*ng/mL after a single dose of 100 mg of AmpB, e.g., about 300 hr*ng/mL, about 325 hr*ng/mL, about 350 hr*ng/mL, about 375 hr*ng/mL, about 400 hr*ng/mL, about 425 hr*ng/mL, about 450 hr*ng/mL, about 475 hr*ng/mL, about 500 hr*ng/mL, about 525 hr*ng/mL, about 550 hr*ng/mL, about 575 hr*ng/mL, about 600 hr*ng/mL, about 625 hr*ng/mL, about 650 hr*ng/mL, about 675 hr*ng/mL, about 700 hr*ng/mL, about 725 hr*ng/mL, about 750 hr*ng/mL, about 775 hr*ng/mL, about 800 hr*ng/mL, about 825 hr*ng/mL, about 850 hr*ng/mL, about 875 hr*ng/mL, about 900 hr*ng/mL, about 925 hr*ng/mL, about 950 hr*ng/mL, about 975 hr*ng/mL, about 1000 hr*ng/mL, about 1025 hr*ng/mL, about 1050 hr*ng/mL, about 1075 hr*ng/mL, about 1100 hr*ng/mL, about 1125 hr*ng/mL, about 1150 hr*ng/mL, about 1175 hr*ng/mL, about 1200 hr*ng/mL, about 1225 hr*ng/mL, about 1250 hr*ng/mL, about 1275 hr*ng/mL, about 1300 hr*ng/mL, about 1325 hr*ng/mL, about 1350 hr*ng/mL, about 1375 hr*ng/mL, about 1400 hr*ng/mL, about 1425 hr*ng/mL, about 1450 hr*ng/mL, about 1475 hr*ng/mL, about 1500 hr*ng/mL, about 1525 hr*ng/mL, about 1550 hr*ng/mL, about 1575 hr*ng/mL, about 1600 hr*ng/mL, about 1625 hr*ng/mL, about 1650 hr*ng/mL, about 1675 hr*ng/mL, about 1700 hr*ng/mL, about 1725 hr*ng/mL, about 1750 hr*ng/mL, about 1775 hr*ng/mL, about 1800 hr*ng/mL, about 1825 hr*ng/mL, about 1850 hr*ng/mL, about 1875 hr*ng/mL, about 1900 hr*ng/mL, about 1925 hr*ng/mL, about 1950 hr*ng/mL, about 1975 hr*ng/mL, about 2000 hr*ng/mL, about 2025 hr*ng/mL, about 2050 hr*ng/mL, about 2075 hr*ng/mL, about 2100 hr*ng/mL, about 2125 hr*ng/mL, about 2150 hr*ng/mL, about 2175 hr*ng/mL, about 2200 hr*ng/mL, about 2225 hr*ng/mL, about 2250 hr*ng/mL, about 2275 hr*ng/mL, about 2300 hr*ng/mL, about 2325 hr*ng/mL, about 2350 hr*ng/mL, about 2375 hr*ng/mL, about 2400 hr*ng/mL, about 2425 hr*ng/mL, about 2450 hr*ng/mL, about 2475 hr*ng/mL, about 2500 hr*ng/mL, about 2525 hr*ng/mL, about 2550 hr*ng/mL, about 2575 hr*ng/mL, about 2600 hr*ng/mL, about 2625 hr*ng/mL, about 2650 hr*ng/mL, about 2675 hr*ng/mL, about 2700 hr*ng/mL, about 2725 hr*ng/mL, about 2750 hr*ng/mL, about 2775 hr*ng/mL, about 2800 hr*ng/mL, inclusive of all values and subranges therebetween.

In some embodiments, the solid dosage forms described herein provide an average Cₘₐₓ within about 80%-125% of the range of about 29.70±8.61 ng/mL, after a single dose of 200 mg of AmpB, e.g., about 14.5 ng/mL, about 15 ng/mL, about 15.5 ng/mL, about 16 ng/mL, about 16.5 ng/mL, about 17 ng/mL, about 17.5 ng/mL, about 18 ng/mL, about 18.5 ng/mL, about 19 ng/mL, about 19.5 ng/mL, about 20 ng/mL, about 20.5 ng/mL, about 21 ng/mL, about 21.5 ng/mL, about 22 ng/mL, about 22.5 ng/mL, about 23 ng/mL, about 23.5 ng/mL, about 24 ng/mL, about 24.5 ng/mL, about 25 ng/mL, about 25.5 ng/mL, about 30 ng/mL, about 30.5 ng/mL, about 31 ng/mL, about 31.5 ng/mL, about 32 ng/mL, about 32.5 ng/mL, about 33 ng/mL, about 33.5 ng/mL, about 34 ng/mL, about 34.5 ng/mL, about 35 ng/mL, about 35.5 ng/mL, about 36 ng/mL, about 36.5 ng/mL, about 37 ng/mL, about 37.5 ng/mL, about 38 ng/mL, about 38.5 ng/mL, about 39 ng/mL, about 39.5 ng/mL, about 40 ng/mL, about 40.5 ng/mL, about 41 ng/mL, about 41.5 ng/mL, about 42 ng/mL, about 42.5 ng/mL, about 43 ng/mL, about 43.5 ng/mL, about 44 ng/mL, about 44.5 ng/mL, about 45 ng/mL, about 45.5 ng/mL, about 46 ng/mL, about 46.5 ng/mL, about 47 ng/mL, about 47.5 ng/mL, and about 48 ng/mL, inclusive of all values and subranges therebetween.

In some embodiments, the solid dosage forms described herein provide an average Tmax within about 80%-125% of the range of about 6.33±0.82 hr after a single dose of 200 mg of AmpB, e.g., about 3.7 hr, about 3.8 hr, about 3.9 hr, about 4 hr, about 4.1 hr, about 4.2 hr, about 4.3 hr, about 4.4 hr, about 4.5 hr, about 4.6 hr, about 4.7 hr, about 4.8 hr, about 4.9 hr, about 5 hr, about 5.1 hr, about 5.2 hr, about 5.3 hr, about 5.4 hr, about 5.5 hr, about 5.6 hr, about 5.7 hr, about 5.8 hr, about 5.9 hr about 6 hr, about 6.1 hr, about 6.2 hr, about 6.3 hr, about 6.4 hr, about 6.5 hr, about 6.6 hr, about 6.7 hr, about 6.8 hr, about 6.9 hr, about 7 hr, about 7.1 hr, about 7.2 hr, about 7.3 hr, about 7.4 hr, about 7.5 hr, about 7.6 hr, about 7.7 hr, about 7.8 hr, about 7.9 hr, about 8 hr, about 8.1 hr, about 8.2 hr, about 8.3 hr, about 8.4 hr, and about 8.5 hr, inclusive of all values and subranges therebetween.

In some embodiments, the solid dosage forms described herein provide an average AUC₀₋ₜ within about 80%-125% of the range of 1031.10±281.31 hr*ng/mL after a single dose of 200 mg of AmpB, e.g., about 400 hr*ng/mL, about 425 hr*ng/mL, about 450 hr*ng/mL, about 475 hr*ng/mL, about 500 hr*ng/mL, about 525 hr*ng/mL, about 550 hr*ng/mL, about 575 hr*ng/mL, about 600 hr*ng/mL, about 625 hr*ng/mL, about 650 hr*ng/mL, about 675 hr*ng/mL, about 700 hr*ng/mL, about 725 hr*ng/mL, about 750 hr*ng/mL, about 775 hr*ng/mL, about 800 hr*ng/mL, about 825 hr*ng/mL, about 850 hr*ng/mL, about 875 hr*ng/mL, about 900 hr*ng/mL, about 925 hr*ng/mL, about 950 hr*ng/mL, about 975 hr*ng/mL, about 1000 hr*ng/mL, about 1025 hr*ng/mL, about 1050 hr*ng/mL, about 1075 hr*ng/mL, about 1100 hr*ng/mL, about 1125 hr*ng/mL, about 1150 hr*ng/mL, about 1175 hr*ng/mL, about 1200 hr*ng/mL, about 1225 hr*ng/mL, about 1250 hr*ng/mL, about 1275 hr*ng/mL, about 1300 hr*ng/mL, about 1325 hr*ng/mL, about 1350 hr*ng/mL, about 1375 hr*ng/mL, about 1400 hr*ng/mL, about 1425 hr*ng/mL, about 14500 hr*ng/mL, about 1475 hr*ng/mL, about 1500 hr*ng/mL, about 1525 hr*ng/mL, about 1550 hr*ng/mL, about 1575 hr*ng/mL, about 1600 hr*ng/mL, about 1625 hr*ng/mL, about 1650 hr*ng/mL, about 1675 hr*ng/mL, about 1700 hr*ng/mL, about 1725 hr*ng/mL, about 1750 hr*ng/mL, about 1775 hr*ng/mL, about 1800 hr*ng/mL, about 1825 hr*ng/mL, about 1850 hr*ng/mL, about 1875 hr*ng/mL, about 1900 hr*ng/mL, about 1925 hr*ng/mL, about 1950 hr*ng/mL, about 1975 hr*ng/mL, and about 2000 hr*ng/mL, inclusive of all values and subranges therebetween.

In some embodiments, the solid dosage forms described herein provide an average AUC_{0-inf} within about 80%-125% of the range of about 1083.35±269.57 hr*ng/mL after a single dose of 200 mg of AmpB, e.g., about 400 hr*ng/mL, about 425 hr*ng/mL, about 450 hr*ng/mL, about 475 hr*ng/mL, about 500 hr*ng/mL, about 525 hr*ng/mL, 550 hr*ng/mL, about 575 hr*ng/mL, about 600 hr*ng/mL, about 625 hr*ng/mL, about 650 hr*ng/mL, about 675 hr*ng/mL, about 700 hr*ng/mL, about 725 hr*ng/mL, about 750 hr*ng/mL, about 775 hr*ng/mL, about 800 hr*ng/mL, about 825 hr*ng/mL, about 850 hr*ng/mL, about 875 hr*ng/mL, about 900 hr*ng/mL, about 925 hr*ng/mL, about 950 hr*ng/mL, about 975 hr*ng/mL, about 1000 hr*ng/mL, about 1025 hr*ng/mL, about 1050 hr*ng/mL, about 1075 hr*ng/mL, about 1100 hr*ng/mL, about 1125 hr*ng/mL, about 1150 hr*ng/mL, about 1175 hr*ng/mL, about 1200 hr*ng/mL, about 1225 hr*ng/mL, about 1250 hr*ng/mL, about 1275 hr*ng/mL, about 1300 hr*ng/mL, about 1325 hr*ng/mL, about 1350 hr*ng/mL, about 1375 hr*ng/mL, about 1400 hr*ng/mL, about 1425 hr*ng/mL, about 1450 hr*ng/mL, about 1475 hr*ng/mL, about 1500 hr*ng/mL, about 1525 hr*ng/mL, about 1550 hr*ng/mL, about 1575 hr*ng/mL, about 1600 hr*ng/mL, about 1625 hr*ng/mL, about 1650 hr*ng/mL, about 1675 hr*ng/mL, about 1700 hr*ng/mL, about 1725 hr*ng/mL, 1750 hr*ng/mL, about 1775 hr*ng/mL, about 1800 hr*ng/mL, about 1825 hr*ng/mL, about 1850 hr*ng/mL, about 1875 hr*ng/mL, and about 1900 hr*ng/mL, inclusive of all values and subranges therebetween.

In some embodiments, the solid dosage forms described herein provide an average Cₘₐₓ within about 80%-125% of the range of about 29.04±7.91 ng/mL, after a single dose of 400 mg of AmpB, e.g., about 14.5 ng/mL, about 15 ng/mL, about 15.5 ng/mL, about 16 ng/mL, about 16.5 ng/mL, about 17 ng/mL, about 17.5 ng/mL, about 18 ng/mL, about 18.5 ng/mL, about 19 ng/mL, about 19.5 ng/mL, about 20 ng/mL, about 20.5 ng/mL, about 21 ng/mL, about 21.5 ng/mL, about 22 ng/mL, about 22.5 ng/mL, about 23 ng/mL, about 23.5 ng/mL, about 24 ng/mL, about 24.5 ng/mL, about 25 ng/mL, about 25.5 ng/mL, about 30 ng/mL, about 30.5 ng/mL, about 31 ng/mL, about 31.5 ng/mL, about 32 ng/mL, about 32.5 ng/mL, about 33 ng/mL, about 33.5 ng/mL, about 34 ng/mL, about 34.5 ng/mL, about 35 ng/mL, about 35.5 ng/mL, about 36 ng/mL, about 36.5 ng/mL, about 37 ng/mL, about 37.5 ng/mL, about 38 ng/mL, about 38.5 ng/mL, about 39 ng/mL, about 39.5 ng/mL, about 40 ng/mL, about 40.5 ng/mL, about 41 ng/mL, about 41.5 ng/mL, about 42 ng/mL, about 42.5 ng/mL, about 43 ng/mL, about 43.5 ng/mL, about 44 ng/mL, about 44.5 ng/mL, about 45 ng/mL, about 45.5 ng/mL, about 46 ng/mL, and about 46.5 ng/mL, inclusive of all values and subranges therebetween.

In some embodiments, the solid dosage forms described herein provide an average Tₘₐₓ within about 80%-125% of the range of about 7.33±2.07 hr after a single dose of 400 mg of AmpB, e.g., about 3.7 hr, about 3.8 hr, about 3.9 hr, about 4 hr, about 4.1 hr, about 4.2 hr, about 4.3 hr, about 4.4 hr, about 4.5 hr, about 4.6 hr, about 4.7 hr, about 4.8 hr, about 4.9 hr, about 5 hr, about 5.1 hr, about 5.2 hr, about 5.3 hr, about 5.4 hr, about 5.5 hr, about 5.6 hr, about 5.7 hr, about 5.8 hr, about 5.9 hr about 6 hr, about 6.1 hr, about 6.2 hr, about 6.3 hr, about 6.4 hr, about 6.5 hr, about 6.6 hr, about 6.7 hr, about 6.8 hr, about 6.9 hr, about 7 hr, about 7.1 hr, about 7.2 hr, about 7.3 hr, about 7.4 hr, about 7.5 hr, about 7.6 hr, about 7.7 hr, about 7.8 hr, about 7.9 hr, about 8 hr, about 8.1 hr, about 8.2 hr, about 8.3 hr, about 8.4 hr, about 8.5 hr, about 8.6 hr, about 8.7 hr, about 8.8 hr, about 8.9 hr, about 9 hr, about 9.1 hr, about 9.2 hr, about 9.3 hr, about 9.4 hr, about 9.5 hr, about 9.6 hr, about 9.7 hr, about 9.8 hr, about 9.9 hr, about 10 hr, about 10.1 hr, about 10.2 hr, about 10.3 hr, about 10.4 hr, about 10.5 hr, about 10.6 hr, about 10.7 hr, about 10.8 hr, about 10.9 hr, about 10 hr, about 10.1 hr, about 10.2 hr, about 10.3 hr, about 10.4 hr, about 10.5 hr, about 10.6 hr, about 10.7 hr, about 10.8 hr, about 10.9 hr, about 11 hr, about 11.1 hr, about 11.2 hr, about 11.3 hr, about 11.4 hr, and about 11.5 hr inclusive of all values and subranges therebetween.

In some embodiments, the solid dosage forms described herein provide an average AUC₀₋ₜ within about 80%-125% of the range of about 2093.35±1583.16 hr*ng/mL after a single dose of 400 mg of AmpB, e.g., about 300 hr*ng/mL, about 325 hr*ng/mL, about 350 hr*ng/mL, about 375 hr*ng/mL, about 400 hr*ng/mL, about 425 hr*ng/mL, about 450 hr*ng/mL, about 475 hr*ng/mL, about 500 hr*ng/mL, about 525 hr*ng/mL, about 550 hr*ng/mL, about 575 hr*ng/mL, about 600 hr*ng/mL, about 625 hr*ng/mL, about 650 hr*ng/mL, about 675 hr*ng/mL, about 700 hr*ng/mL, about 725 hr*ng/mL, about 750 hr*ng/mL, about 775 hr*ng/mL, about 800 hr*ng/mL, about 825 hr*ng/mL, about 850 hr*ng/mL, about 875 hr*ng/mL, about 900 hr*ng/mL, about 925 hr*ng/mL, about 950 hr*ng/mL, about 975 hr*ng/mL, about 1000 hr*ng/mL, about 1025 hr*ng/mL, about 1050 hr*ng/mL, about 1075 hr*ng/mL, about 1100 hr*ng/mL, about 1125 hr*ng/mL, about 1150 hr*ng/mL, about 1175 hr*ng/mL, about 1200 hr*ng/mL, about 1225 hr*ng/mL, about 1250 hr*ng/mL, about 1275 hr*ng/mL, about 1300 hr*ng/mL, about 1325 hr*ng/mL, about 1350 hr*ng/mL, about 1375 hr*ng/mL, about 1400 hr*ng/mL, about 1425 hr*ng/mL, about 1450 hr*ng/mL, about 1475 hr*ng/mL, about 1500 hr*ng/mL, about 1525 hr*ng/mL, about 1550 hr*ng/mL, about 1575 hr*ng/mL, about 1600 hr*ng/mL, about 1625 hr*ng/mL, about 1650 hr*ng/mL, about 1675 hr*ng/mL, about 1700 hr*ng/mL, about 1725 hr*ng/mL, about 1750 hr*ng/mL, about 1775 hr*ng/mL, about 1800 hr*ng/mL, about 1825 hr*ng/mL, about 1850 hr*ng/mL, about 1875 hr*ng/mL, about 1900 hr*ng/mL, about 1925 hr*ng/mL, about 1950 hr*ng/mL, about 1975 hr*ng/mL, about 2000 hr*ng/mL, about 2025 hr*ng/mL, about 2050 hr*ng/mL, about 2075 hr*ng/mL, about 2100 hr*ng/mL, about 2125 hr*ng/mL, about 2150 hr*ng/mL, about 2175 hr*ng/mL, about 2200 hr*ng/mL, about 2225 hr*ng/mL, about 2250 hr*ng/mL, about 2275 hr*ng/mL, about 2300 hr*ng/mL, about 2325 hr*ng/mL, about 2350 hr*ng/mL, about 2375 hr*ng/mL, about 2400 hr*ng/mL, about 2425 hr*ng/mL, about 2450 hr*ng/mL, about 2475 hr*ng/mL, about 2500 hr*ng/mL, about 2525 hr*ng/mL, about 2550 hr*ng/mL, about 2575 hr*ng/mL, about 2600 hr*ng/mL, about 2625 hr*ng/mL, about 2650 hr*ng/mL, about 2675 hr*ng/mL, about 2700 hr*ng/mL, about 2725 hr*ng/mL, about 2750 hr*ng/mL, about 2775 hr*ng/mL, about 2800 hr*ng/mL about 2825 hr*ng/mL, about 2850 hr*ng/mL, about 2875 hr*ng/mL, about 2900 hr*ng/mL, about 2925 hr*ng/mL, about 2950 hr*ng/mL, about 2975 hr*ng/mL, about 3000 hr*ng/mL, about 3025 hr*ng/mL, about 3050 hr*ng/mL, about 3075 hr*ng/mL, about 3100 hr*ng/mL, about 3125 hr*ng/mL, about 3150 hr*ng/mL, about 3175 hr*ng/mL, about 3200 hr*ng/mL, about 3225 hr*ng/mL, about 3250 hr*ng/mL, about 3275 hr*ng/mL, about 3300 hr*ng/mL, about 3325 hr*ng/mL, about 3350 hr*ng/mL, about 3375 hr*ng/mL, about 3400 hr*ng/mL, about 3425 hr*ng/mL, about 3450 hr*ng/mL, about 3475 hr*ng/mL, about 3500 hr*ng/mL, about 3525 hr*ng/mL, about 3550 hr*ng/mL, about 3575 hr*ng/mL, about 3600 hr*ng/mL, about 3625 hr*ng/mL, about 3650 hr*ng/mL, about 3675 hr*ng/mL, about 3700 hr*ng/mL, about 3725 hr*ng/mL, about 3750 hr*ng/mL, about 3775 hr*ng/mL, about 3800 hr*ng/mL, 3825 hr*ng/mL, about 3850 hr*ng/mL, about 3875 hr*ng/mL, about 3900 hr*ng/mL, about 3925 hr*ng/mL, about 3950 hr*ng/mL, about 3975 hr*ng/mL, about 4000 hr*ng/mL, about 4025 hr*ng/mL, about 4050 hr*ng/mL, about 4075 hr*ng/mL, about 4100 hr*ng/mL, about 4125 hr*ng/mL, about 4150 hr*ng/mL, about 4175 hr*ng/mL, about 4200 hr*ng/mL, about 4225 hr*ng/mL, about 4250 hr*ng/mL, about 4275 hr*ng/mL, about 4300 hr*ng/mL, about 4325 hr*ng/mL, about 4350 hr*ng/mL, about 4375 hr*ng/mL, about 4400 hr*ng/mL, about 4425 hr*ng/mL, about 4450 hr*ng/mL, about 4475 hr*ng/mL, about 4500 hr*ng/mL, about 4525 hr*ng/mL, about 4550 hr*ng/mL, about 4575 hr*ng/mL, about 4600 hr*ng/mL, about 4625 hr*ng/mL, about 4650 hr*ng/mL, about 4675 hr*ng/mL, about 4700 hr*ng/mL, about 4725 hr*ng/mL, about 4750 hr*ng/mL, about 4775 hr*ng/mL, about 4800 hr*ng/mL, 4825 hr*ng/mL, about 4850 hr*ng/mL, about 4875 hr*ng/mL, about 4900 hr*ng/mL, about 4925 hr*ng/mL, about 4950 hr*ng/mL, about 4975 hr*ng/mL, and about 5000 hr*ng/mL, inclusive of all values and subranges therebetween.

In some embodiments, the solid dosage forms described herein provide an average AUC0-inf within about 80%-125% of the range of about 4385.39 hr*ng/mL to about 6887.45 hr*ng/mL after a single dose of 400 mg of AmpB, e.g., about 75 hr*ng/mL, about 100 hr*ng/mL, about 125 hr*ng/mL, about 150 hr*ng/mL, about 175 hr*ng/mL, about 200 hr*ng/mL, about 300 hr*ng/mL, about 400 hr*ng/mL, about 500 hr*ng/mL, about 600 hr*ng/mL, about 700 hr*ng/mL, about 800 hr*ng/mL, about 900 hr*ng/mL, about 1000 hr*ng/mL, about 1100 hr*ng/mL, about 1200 hr*ng/mL, about 1300 hr*ng/mL, about 1400 hr*ng/mL, about 1500 hr*ng/mL, about 1600 hr*ng/mL, about 1700 hr*ng/mL, about 1800 hr*ng/mL, about 1900 hr*ng/mL, about 2000 hr*ng/mL, about 2100 hr*ng/mL, about 2200 hr*ng/mL, about 2300 hr*ng/mL, about 2400 hr*ng/mL, about 2500 hr*ng/mL, about 2600 hr*ng/mL, about 2700 hr*ng/mL, about 2800 hr*ng/mL about 2900 hr*ng/mL, about 3000 hr*ng/mL, about 3100 hr*ng/mL, about 3200 hr*ng/mL, about 3300 hr*ng/mL, about 3400 hr*ng/mL, about 3500 hr*ng/mL, about 3600 hr*ng/mL, about 3700 hr*ng/mL, about 3800 hr*ng/mL, about 3900 hr*ng/mL, about 4000 hr*ng/mL, about 4100 hr*ng/mL, about 4200 hr*ng/mL, about 4300 hr*ng/mL, about 4400 hr*ng/mL, about 4500 hr*ng/mL, about 4600 hr*ng/mL, about 4700 hr*ng/mL, about 4800 hr*ng/mL, about 4900 hr*ng/mL, about 5000 hr*ng/mL, about 5100 hr*ng/mL, about 5200 hr*ng/mL, about 5300 hr*ng/mL, about 5400 hr*ng/mL, about 3500 hr*ng/mL, about 5600 hr*ng/mL, about 5700 hr*ng/mL, about 5800 hr*ng/mL, about 5900 hr*ng/mL, about 6000 hr*ng/mL, about 6100 hr*ng/mL, about 6200 hr*ng/mL, about 6300 hr*ng/mL, about 6400 hr*ng/mL, about 6500 hr*ng/mL, about 6600 hr*ng/mL, about 6700 hr*ng/mL, about 6800 hr*ng/mL, about 6900 hr*ng/mL, about 7000 hr*ng/mL, about 7100 hr*ng/mL, about 7200 hr*ng/mL, about 7300 hr*ng/mL, about 7400 hr*ng/mL, about 7500 hr*ng/mL, about 7600 hr*ng/mL, about 7700 hr*ng/mL, about 7800 hr*ng/mL, about 7900 hr*ng/mL, about 8000 hr*ng/mL, about 8100 hr*ng/mL, about 8200 hr*ng/mL, about 8300 hr*ng/mL, about 8400 hr*ng/mL, about 8500 hr*ng/mL, about 8600 hr*ng/mL, about 8700 hr*ng/mL, about 8800 hr*ng/mL, about 8900 hr*ng/mL, about 9000 hr*ng/mL, about 9100 hr*ng/mL, about 9200 hr*ng/mL, about 9300 hr*ng/mL, about 9400 hr*ng/mL, about 9500 hr*ng/mL, about 9600 hr*ng/mL, about 9700 hr*ng/mL, about 9800 hr*ng/mL, about 9900 hr*ng/mL, about 10000 hr*ng/mL, about 10100 hr*ng/mL, about 10200 hr*ng/mL, about 10300 hr*ng/mL, about 10400 hr*ng/mL, about 10500 hr*ng/mL, about 10600 hr*ng/mL, about 10700 hr*ng/mL, about 10800 hr*ng/mL, about 10900 hr*ng/mL, about 11000 hr*ng/mL, about 11100 hr*ng/mL, about 11200 hr*ng/mL, about 11300 hr*ng/mL, about 11400 hr*ng/mL, about 11500 hr*ng/mL, about 11600 hr*ng/mL, about 11700 hr*ng/mL, about 11800 hr*ng/mL, about 11900 hr*ng/mL, about 12000 hr*ng/mL, about 12100 hr*ng/mL, about 12200 hr*ng/mL, about 12300 hr*ng/mL, about 12400 hr*ng/mL, about 12500 hr*ng/mL, about 12600 hr*ng/mL, about 12700 hr*ng/mL, about 12800 hr*ng/mL, about 12900 hr*ng/mL, about 13000 hr*ng/mL, about 13100 hr*ng/mL, about 13200 hr*ng/mL, about 13300 hr*ng/mL, about 13400 hr*ng/mL, about 13500 hr*ng/mL, about 13600 hr*ng/mL, about 13700 hr*ng/mL, about 13800 hr*ng/mL, about 13900 hr*ng/mL, about 14000 hr*ng/mL, about 15100 hr*ng/mL, about 15200 hr*ng/mL, about 15300 hr*ng/mL, about 15400 hr*ng/mL, about 15500 hr*ng/mL, about 15600 hr*ng/mL, about 15700 hr*ng/mL, about 15800 hr*ng/mL, about 15900 hr*ng/mL, about 16000 hr*ng/mL, about 16100 hr*ng/mL, about 16200 hr*ng/mL, about 16300 hr*ng/mL, about 16400 hr*ng/mL, about 16500 hr*ng/mL, about 16600 hr*ng/mL, about 16700 hr*ng/mL, about 16800 hr*ng/mL, about 16900 hr*ng/mL, about 17000 hr*ng/mL, about 17100 hr*ng/mL, about 17200 hr*ng/mL, about 17300 hr*ng/mL, about 17400 hr*ng/mL, about 17500 hr*ng/mL, about 17600 hr*ng/mL, about 17700 hr*ng/mL, about 17800 hr*ng/mL, about 17900 hr*ng/mL, about 18000 hr*ng/mL, about 18100 hr*ng/mL, about 18200 hr*ng/mL, about 18300 hr*ng/mL, about 18400 hr*ng/mL, about 18500 hr*ng/mL, about 18600 hr*ng/mL, about 18700 hr*ng/mL, about 18800 hr*ng/mL, about 18900 hr*ng/mL, about 19000 hr*ng/mL, about 19100 hr*ng/mL, about 19200 hr*ng/mL, about 19300 hr*ng/mL, about 19400 hr*ng/mL, about 19500 hr*ng/mL, about 19600 hr*ng/mL, about 19700 hr*ng/mL, about 19800 hr*ng/mL, about 19900 hr*ng/mL, and about 20000 hr*ng/mL, inclusive of all values and subranges therebetween.

In some embodiments, the solid dosage forms described herein provide an average Cₘₐₓ within about 80%-125% of the range of about 36.65±5.42 ng/mL, after a single dose within 800 mg of AmpB, e.g., about 17 ng/mL, about 17.5 ng/mL, about 18 ng/mL, about 18.5 ng/mL, about 19 ng/mL, about 19.5 ng/mL, about 20 ng/mL, about 20.5 ng/mL, about 21 ng/mL, about 21.5 ng/mL, about 22 ng/mL, about 22.5 ng/mL, about 23 ng/mL, about 23.5 ng/mL, about 24 ng/mL, about 24.5 ng/mL, about 25 ng/mL, about 25.5 ng/mL, about 30 ng/mL, about 30.5 ng/mL, about 31 ng/mL, about 31.5 ng/mL, about 32 ng/mL, about 32.5 ng/mL, about 33 ng/mL, about 33.5 ng/mL, about 34 ng/mL, about 34.5 ng/mL, about 35 ng/mL, about 35.5 ng/mL, about 36 ng/mL, about 36.5 ng/mL, about 37 ng/mL, about 37.5 ng/mL, about 38 ng/mL, about 38.5 ng/mL, about 39 ng/mL, about 39.5 ng/mL, about 40 ng/mL, about 40.5 ng/mL, about 41 ng/mL, about 41.5 ng/mL, about 42 ng/mL, about 42.5 ng/mL, about 43 ng/mL, about 43.5 ng/mL, about 44 ng/mL, about 44.5 ng/mL, about 45 ng/mL, about 45.5 ng/mL, about 46 ng/mL, about 46.5 ng/mL, about 47 ng/mL, about 47.5 ng/mL, about 48 ng/mL, about 48.5 ng/mL, about 49 ng/mL, about 49.5 ng/mL, about 50 ng/mL, about 50.5 ng/mL, about 51 ng/mL, about 51.5 ng/mL, about 52 ng/mL, about 52.5 ng/mL, about 53 ng/mL, about 53.5 ng/mL, about 54 ng/mL, about 54.5 ng/mL, about 55 ng/mL, about 55.5 ng/mL, about 56 ng/mL, and about 56.5 ng/mL, about 57 ng/mL, about 57.5 ng/mL, about 58 ng/mL, about 58.5 ng/mL, about 59 ng/mL, about 59.5 ng/mL, about 60 ng/mL, inclusive of all values and subranges therebetween.

In some embodiments, the solid dosage forms described herein provide an average Tₘₐₓ within about 80%-125% of the range of about 7.69±1.97 hr after a single dose within 800 mg of AmpB, e.g., about 3.5 hr, about 3.6 hr, about 3.7 hr, about 3.8 hr, about 3.9 hr, about 4 hr, about 4.1 hr, about 4.2 hr, about 4.3 hr, about 4.4 hr, about 4.5 hr, about 4.6 hr, about 4.7 hr, about 4.8 hr, about 4.9 hr, about 5 hr, about 5.1 hr, about 5.2 hr, about 5.3 hr, about 5.4 hr, about 5.5 hr, about 5.6 hr, about 5.7 hr, about 5.8 hr, about 5.9 hr about 6 hr, about 6.1 hr, about 6.2 hr, about 6.3 hr, about 6.4 hr, about 6.5 hr, about 6.6 hr, about 6.7 hr, about 6.8 hr, about 6.9 hr, about 7 hr, about 7.1 hr, about 7.2 hr, about 7.3 hr, about 7.4 hr, about 7.5 hr, about 7.6 hr, about 7.7 hr, about 7.8 hr, about 7.9 hr, about 8 hr, about 8.1 hr, about 8.2 hr, about 8.3 hr, about 8.4 hr, about 8.5 hr, about 8.6 hr, about 8.7 hr, about 8.8 hr, about 8.9 hr, about 9 hr, about 9.1 hr, about 9.2 hr, about 9.3 hr, about 9.4 hr, about 9.5 hr, about 9.6 hr, about 9.7 hr, about 9.8 hr, about 9.9 hr, about 10 hr, about 10.1 hr, about 10.2 hr, about 10.3 hr, about 10.4 hr, about 10.5 hr, about 10.6 hr, about 10.7 hr, about 10.8 hr, about 10.9 hr, about 10 hr, about 10.1 hr, about 10.2 hr, about 10.3 hr, about 10.4 hr, about 10.5 hr, about 10.6 hr, about 10.7 hr, about 10.8 hr, about 10.9 hr, about 11 hr, about 11.1 hr, about 11.2 hr, about 11.3 hr, about 11.4 hr, about 11.5 hr about 11.6 hr, about 11.7 hr, about 11.8 hr, about 11.9 hr, about 12 hr, about 12.1 hr, about 12.2 hr, about 12.3 hr, about 12.4 hr, about 12.5 hr, about 12.6 hr, about 12.7 hr, about 12.8 hr, about 12.9 hr, about 13 hr, about 13.1 hr, about 13.2 hr, about 13.3 hr, about 13.4 hr, and about 13.5 hr, inclusive of all values and subranges therebetween.

In some embodiments, the solid dosage forms described herein provide an average AUC₀₋ₜ within about 80%-125% of the range of about 1350.16±355.58 hr*ng/mL after a single dose within 800 mg of AmpB, e.g., about, about 650 hr*ng/mL, about 675 hr*ng/mL, about 700 hr*ng/mL, about 725 hr*ng/mL, about 750 hr*ng/mL, about 775 hr*ng/mL, about 800 hr*ng/mL, about 825 hr*ng/mL, about 850 hr*ng/mL, about 875 hr*ng/mL, about 900 hr*ng/mL, about 925 hr*ng/mL, about 950 hr*ng/mL, about 975 hr*ng/mL, about 1000 hr*ng/mL, about 1025 hr*ng/mL, about 1050 hr*ng/mL, about 1075 hr*ng/mL, about 1100 hr*ng/mL, about 1125 hr*ng/mL, about 1150 hr*ng/mL, about 1175 hr*ng/mL, about 1200 hr*ng/mL, about 1225 hr*ng/mL, about 1250 hr*ng/mL, about 1275 hr*ng/mL, about 1300 hr*ng/mL, about 1325 hr*ng/mL, about 1350 hr*ng/mL, about 1375 hr*ng/mL, about 1400 hr*ng/mL, about 1425 hr*ng/mL, about 1450 hr*ng/mL, about 1475 hr*ng/mL, about 1500 hr*ng/mL, about 1525 hr*ng/mL, about 1550 hr*ng/mL, about 1575 hr*ng/mL, about 1600 hr*ng/mL, about 1625 hr*ng/mL, about 1650 hr*ng/mL, about 1675 hr*ng/mL, about 1700 hr*ng/mL, about 1725 hr*ng/mL, about 1750 hr*ng/mL, about 1775 hr*ng/mL, about 1800 hr*ng/mL, about 1825 hr*ng/mL, about 1850 hr*ng/mL, about 1875 hr*ng/mL, about 1900 hr*ng/mL, about 1925 hr*ng/mL, about 1950 hr*ng/mL, about 1975 hr*ng/mL, about 2000 hr*ng/mL, about 2025 hr*ng/mL, about 2050 hr*ng/mL, about 2075 hr*ng/mL, about 2100 hr*ng/mL, about 2125 hr*ng/mL, about 2150 hr*ng/mL, about 2175 hr*ng/mL, about 2200 hr*ng/mL, about 2225 hr*ng/mL, about 2250 hr*ng/mL, about 2275 hr*ng/mL, about 2300 hr*ng/mL, about 2325 hr*ng/mL, about 2350 hr*ng/mL, about 2375 hr*ng/mL, about 2400 hr*ng/mL, about 2425 hr*ng/mL, about 2450 hr*ng/mL, about 2475 hr*ng/mL, and about 2500 hr*ng/mL, inclusive of all values and subranges therebetween.

In some embodiments, the solid dosage forms described herein provide an average AUC_{0-inf} within about 80%-125% of the range of about 1373.76±363.07 hr*ng/mL after a single dose within 800 mg of AmpB, e.g., about 500 hr*ng/mL, about 525 hr*ng/mL, about 550 hr*ng/mL, about 575 hr*ng/mL, about 600 hr*ng/mL, about 625 hr*ng/mL, about 650 hr*ng/mL, about 675 hr*ng/mL, about 700 hr*ng/mL, about 725 hr*ng/mL, about 750 hr*ng/mL, about 775 hr*ng/mL, about 800 hr*ng/mL, about 825 hr*ng/mL, about 850 hr*ng/mL, about 875 hr*ng/mL, about 900 hr*ng/mL, about 925 hr*ng/mL, about 950 hr*ng/mL, about 975 hr*ng/mL, about 1000 hr*ng/mL, about 1025 hr*ng/mL, about 1050 hr*ng/mL, about 1075 hr*ng/mL, about 1100 hr*ng/mL, about 1125 hr*ng/mL, about 1150 hr*ng/mL, about 1175 hr*ng/mL, about 1200 hr*ng/mL, about 1225 hr*ng/mL, about 1250 hr*ng/mL, about 1275 hr*ng/mL, about 1300 hr*ng/mL, about 1325 hr*ng/mL, about 1350 hr*ng/mL, about 1375 hr*ng/mL, about 1400 hr*ng/mL, about 1425 hr*ng/mL, about 1450 hr*ng/mL, about 1475 hr*ng/mL, about 1500 hr*ng/mL, about 1525 hr*ng/mL, about 1550 hr*ng/mL, about 1575 hr*ng/mL, about 1600 hr*ng/mL, about 1625 hr*ng/mL, about 1650 hr*ng/mL, about 1675 hr*ng/mL, about 1700 hr*ng/mL, about 1725 hr*ng/mL, about 1750 hr*ng/mL, about 1775 hr*ng/mL, about 1800 hr*ng/mL, about 1825 hr*ng/mL, about 1850 hr*ng/mL, about 1875 hr*ng/mL, about 1900 hr*ng/mL, about 1925 hr*ng/mL, about 1950 hr*ng/mL, about 1975 hr*ng/mL, about 2000 hr*ng/mL, about 2025 hr*ng/mL, about 2050 hr*ng/mL, about 2075 hr*ng/mL, about 2100 hr*ng/mL, about 2125 hr*ng/mL, about 2150 hr*ng/mL, about 2175 hr*ng/mL, about 2200 hr*ng/mL, about 2225 hr*ng/mL, about 2250 hr*ng/mL, about 2275 hr*ng/mL, about 2300 hr*ng/mL, about 2325 hr*ng/mL, about 2350 hr*ng/mL, about 2375 hr*ng/mL, about 2400 hr*ng/mL, about 2425 hr*ng/mL, about 2450 hr*ng/mL, about 2475 hr*ng/mL, and about 2500 hr*ng/mL, inclusive of all values and subranges therebetween.

The amphotericin B dosage forms described may be administered according to any suitable dosing regimen which is sufficient to treat a condition in a subject in need thereof. In particular embodiments, the subject is administered an amphotericin B formulation as described herein one or more, two or more, three or more, four or more, five or more, or six or more times, with a duration of time occurring between each provision. In some embodiments, it may be necessary to administer multiple dosage forms at the same time in order to provide the required dose. In particular embodiments, the subject (e.g., a human) is provided with the amphotericin B formulation once, twice, three times, four times, five times, six times, seven times, eight times, nine times, or ten times, with a duration of time between each provision. In particular embodiments, a subject is provided with the amphotericin B formulation about once per day for about four days, about once per day for about five days, about once per day for about six days, or about once per day for about one week. In particular embodiments, a subject is provided with the amphotericin B formulation once a day, twice a day, three times a day or four times a day, e.g., for any of the durations of time described herein. In particular embodiments, the subject is provided with the amphotericin B formulation about once a day, twice a day, three times a day, four times a day, or once every two days for about three days, four days, five days six days, one week, two weeks, three weeks, one month or two months, or longer. In particular embodiments, the days and/or weeks are consecutive. In some embodiments, the amphotericin B dosage forms described herein are formulated for administration once daily.

In some embodiments, the total daily dosage of amphotericin B is an amount in the range of from about 50 mg/day to about 1500 mg/day, e.g., about 100 mg/day, about 150 mg/day, about 200 mg/day, about 250 mg/day, about 200 mg/day, about 250 mg/day, about 300 mg/day, about 350 mg/day, about 400 mg/day, about 450 mg/day, about 500 mg/day, about 550 mg/day, about 600 mg/day, about 650 mg/day, about 700 mg/day, about 750 mg/day, about 800 mg/day, about 850 mg/day, about 900 mg/day, about 950 mg/day, about 1000 mg/day, about 1050 mg/day, about 1100 mg/day, about 1150 mg/day, about 1200 mg/day, about 1250 mg/day, about 1200 mg/day, about 1250 mg/day, about 1300 mg/day, about 1350 mg/day, about 1400 mg/day, about 1450 mg/day, or about 1500 mg/day, inclusive of all values and subranges therein.

In some embodiments, a subject is provided with an amphotericin B formulation disclosed herein multiple times per day. In some such embodiments, amphotericin B is present in the single dosage in an amount in the range of from about 50 mg/day to about 1500 mg/day, e.g., about 100 mg/day, about 150 mg/day, about 200 mg/day, about 250 mg/day, about 200 mg/day, about 250 mg/day, about 300 mg/day, about 350 mg/day, about 400 mg/day, about 450 mg/day, about 500 mg/day, about 550 mg/day, about 600 mg/day, about 650 mg/day, about 700 mg/day, about 750 mg/day, about 800 mg/day, about 850 mg/day, about 900 mg/day, about 950 mg/day, about 1000 mg/day, about 1050 mg/day, about 1100 mg/day, about 1150 mg/day, about 1200 mg/day, about 1250 mg/day, about 1200 mg/day, about 1250 mg/day, about 1300 mg/day, about 1350 mg/day, about 1400 mg/day, about 1450 mg/day, or about 1500 mg/day, inclusive of all values and subranges therein.

In some embodiments, a single dose of the amphotericin B formulations disclosed herein includes multiple dosage forms (e.g., multiple capsules). For example, in some embodiments, a single dose of an amphotericin B formulation can include at least about 1 dosage form, at least about 2 dosage forms, at about least 3 dosage forms, at about least 4 dosage forms, at about least 5 dosage forms, at least about 6 dosage forms, at least about 7 dosage forms, at least about 8 dosage forms, at least about 9 dosage forms, or at least about 10 dosage forms, etc. In other embodiments, a single dose of an amphotericin B formulation include from about 1 dosage form to about 10 dosage forms, e.g., about 2, about 3, about 4, about 5, about 6, about 7, about 8, or about 9 dosage forms, inclusive of all values and subranges therein.

The amphotericin B dosage forms may be administered to treat any infection which is responsive to amphotericin B. In some embodiments, the amphotericin dosage forms described herein may be used to treat infectious diseases, such as fungal infections, human immunodeficiency virus (HIV), and parasitic infections. Infectious diseases treatable by the method and formulations disclosed herein include fungal infections (aspergillosis, blastomycosis, candidiasis, coccidioidomycosis, crytococcosis, histoplasmosis, mucormycosis, paracoccidioidomycosis, and sporotrichosis), visceral leishmaniasis, leishmaniasis, mucocutaneous, cutaneous leishmaniasis, Chagas disease, and Febrile neutropenia. Amphotericin B has been shown to bind to amyloid and prevent the formation of fibrils. Accordingly, the Amphotericin B disclosed herein may be useful for the treatment of diseases associated with fibril formations, such as Alzheimer's disease.

In some embodiments, the disclosure provides methods for treating visceral leishmaniasis comprising orally administering a solid dosage form described herein comprising an effective amount of amphotericin B to a subject in need thereof. In another embodiment, the disclosure provides for a method of treating a fungal infection comprising orally administering a solid dosage form described herein comprising an effective amount of amphotericin B described herein to a subject in need thereof. In particular embodiments, a therapeutically effective amount of amphotericin B is sufficient to achieve a blood plasma level of 0.01 μM to 10 mM, 0.01 μM to 1 mM, 0.01 μM to 100 nM, or 0.01 μM to 10 mM. The therapeutically effective amount of amphotericin B administered can vary depending on the subject and the severity of the condition. In one embodiment, the therapeutically effective amount can range from about 0.01 to about 1000 mg/kg, about 0.1 to about 100 mg/kg, about 0.5 to about 50 mg/kg, about 1 to about 20 mg/kg subject body weight, or about 5 to about 10 mg/kg, e.g., about 5, about 6, about 7, about 8, about 9, or about 10 mg/kg.

In some embodiments, the present disclosure provides for a method of treating an infectious disease (as described above), such as a Lesishmania infection in a patient, comprising administering to the subject a solid dosage form described herein comprising an amount of AmpB in the range of from about 100-800 mg (inclusive of all values and subranges therebetween), wherein the subject has any of the pharmacokinetic parameters described above, as measured after a single dose of about 100-800 mg of AmpB.

More specifically, in some embodiments, the subject has an average Cₘₐₓ within about 80%-125% of the range of from about 21.09 ng/mL to about 42.07 ng/mL, after a single dose of about 100-800 mg of AmpB (inclusive of all values and subranges therebetween), e.g., about 14 ng/mL, about 14.5 ng/mL, about 15 ng/mL, about 15.5 ng/mL, about 16 ng/mL, about 16.5 ng/mL, about 17 ng/mL, about 17.5 ng/mL, about 18 ng/mL, about 18.5 ng/mL, about 19 ng/mL, about 19.5 ng/mL, about 20 ng/mL, about 20.5 ng/mL, about 21 ng/mL, about 21.5 ng/mL, about 22 ng/mL, about 22.5 ng/mL, about 23 ng/mL, about 23.5 ng/mL, about 24 ng/mL, about 24.5 ng/mL, about 25 ng/mL, about 25.5 ng/mL, about 30 ng/mL, about 30.5 ng/mL, about 31 ng/mL, about 31.5 ng/mL, about 32 ng/mL, about 32.5 ng/mL, about 33 ng/mL, about 33.5 ng/mL, about 34 ng/mL, about 34.5 ng/mL, about 35 ng/mL, about 35.5 ng/mL, about 36 ng/mL, about 36.5 ng/mL, about 37 ng/mL, about 37.5 ng/mL, about 38 ng/mL, about 38.5 ng/mL, about 39 ng/mL, about 39.5 ng/mL, about 40 ng/mL, about 40.5 ng/mL, about 41 ng/mL, about 41.5 ng/mL, about 42 ng/mL, about 42.5 ng/mL, about 43 ng/mL, about 43.5 ng/mL, about 44 ng/mL, about 44.5 ng/mL, about 45 ng/mL, about 45.5 ng/mL, about 46 ng/mL, about 46.5 ng/mL, about 47 ng/mL, about 47.5 ng/mL, about 48 ng/mL, about 48.5 ng/mL, about 49 ng/mL, about 49.5 ng/mL, about 50 ng/mL, about 50.5 ng/mL, about 51 ng/mL, about 51.5 ng/mL, about 52 ng/mL, about 52.5 ng/mL, about 53 ng/mL, about 53.5 ng/mL, about 54 ng/mL, about 54.5 ng/mL, about 55 ng/mL, about 55.5 ng/mL, about 56 ng/mL, about 56.5 ng/mL, about 57 ng/mL, about 57.5 ng/mL, about 58 ng/mL, about 58.5 ng/mL, about 59 ng/mL, about 59.5 ng/mL, and about 60 ng/mL inclusive of all values and subranges therebetween.

In some embodiments, the subject has an average Tₘₐₓ within about 80%-125% of the range of from about 5.25 hr to about 9.66 hr after a single dose of about 100 to about 400 mg of AmpB (inclusive of all values and subranges therebetween) e.g., about 3.7 hr, about 3.8 hr, about 3.9 hr, about 4 hr, about 4.1 hr, about 4.2 hr, about 4.3 hr, about 4.4 hr, about 4.5 hr, about 4.6 hr, about 4.7 hr, about 4.8 hr, about 4.9 hr, about 5 hr, about 5.1 hr, about 5.2 hr, about 5.3 hr, about 5.4 hr, about 5.5 hr, about 5.6 hr, about 5.7 hr, about 5.8 hr, about 5.9 hr about 6 hr, about 6.1 hr, about 6.2 hr, about 6.3 hr, about 6.4 hr, about 6.5 hr, about 6.6 hr, about 6.7 hr, about 6.8 hr, about 6.9 hr, about 7 hr, about 7.1 hr, about 7.2 hr, about 7.3 hr, about 7.4 hr, about 7.5 hr, about 7.6 hr, about 7.7 hr, about 7.8 hr, about 7.9 hr, about 8 hr, about 8.1 hr, about 8.2 hr, about 8.3 hr, about 8.4 hr, about 8.5 hr, about 8.6 hr, about 8.7 hr, about 8.8 hr, about 8.9 hr, about 9 hr, about 9.1 hr, about 9.2 hr, about 9.3 hr, about 9.4 hr, about 9.5 hr, about 9.6 hr, about 9.7 hr, about 9.8 hr, about 9.9 hr, about 10 hr, about 10.1 hr, about 10.2 hr, about 10.3 hr, about 10.4 hr, about 10.5 hr, about 10.6 hr, about 10.7 hr, about 10.8 hr, about 10.9 hr, about 10 hr, about 10.1 hr, about 10.2 hr, about 10.3 hr, about 10.4 hr, about 10.5 hr, about 10.6 hr, about 10.7 hr, about 10.8 hr, about 10.9 hr, about 11 hr, about 11.1 hr, about 11.2 hr, about 11.3 hr, about 11.4 hr, about 11.5 hr, about 11.6 hr, about 11.7 hr, about 11.8 hr, about 11.9 hr, about 12 hr, about 12.1 hr, about 12.2 hr, about 12.3 hr, about 12.4 hr, about 12.5 hr, about 12.6 hr, about 12.7 hr, about 12.8 hr, about 12.9 hr, about 13 hr, about 13.1 hr, about 13.2 hr, about 13.3 hr, about 13.4 hr, and about 13.5 hr, inclusive of all values and subranges therebetween.

In some embodiments, the subject has an average AUC₀₋ₜ within about 80%-125% of the range of from about 510.00 hr*ng/mL to about 4779.45 hr*ng/mL after a single dose of about 100-800 mg of AmpB (inclusive of all values and subranges therebetween), e.g., about 450 hr*ng/mL, about 475 hr*ng/mL, about 500 hr*ng/mL, about 525 hr*ng/mL, about 550 hr*ng/mL, about 575 hr*ng/mL, about 600 hr*ng/mL, about 625 hr*ng/mL, about 650 hr*ng/mL, about 675 hr*ng/mL, about 700 hr*ng/mL, about 725 hr*ng/mL, about 750 hr*ng/mL, about 775 hr*ng/mL, about 800 hr*ng/mL, about 825 hr*ng/mL, about 850 hr*ng/mL, about 875 hr*ng/mL, about 900 hr*ng/mL, about 925 hr*ng/mL, about 950 hr*ng/mL, about 975 hr*ng/mL, about 1000 hr*ng/mL, about 1025 hr*ng/mL, about 1050 hr*ng/mL, about 1075 hr*ng/mL, about 1100 hr*ng/mL, about 1125 hr*ng/mL, about 1150 hr*ng/mL, about 1175 hr*ng/mL, about 1200 hr*ng/mL, about 1225 hr*ng/mL, about 1250 hr*ng/mL, about 1275 hr*ng/mL, about 1300 hr*ng/mL, about 1325 hr*ng/mL, about 1350 hr*ng/mL, about 1375 hr*ng/mL, about 1400 hr*ng/mL, about 1425 hr*ng/mL, about 1450 hr*ng/mL, about 1475 hr*ng/mL, about 1500 hr*ng/mL, about 1525 hr*ng/mL, about 1550 hr*ng/mL, about 1575 hr*ng/mL, about 1600 hr*ng/mL, about 1625 hr*ng/mL, about 1650 hr*ng/mL, about 1675 hr*ng/mL, about 1700 hr*ng/mL, about 1725 hr*ng/mL, about 1750 hr*ng/mL, about 1775 hr*ng/mL, about 1800 hr*ng/mL, about 1825 hr*ng/mL, about 1850 hr*ng/mL, about 1875 hr*ng/mL, about 1900 hr*ng/mL, about 1925 hr*ng/mL, about 1950 hr*ng/mL, about 1975 hr*ng/mL, about 2000 hr*ng/mL, about 2025 hr*ng/mL, about 2050 hr*ng/mL, about 2075 hr*ng/mL, about 2100 hr*ng/mL, about 2125 hr*ng/mL, about 2150 hr*ng/mL, and about 2175 hr*ng/mL, inclusive of all values and subranges therebetween.

In some embodiments, the subject has an average AUC_{0-inf} within about 80%-125% of the range of from about 509.84 hr*ng/mL to about 18366.24 hr*ng/mL after a single dose of about 100-800 mg of AmpB (inclusive of all values and subranges therebetween), e.g., about 400 hr*ng/mL, about 500 hr*ng/mL, about 600 hr*ng/mL, about 700 hr*ng/mL, about 800 hr*ng/mL, about 900 hr*ng/mL, about 1000 hr*ng/mL, about 1100 hr*ng/mL, about 1200 hr*ng/mL, about 1300 hr*ng/mL, about 1400 hr*ng/mL, about 1500 hr*ng/mL, about 1600 hr*ng/mL, about 1700 hr*ng/mL, about 1800 hr*ng/mL, about 1900 hr*ng/mL, about 2000 hr*ng/mL, about 2100 hr*ng/mL, about 2200 hr*ng/mL, about 2300 hr*ng/mL, about 2400 hr*ng/mL, about 2500 hr*ng/mL, about 2600 hr*ng/mL, about 2700 hr*ng/mL, about 2800 hr*ng/mL about 2900 hr*ng/mL, about 3000 hr*ng/mL, about 3100 hr*ng/mL, about 3200 hr*ng/mL, about 3300 hr*ng/mL, about 3400 hr*ng/mL, about 3500 hr*ng/mL, about 3600 hr*ng/mL, about 3700 hr*ng/mL, about 3800 hr*ng/mL, about 3900 hr*ng/mL, about 4000 hr*ng/mL, about 4100 hr*ng/mL, about 4200 hr*ng/mL, about 4300 hr*ng/mL, about 4400 hr*ng/mL, about 4500 hr*ng/mL, about 4600 hr*ng/mL, about 4700 hr*ng/mL, about 4800 hr*ng/mL, about 4900 hr*ng/mL, about 5000 hr*ng/mL, about 5100 hr*ng/mL, about 5200 hr*ng/mL, about 5300 hr*ng/mL, about 5400 hr*ng/mL, about 3500 hr*ng/mL, about 5600 hr*ng/mL, about 5700 hr*ng/mL, about 5800 hr*ng/mL, about 5900 hr*ng/mL, about 6000 hr*ng/mL, about 6100 hr*ng/mL, about 6200 hr*ng/mL, about 6300 hr*ng/mL, about 6400 hr*ng/mL, about 6500 hr*ng/mL, about 6600 hr*ng/mL, about 6700 hr*ng/mL, about 6800 hr*ng/mL, about 6900 hr*ng/mL, about 7000 hr*ng/mL, about 7100 hr*ng/mL, about 7200 hr*ng/mL, about 7300 hr*ng/mL, about 7400 hr*ng/mL, about 7500 hr*ng/mL, about 7600 hr*ng/mL, about 7700 hr*ng/mL, about 7800 hr*ng/mL, about 7900 hr*ng/mL, about 8000 hr*ng/mL, about 8100 hr*ng/mL, about 8200 hr*ng/mL, about 8300 hr*ng/mL, about 8400 hr*ng/mL, about 8500 hr*ng/mL, about 8600 hr*ng/mL, about 8700 hr*ng/mL, about 8800 hr*ng/mL, about 8900 hr*ng/mL, about 9000 hr*ng/mL, about 9100 hr*ng/mL, about 9200 hr*ng/mL, about 9300 hr*ng/mL, about 9400 hr*ng/mL, about 9500 hr*ng/mL, about 9600 hr*ng/mL, about 9700 hr*ng/mL, about 9800 hr*ng/mL, about 9900 hr*ng/mL, about 10000 hr*ng/mL, about 10100 hr*ng/mL, about 10200 hr*ng/mL, about 10300 hr*ng/mL, about 10400 hr*ng/mL, about 10500 hr*ng/mL, about 10600 hr*ng/mL, about 10700 hr*ng/mL, about 10800 hr*ng/mL, about 10900 hr*ng/mL, about 11000 hr*ng/mL, about 11100 hr*ng/mL, about 11200 hr*ng/mL, about 11300 hr*ng/mL, about 11400 hr*ng/mL, about 11500 hr*ng/mL, about 11600 hr*ng/mL, about 11700 hr*ng/mL, about 11800 hr*ng/mL, about 11900 hr*ng/mL, about 12000 hr*ng/mL, about 12100 hr*ng/mL, about 12200 hr*ng/mL, about 12300 hr*ng/mL, about 12400 hr*ng/mL, about 12500 hr*ng/mL, about 12600 hr*ng/mL, about 12700 hr*ng/mL, about 12800 hr*ng/mL, about 12900 hr*ng/mL, about 13000 hr*ng/mL, about 13100 hr*ng/mL, about 13200 hr*ng/mL, about 13300 hr*ng/mL, about 13400 hr*ng/mL, about 13500 hr*ng/mL, about 13600 hr*ng/mL, about 13700 hr*ng/mL, about 13800 hr*ng/mL, about 13900 hr*ng/mL, about 14000 hr*ng/mL, about 15100 hr*ng/mL, about 15200 hr*ng/mL, about 15300 hr*ng/mL, about 15400 hr*ng/mL, about 15500 hr*ng/mL, about 15600 hr*ng/mL, about 15700 hr*ng/mL, about 15800 hr*ng/mL, about 15900 hr*ng/mL, about 16000 hr*ng/mL, about 16100 hr*ng/mL, about 16200 hr*ng/mL, about 16300 hr*ng/mL, about 16400 hr*ng/mL, about 16500 hr*ng/mL, about 16600 hr*ng/mL, about 16700 hr*ng/mL, about 16800 hr*ng/mL, about 16900 hr*ng/mL, about 17000 hr*ng/mL, about 17100 hr*ng/mL, about 17200 hr*ng/mL, about 17300 hr*ng/mL, about 17400 hr*ng/mL, about 17500 hr*ng/mL, about 17600 hr*ng/mL, about 17700 hr*ng/mL, about 17800 hr*ng/mL, about 17900 hr*ng/mL, about 18000 hr*ng/mL, about 18100 hr*ng/mL, about 18200 hr*ng/mL, about 18300 hr*ng/mL, about 18400 hr*ng/mL, about 18500 hr*ng/mL, about 18600 hr*ng/mL, about 18700 hr*ng/mL, about 18800 hr*ng/mL, about 18900 hr*ng/mL, about 19000 hr*ng/mL, about 19100 hr*ng/mL, about 19200 hr*ng/mL, about 19300 hr*ng/mL, about 19400 hr*ng/mL, about 19500 hr*ng/mL, about 19600 hr*ng/mL, about 19700 hr*ng/mL, about 19800 hr*ng/mL, about 19900 hr*ng/mL, about 20000 hr*ng/mL, inclusive of all values and subranges therebetween.

In some embodiments, the solid dosage forms of the present disclosure increase the AUC (e.g., AUC₀₋₂₄, AUCₒ₋ₜ, AUC_{0-inf}, etc) by at least about 5% compared to different dosage forms having the same dose of amphotericin B, e.g., by at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, or more.

The solid dosage forms and methods described herein can provide any one of the Cₘₐₓ, any Tₘₐₓ, any AUC₀₋ₜ, or any AUC_{0-inf} disclosed herein. Further, the solid dosage forms and methods described herein can provide any combination of Cₘₐₓ, Tₘₐₓ, AUC₀₋ₜ, or AUC_{0-inf}. That is, the solid dosage forms and methods described herein can provide any combination of any Cₘₐₓ and Tₘₐₓ described herein; any Cₘₐₓ and AUC₀₋ₜ described herein; and Cₘₐₓ and AUC_{0-inf} described herein; any Cₘₐₓ, any AUC₀₋ₜ, and AUC_{0-inf} described herein, and any combinations of these.

### EXAMPLES

### Example 1

### Materials and Methods

Table 1 provides a description of the materials used in the analytical studies described herein. The amphotericin (AmpB) was stored at 2-8° C., protected from light and moisture. Other materials were stored at room temperature (RT).

TABLE 1
Materials

| Material (Trade Name) | Functionality | Lot No. | Supplier |
|---|---|---|---|
| Amphotericin B | API | C00729 | Xellia |
| | | | Pharmaceuticals |
| Ethanol | Solvent | E00400 | Commercial Alcohol |
| Lauroyl polyoxylglycerides | Filler | 136981 | Gattefossé |
| (Gelucire ® 44/14) | | | |
| Glyceryl monooleate | | 147329 | Gattefossé |
| type 40 (Peceol ®) | | | |
| Polyethylene glycol succinate | | 1301080017 | Isochem |
| (Vitamin E TPGS) | | | |
| Microcrystalline cellulose type 101 | | C00465 | Blanver |
| (Tabulose ® 101) | | | |
| Mannitol (Pearlitol ® 160C) | | C00464 | Roquette |
| Silicified microcrystalline cellulose | | C00618 | JRS Pharma |
| (Prosolv ® HD 90) | | | |
| Polyvinyl pyrrolidone (Plasdone ® K-29/32) | Binder | C00605 | Ashland |
| Croscarmellose sodium type A (Solutab ®) | Disintegrant | C00593 | Blanver |
| Colloidal silicon dioxide (Aerosil ® 200) | Glidant | C00449 | Degussa |
| Magnesium stearate (Ligamed ® MF-2-V) | Lubricant | C00124 | Peter Greven |
| Hard gelatin capsule size 0 | — | 70965701 | Capsugel |
| Hard gelatin capsule size 00 | — | C00420 | Capsugel |

Sample Preparation 1
Empty the contents of 2 capsules into a 200 ml low actinic flask.
Add NMP (˜80% of the volume) and sonicate for 15 minutes, with ice pack in the bath.
Shake for 15 minutes.
Mix and allow solution to equilibrate to room temperature.
Dilute to volume with NMP.
Dilute 5 mL of the above solution to 50 mL with Diluent A (25% ammonium acetate solution/25% NMP/50% methanol).
Filter with a 0.45 μm nylon filter, discarding the first 3 ml.

Sample Preparation 2
Empty the contents of 2 capsules into a 200 ml low actinic flask.
Add 50 ml of NMP and sonicate for 15 minutes, with ice pack in the bath.
Shake for 15 minutes.
Add ˜90% of the volume of Diluent B (ammonium acetate solution/methanol 1:2).
Mix well and allow solution to equilibrate to room temperature.
Dilute to volume with Diluent B.
Dilute 5 mL of the above solution to 50 mL with Diluent A (25% ammonium acetate solution/25% NMP/50% methanol).
Filter with a 0.45 μm nylon filter, discarding the first 3 ml.

Sample Preparation 3
Transfer the contents and empty gelatin capsules of 4 capsules into a 500 ml low actinic flask.
Add 125 ml of NMP and sonicate for 30-45 minutes (with ice packs in the bath to minimize heating) until the sample is completely dispersed. Shake vigorously at regular intervals during sonication. Note: The capsule shells remain intact.
Add ˜90% of the volume of Diluent B (ammonium acetate solution/methanol 1:2).
Mix well and allow solution to equilibrate to room temperature (placed in refrigerator to cool quickly).
Dilute to volume with Diluent B.
Dilute 3 mL of the above solution to 25 mL with Diluent A (25% ammonium acetate solution/25% NMP/50% methanol).
Filter with a 0.45 μm nylon filter, discarding the first 3 ml.

Sample Preparation 4
Transfer the contents of 3 capsules into a 500 ml low actinic flask.
Complete to volume with 0.5% SDS in water.
Add a stir bar and stir for a least 90 minutes.
Dilute 8 mL of the above solution to 50 mL with 0.5% SDS in water
Filter with a 0.45 μm nylon filter, discarding the first 3 ml.

### Example 2

### Solid Formulations

Amphotericin B with Gelucire/Peceol/TPGS containing formulations were prepared as shown in Tables 2-4 based on the reference iCo/Wasan liquid formulation in Table 5.

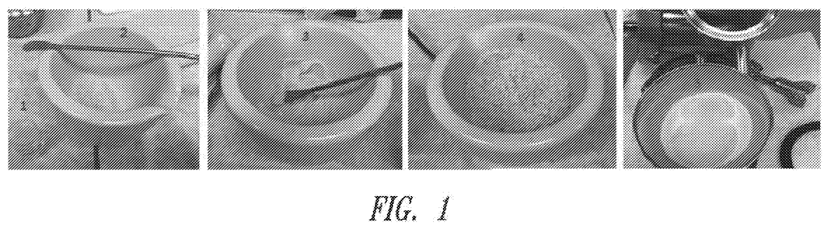

First Gelucire and TPGS were melted and weighed, both in a same container. Peceol was weighed and added to the Gelucire-TPGS mixture. Ethanol was weighed and added to the Gelucire-TPGS-Peceol mixture and mixed using a stirring heating plate at a temperature of about 40° C. until all ingredients dissolved (#1 in FIG. 1). The solution was added to API (#2 in FIG. 1) and mixed for about 5 min using a pestle. This mixture was ‘creamy’ at 25° C. (#3 in FIG. 1). The

Internal phase powder excipients were mixed separately using a V-blender at 25 rpm for 2 min. Both mixtures were mixed using a pestle/mortar for about 5 min. The resulting mixture (#4 in FIG. 1) was placed in an oven at 40° C. for 1-2 h to evaporate ethanol and then removed from oven and kept for about 2 h at 22-25° C. The granules were obtained by milling through a 20 mesh (850 μm) screen. The lubricant (e.g., magnesium stearate) was mixed with granules using a V-blender for 2 min. The final blend (#5 in FIG. 1) was encapsulated into size “0” hard shell gelatin capsules (435 mg/caps). The capsules were filled by volume using tapping/tamping technique.

TABLE 2
Formulation 1

| Item | Ingredient | % w/w | mg/unit | g/batch |
|---|---|---|---|---|
| a | Amphotericin B | 23.0 | 100 | 4.60 |
| b | Mannitol 160C | 34.5 | 150 | 6.90 |
| c | Tabulose 101 | 34.3 | 149 | 6.85 |
| d | Colloidal silicon dioxide | 2.3 | 10 | 0.46 |
| e | TPGS | 0.2 | 1 | 0.05 |
| f | Peceol | 2.3 | 10 | 0.46 |
| g | Gelucire 44/14 | 2.3 | 10 | 0.46 |
| h | Ethanol 100% (evaporated | (30.0) | — | (6.00) |
| | during the process) | | | |
| i | Magnesium stearate | 1.1 | 5 | 0.23 |

| | | | |
|---|---|---|---|
| Total: | 100 | 435 | 20 |

Items a-h are internal phase components, and item i is the external phase component.

TABLE 3
Formulation 2

| Item | Ingredient | % w/w | mg/unit | g/batch |
|---|---|---|---|---|
| a | Amphotericin B | 23.0 | 100 | 4.60 |
| b | Prosolv HD90 | 66.0 | 287 | 13.21 |
| c | Croscarmellose sodium | 5.0 | 22 | 1.00 |
| d | TPGS | 0.2 | 1 | 0.05 |
| e | Peceol | 2.3 | 10 | 0.46 |
| f | Gelucire 44/14 | 2.3 | 10 | 0.46 |
| g | Ethanol 100% (evaporated | (30.0) | — | (6.00) |
| | during the process) | | | |
| h | Magnesium stearate | 1.1 | 5 | 0.23 |

| | | | |
|---|---|---|---|
| Total: | 100 | 435 | 20 |

Items a-g are internal phase components, and item h is the external phase component.

TABLE 4
Formulation 3

| Item | Ingredient | % w/w | mg/unit | g/batch |
|---|---|---|---|---|
| a | Amphotericin B | 23.0 | 100 | 4.60 |
| b | Tabulose 101 | 66.0 | 287 | 13.21 |
| c | Plasdone K-29/32 | 5.0 | 22 | 1.00 |
| d | TPGS | 0.2 | 1 | 0.05 |
| e | Peceol | 2.3 | 10 | 0.46 |
| f | Gelucire 44/14 | 2.3 | 10 | 0.46 |
| g | Ethanol 100% (evaporated | (30.0) | — | (6.00) |
| | during the process) | | | |
| h | Magnesium stearate | 1.1 | 5 | 0.23 |

| | | | |
|---|---|---|---|
| Total: | 100 | 435 | 20 |

Items a-g are internal phase components, and item h is the external phase component.

TABLE 5
iCo/Wasan Formulation compared to Formulations 1-3

| | iCo/Wasan | | Formulation 1 | | Formulation 2 | | Formulation 3 | |
|---|---|---|---|---|---|---|---|---|
| Ingredient | Formulation | | (L268-01016) | | (L268-01017) | | (L268-01018) | |
| Amphotericin B | 150 | mg | 100 | mg | 100 | mg | 100 | mg |
| TPGS | 1.5 | mL | 1 | mg | 1 | mg | 1 | mg |
| Peceol | 14.25 | mL | 10 | mg | 10 | mg | 10 | mg |
| Gelucire 44/14 | 14.25 | mL | 10 | mg | 10 | mg | 10 | mg |

| Ethanol* 100% | (40 mL) | (6 g) | | (6 g) | | (6 g) | |
|---|---|---|---|---|---|---|---|
| Mannitol 160C | — | 150 | mg | — | | — | |
| Tabulose 101 | — | 149 | mg | — | | 287 | mg |
| Aerosil 200 | — | 10 | mg | — | | — | |
| Prosolv | — | — | | 287 | mg | — | |
| Croscarmellose sodium | — | — | | 22 | mg | — | |
| Plasdone K-29/32 | — | — | | — | | 22 | mg |
| Magnesium stearate | — | 5 | mg | 5 | mg | 5 | mg |

### Example 2

### Scale-Up of Solid Formulations from Example 1

Formulation 1 and Formulation 2 were scaled-up from 20 to 100 g (Tables 6-7; Formulation 1A and Formulation 2A, respectively). The granulation was done using a GMX top-drive high-shear granulation/mixing system where the Gelucire, Peceol and TPGS were dissolved in ethanol and this solution was added at 26 g/min and mixed to API at 60 rpm for 3 min. Powdered ingredients were separately mixed using a V-blender for 2 min. This powder blend was added to Gelucire/Peceol/TPGS/Ethanol/drug mixture and mixed for 6 min at impeller/chopper speeds 850/1800 rpm. The ethanol was then removed using a fluid bed dryer at 40° C. The fluidization was maintained until volatile compounds content was less or equal 3% (about 20 min). The volatile compounds content was determined by loss on drying (LOD) technique. The dried granules were sized by screening through an 18 mesh sieve followed by final lubrication.

TABLE 6
Formulation 1A

| Item | Ingredient | % w/w | mg/unit | g/batch |
|---|---|---|---|---|
| a | Amphotericin B | 23.0 | 100 | 23.0 |
| b | Mannitol 160C | 34.5 | 150 | 34.5 |
| c | Tabulose 101 | 34.3 | 149 | 34.3 |
| d | Colloidal silicon dioxide | 2.3 | 10 | 2.3 |
| e | TPGS | 0.2 | 1 | 0.2 |
| f | Peceol | 2.3 | 10 | 2.3 |
| g | Gelucire 44/14 | 2.3 | 10 | 2.3 |
| h | Ethanol 100% (evaporated | (30.0) | — | (30.0) |
| | during the process) | | | |
| i | Magnesium stearate | 1.1 | 5 | 1.1 |
| | Total: | 100 | 435 | 100 |

Items a-h are internal phase components, and item i is the external phase component.

TABLE 7
Formulation 2A

| Item | Ingredient | % w/w | mg/unit | g/batch |
|---|---|---|---|---|
| a | Amphotericin B | 23.0 | 100 | 23.0 |
| b | Prosolv HD90 | 66.0 | 287 | 66.0 |
| c | Croscarmellose sodium | 5.0 | 22 | 5.0 |
| d | TPGS | 0.2 | 1 | 0.2 |
| e | Peceol | 2.3 | 10 | 2.3 |
| f | Gelucire 44/14 | 2.3 | 10 | 2.3 |
| g | Ethanol 100% (evaporated | (30.0) | — | (30.0) |
| | during the process) | | | |
| h | Magnesium stearate | 1.1 | 5 | 1.1 |
| | Total: | 100 | 435 | 100 |

Items a-g are internal phase components, and item h is the external phase component.

### Example 3

### Semi-Solid Formulations

Semi-solid lipid based formulations (Formulation 4 and Formulation 5, Table 8A) filled into capsules for oral administration were prepared as per iCo formula composition (Table 8). However, in contrast to the iCo/Wasan liquid approach, a melt method was used. Indeed, the semi-solids excipients (Gelucire/TPGS) were melted, weighed and mixed with Peceol (liquid excipient) using a hot-plate magnetic-stirrer at 35-40° C. until a clear solution was obtained. The heating was stopped and the AmpB was added and mixed for 5 min. The liquid final blend was maintained under agitation and hot filled into size 00 hard gelatin capsules (fill weight: 804 mg) containing 4 mg AmpB. An additional lot was manufactured (Formulation 5) that contains the same amount of lipid excipients but more AmpB was ‘spiked’ to produce 100 mg dose capsules (fill weight: 900 mg).

TABLE 8A
iCo/Wasan Liquid Formulation, Formulation 4 and Formulation 5

| | iCo/Wasan Formulation | | | Formulation 4 | | Formulation 5 | |
|---|---|---|---|---|---|---|---|
| Ingredient | qty/dose | | % w/w | mg/dose | % w/w | mg/dose | % w/w |
| Amphotericin B | 150 | mg | 0.5 | 4 | 0.5 | 100 | 11.1 |
| TPGS | 1.5 | mL | 5 | 40 | 5 | 40 | 4.4 |
| Peceol | 14.25 | mL | 47.3 | 380 | 47.3 | 380 | 42.2 |
| Gelucire 44/14 | 14.25 | mL | 47.3 | 380 | 47.3 | 380 | 42.2 |
| Total: | 30 | mL | 100 | 804* | 100 | 900* | 100 |

*= 0.95 mL

### Example 4

### Scale Up of Semi-Solid Formulation from Example 3

The lipid based Formulation 5 was scaled-up from 23 to 360 g batch size (Table 8B). Each excipient was melted in its original container, followed by stirring to ensure homogeneity before sampling. The weighed molten samples were mixed together and AmpB was added under agitation. The mixture was maintained at 40° C. and under constant agitation for at least 30 minutes to ensure complete dispersion/solubilization. The final mixture was filled into hard gelatin capsules. Once the capsules' content cooldown to room temperature, the capsules were sealed using a mixture of purified water and Ethanol (50:50 v/v). A few droplets of solution were gently applied around the junction of the closed capsules' body and cap. Exceeding solution was immediately wiped out using a clean and dry cloth. The capsules were allowed to dry individually by resting vertically on a Cooper plate. Sealed capsules were stored at 4° C. until the start of the stability study.

TABLE 8B
100 mg Amphotericin B Lipid Formulation 5A

| Ingredient | mg/dose | % w/w | g/batch |
|---|---|---|---|
| Amphotericin B | 100 | 11.1 | 40.0 |
| TPGS | 40 | 4.4 | 16.0 |
| Peceol | 380 | 42.2 | 152.0 |
| Gelucire 44/14 | 380 | 42.2 | 152.0 |
| Total: | 900 | 100 | 360 |

### Example 5

### Physical and Chemical Characterization

Final blend was evaluated by bulk/tapped density and powder flow properties and residual solvents.

Bulk/Tapped Density-Powder Flow Properties-USP<616>

Bulk and tapped densities were determined using the USP <616>method with a Vanderkamp tap density tester model 10700 (VanKel Industries) and a Mettler Toledo balance model AT200. Each parameter was determined in duplicate using a 50 mL graduated glass cylinder. The bulk density was determined by measuring the volume of a known mass of powder sample in a graduated cylinder while the tapped density was measured by mechanically tapping the measuring cylinder until no further volume change was observed. The powder flow properties were evaluated using the Can's Compressibility Index and Hausner ratio as described in the next paragraphs.

Carr's Compressibility Index (CI): This flow property was calculated using bulk and tapped density data when fitted into the following equation:

Compressibility Index=(Tapped density−Bulk density)/Tapped density×100%

Hausner Ratio (H): This flow property was calculated as the ratio of tapped to bulk density.

The Compressibility Index (CI) and Hausner ratio (H) values interpretation as per USP <1174>as well as descriptive qualitative examples are presented in Table 9.

TABLE 9
Scale of Flowability

| Compressibility | Flow | Hausner | |
|---|---|---|---|
| Index (%) | Character | Ratio | Examples |
| ≤10 | Excellent | 1.00-1.11 | Free-flowing granules |
| 11-15 | Good | 1.12-1.18 | Powdered granules |
| 16-20 | Fair | 1.19-1.25 | Coarse powders |
| 21-25 | Passable | 1.26-1.34 | Fine powders |
| 26-31 | Poor | 1.35-1.45 | Fluidizable powders |
| 32-37 | Very poor | 1.46-1.59 | Cohesive powders |
| >38 | Very, very poor | >1.60 | Very cohesive powders |

Densities and powder flow properties are shown in Table 10. The formulations exhibit sufficient flowability. To fill 435 mg of final blend from Formulation 1 into size 0 capsules tapping and tamping was required. Bulk density could be increased by high shear granulation, using denser grades of excipients, e.g. microcrystalline cellulose type 200 or 302, or high functionality and multifunctional excipients such silicified microcrystalline cellulose (combination of microcrystalline cellulose and colloidal silicon dioxide). Silicified microcrystalline cellulose (Prosolv HD 90) has a bulk density 0.38-0.50 g/cm³ and was used in Formulation 2 resulting in an increased bulk density.

TABLE 10
Density and Flow Properties (n = 2)

| | | | Flow | | |
|---|---|---|---|---|---|
| | Density | | Properties | | |
| | Parameters | | Carr's | | |
| | Bulk | Tapped | Index | Hausner | Flow- |
| Lot No. | (g/cm³) | (g/cm³) | (%) | Ratio | ability |
| Formulation 1 | 0.40/0.41 | 0.49/0.50 | 18/18 | 1.22/1.22 | Fair |
| Formulation 2 | 0.46/0.45 | 0.54/0.53 | 15/14 | 1.18/1.17 | Good |
| Formulation 3 | 0.33/0.33 | 0.38/0.37 | 14/13 | 1.16/1.14 | Good |

### Capsule Weight Uniformity

As an example to show adequate flow properties, 12 capsules of Formulation 1 were tested for statistics (Table 11). Weight uniformity was confirmed with RSD <6.0% and no unit is outside the range of 85-115% of label claim.

TABLE 11
Encapsulation Statistics for 100 mg Amphotericin
B in Size 0 Capsules (n = 12) for Formulation 1.

| | Value |
|---|---|
| Statistic | (mg, Total weight) |
| Average | 527.8 |
| Stdev | 1.8 |
| RSD (%) | 0.3 |
| Min | 525.0 |
| Max | 529.9 |

The statistics for the semi-solid formulations are shown in Table 12. Formulation 4 capsules were filled by weight to approximately 90% of the capsule's volume to obtain 4 mg Amphotericin B/caps. Formulation 5 and Formulation 5A with 100 mg Amphotericin B/caps were filled to 100% of the capsule's volume.

TABLE 12
Encapsulation Statistics for Amphotericin B Semi-Solids
Formulations in Size 00 Capsules (n = 6) for
Formulation 4, Formulation 5, and Formulation 5A.

| | Formulation 4 | Formulation 5 | Formulation 5A |
|---|---|---|---|
| | (4 mg Amp. B/ | (100 mg Amp. B/ | (100 mg Amp. B/ |
| Statistics | cap) | caps) | caps) |
| Average Total | 919.1 | 1014.8 | 1011.9 |
| Wt (mg) | | | |
| Stdev | 0.9 | 6.9 | 7.5 |
| RSD (%) | 0.1 | 0.7 | 0.7 |
| Min | 918.1 | 1003.5 | 1003.8 |
| Max | 920.4 | 1022.1 | 1027.1 |

### Residual Solvents

Determination of residual solvents was carried out by thermal gravimetric analysis (TGA) using a TA Instrument Q50 thermogravimetric analyzer at scanning speed of 10° C. min⁻¹ over a temperature range of 25 to 200° C. The samples (11-13mg) were heated in a platinum open pan in nitrogen atmosphere (60 mL min⁻¹).

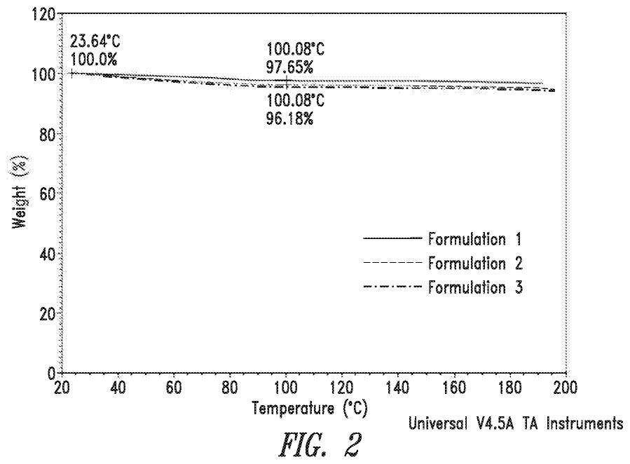

TGA curves of Amphotericin B (23%) solid oral dose formulations' final blends are illustrated in FIG. 2. TGA show weight loss of 2.4-3.8% between 25 and 100° C. which is typically associated with evaporation of volatiles compounds (solvents and moisture). This weight loss is low considering that the moisture content of microcrystalline cellulose is between 3 and 5% (moisture data from CofA). As a consequence, for samples containing higher quantity of microcrystalline cellulose such as Formulation 2 and Formulation 3, it is normal that the weight loss appeared slightly increased (3.8%) when compared with Formulation 1 (2.4%).

### Example 6

### Analytical Testing

### Formulation 1

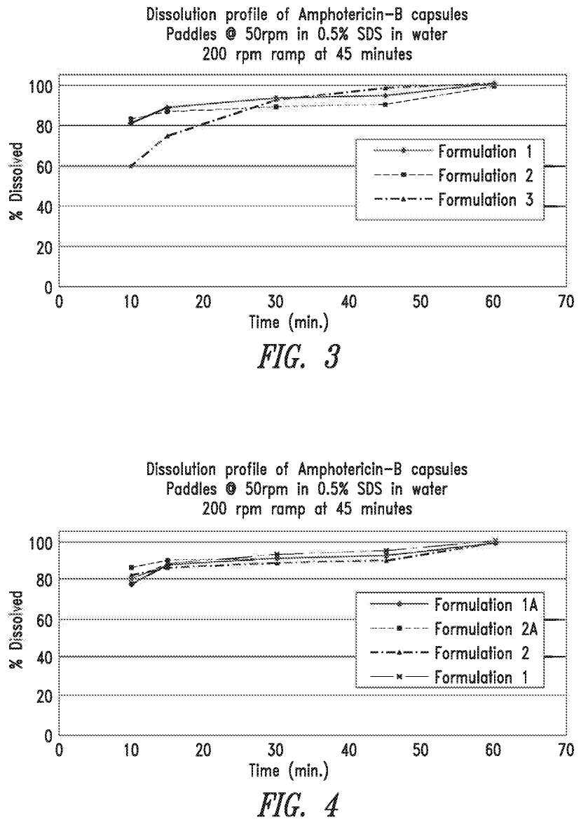

The assay and related substances results for Amphotericin B formulations are shown in Table 13. Replicate 1 and 2 were prepared using Sample Preparation 1 and Replicate 3 was prepared using Sample Preparation 2, in an attempt to minimise the consumption of NMP in the diluent. Similar extraction efficiency was obtained with both sample preparation procedures. The extraction procedure for the assay and related substances achieved ˜95% recovery. The dissolution profile is fairly rapid with 95% released at 45 minutes (FIG. 3). After an increase in paddle speed, 100% released was achieved.

TABLE 13
Analytical Results for 100 mg Amphotericin B capsules Formulation 1.

| | Formulation 1 | | | | |
|---|---|---|---|---|---|
| Sample | 100 mg/capsule | | | | |
| Dose strength | API/Gelucire/Peceol/TPGS/ | | | | |
| Formulation | Mannitol/MCC/CSD/MgSt | | | | |
| Appearance | Yellow powder in white capsule | | | | |
| | Sample | | | Sample | |
| | Preparation 1 | | | Preparation 2 | |
| Assay | 93.4 | | | 95.9 | |
| % of nominal content | (n = 2: 93.2, 93.7) | | | (n = 1) | |
| Corealis -26801-AD-01 | | | | | |
| Rev R&D 02 | | | | | |
| | | % area | % area | | |
| | | Replicate | Replicate | | |
| | RRT | 1 | 2 | RRT | % area |
| Related Substances | 0.29 | 0.85 | 0.87 | 0.29 | 1.05 |
| Corealis -26801-AD-01 | 0.48 | ND | ND | 0.48 | 0.12 |
| Rev R&D 02 | 0.63 | 0.65 | 0.68 | 0.63 | 0.70 |
| | 0.69 | 0.11 | 0.11 | 0.69 | 0.12 |
| | 0.73 | 0.32 | 0.33 | 0.73 | 0.33 |
| | 0.76 | 0.35 | 0.35 | 0.76 | 0.35 |
| | 0.79 | 0.25 | 0.26 | 0.79 | 0.26 |
| | 0.82 | 0.65 | 0.65 | 0.82 | 0.66 |
| | 0.91 | 0.12 | 0.12 | 0.91 | 0.13 |
| | 1.25 | 0.87 | 0.79 | 1.25 | 0.72 |
| | 1.29 | 0.54 | 0.55 | 1.29 | 0.55 |
| | 1.36 | 0.21 | 0.18 | 1.36 | 0.16 |
| | 1.39 | 1.14 | 0.67 | 1.39 | 0.29 |
| | 1.47 | 0.41 | 0.23 | 1.47 | ND |
| | 1.68 | 0.38 | 0.15 | 1.68 | ND |
| | 1.79 | 1.06 | 1.06 | 1.79 | 1.05 |
| | 1.91 | 0.12 | 0.13 | 1.91 | 0.13 |
| | 2.32 | 1.45 | 1.44 | 2.32 | ND |
| | 2.38 | ND | ND | 2.38 | 1.34 |
| | Total | 9.6 | 8.6 | Total | 8.0 |

| | Peaks ≥0.10% reported | Peaks ≥0.10% reported |
|---|---|---|
| | | % dissolved |
| | Time (min.) | (range) |
| Dissolution | 10 | 81 (76-85) |
| 900 ml 0.5% SDS in Water | 15 | 89 (87-90) |
| paddles at 50 rpm | 30 | 93 (85-93) |
| % dissolved | 45 | 95 (95-95) |
| Corealis -26801-B-01 | 60 | 101 (100-102) |
| Rev R&D 0 | (ramp to 200 rpm | |
| (n = 3) | after 45 min) | |

### Formulation 2 and Formulation 3

The assay and related substances results for Amphotericin B carried out using a modified extraction procedure are shown in Table 14. The contents and emptied capsule shells from 4 capsules were extracted per sample replicate using Sample Preparation 3 (see Method for details). Some samples required much longer sonication time to break up the agglomerates formed. The longer sonication time also increased the amount of degradation produced during sample preparation. The dissolution profiles are shown in FIG. 3. Formulation 3 appears somewhat slower initially but rapidly rejoins the profiles of the other formulations.

Table 15 shows the Impurity profile of AmpB used in the formulations which indicates that the process used to produce the dosage forms did not affect adversely the AmpB.

TABLE 14
Analytical Results for 100 mg Amphotericin
B capsules Formulation 2 and Formulation 3.

| | Formulation 2 | | | Formulation 3 | | |
|---|---|---|---|---|---|---|
| Sample | 100 mg/capsule | | | | | |
| Dose strength | API/Gelucire/Peceol/TPGS/ | | | API/Gelucire/Peceol/TPGS/ | | |
| Formulation | SMCC/CC/MgSt | | | MCC/PVP/MgSt | | |
| Appearance | Yellow powder in white capsule | | | | | |
| Assay % LC | 83.1% ^{A} | | | 89.0 | | |
| Corealis -26801-AD-01 | (80.3, 85.9) | | | (91.1, 86.8) | | |
| Rev R&D 03 | | | | | | |
| | RRT | % area | | RRT | % area | |
| Related Substances | 0.19 | 0.10 | | 0.19 | 0.10 | |
| Corealis -26801-AD-01 | 0.31 | 1.05 | | 0.31 | 1.10 | |
| Rev R&D 03 | 0.50 | 0.22 | | 0.49 | 0.14 | |
| (% a/a) | 0.63 | 0.62 | | 0.63 | 0.67 | |
| | 0.69 | 0.13 | | 0.69 | 0.13 | |
| | 0.73 | 0.33 | | 0.73 | 0.35 | |
| | 0.74 | 0.31 | | 0.74 | 0.32 | |
| | 0.78 | 0.22 | | 0.78 | 0.25 | |
| | 0.83 | 0.58 | | 0.82 | 0.60 | |
| | 0.88 | 0.20 | | 0.88 | 0.11 | |
| | 1.12 | 0.14 | | 1.22 | 0.84 | |
| | 1.23 | 1.06 | | 1.28 | 0.71 | |
| | 1.28 | 0.71 | | 1.33 | 0.28 | |
| | 1.33 | 0.34 | | 1.36 | 1.16 | |
| | 1.36 | 2.24 | | 1.45 | 0.47 | |
| | 1.45 | 0.74 | | 1.58 | 0.11 | |
| | 1.58 | 0.13 | | 1.66 | 0.60 | |
| | 1.66 | 1.81 | | 1.83 | 0.99 | |
| | 1.83 | 0.99 | | 1.96 | 0.21 | |
| | 1.96 | 0.26 | | 2.36 | 1.34 | |
| | 1.99 | 0.13 | | Total | 10.5 | |
| | 2.36 | 1.38 | | | | |
| | Total | 13.7 | | | | |
| | Time | % dissolved | | Time | % dissolved | |
| | (min.) | (range) | | (min.) | (range) | |
| Dissolution | 10 | 83 | (77-91) | 10 | 60 | (51-76) |
| 900 ml 0.5% SDS in Water | 15 | 87 | (80-95) | 15 | 75 | (59-89) |
| paddles at 50 rpm | 30 | 89 | (83-96) | 30 | 93 | (90-98) |
| % dissolved | 45 | 90 | (83-96) | 45 | 99 | (97-101) |
| Corealis -26801-B-01 | 60 | 100 | (99-101) | 60 | 101 | (99-103) |
| Rev R&D 0 | (ramp) | | | (ramp) | | |
| (n = 3) | | | | | | |

Peaks ≥0.10% reported
^{A} One replicate was sonicated significantly longer to break up agglomerates present in sample which resulted in higher levels of degradation.

TABLE 9
Impurity Profile of AMpB

| | |
|---|---|
| Formuation 1 | Formulations 2 and 3 |

| RRT | % area | RRT | % area |
|---|---|---|---|
| 0.29 | 0.88 | 0.30 | 0.94 |
| 0.48 | 0.11 | 0.49 | 0.11 |
| 0.62 | 0.63 | 0.62 | 0.66 |
| 0.69 | 0.12 | 0.69 | 0.13 |
| 0.73 | 0.33 | 0.73 | 0.61 |
| 0.75 | 0.34 | 0.75 | ND |
| 0.78 | 0.26 | 0.78 | 0.20 |
| 0.82 | 0.65 | 0.82 | 0.60 |
| 0.88 | ND | 0.88 | 0.29 |
| 0.91 | 0.12 | 0.91 | ND |
| 1.08 | ND | 1.08 | 0.10 |
| 1.22 | ND | 1.22 | 0.66 |
| 1.25 | 0.72 | 1.25 | ND |
| 1.27 | ND | 1.27 | 0.73 |
| 1.29 | 0.60 | 1.29 | ND |
| 1.35 | 0.11 | 1.32 | 0.13 |
| 1.39 | 0.15 | 1.39 | ND |
| 1.79 | 1.06 | 1.79 | ND |
| 1.82 | ND | 1.82 | 1.05 |
| 1.91 | 0.14 | 1.96 | 0.13 |
| 2.32 | 1.43 | 2.35 | 1.38 |
| Total | 7.7 | Total | 7.7 |

Peaks ≥0.10% reported

### Formulation 1A and Formulation 2A

The assay and related substances for Amphotericin-B in capsule was carried out at the initial time point (T=0) using a modified extraction procedure (Table 16). The contents from 3 capsules were extracted per sample replicate using Sample Preparation 4 (see Methods for details). The dissolution profiles for the scale-up lots are comparable to the previous lots (FIG. 4).

TABLE 16
Analytical Results for 100 mg Amphotericin B
capsules of Formulation 1A and Formulation 2A

| Sample | Formulation 1A | | | Formulation 2A | | |
|---|---|---|---|---|---|---|
| Formulation | Amphotericin B with Gelucire | | | Amphotericin B with Gelucire 44/14- | | |
| | 44/14-Peceol-TPGS (10 mg-10 mg-1 mg), | | | Peceol-TPGS (10 mg-10 mg-1 mg), | | |
| | mannitol/microcrystalline | | | silicified microcrystalline | | |
| | cellulose oral dose formulation in | | | cellulose/croscarmellose oral dose | | |
| | hard shell capsule | | | formulation in hard shell capsule | | |
| | Scale-up Formulation 1 | | | Scale-up Formulation 2 | | |
| Dose strength | 100 mg/capsule | | | | | |
| Appearance | Yellow powder in white capsule (slight agglomerations) | | | | | |
| Water content | 2.9% | | | 3.8% | | |
| Assay | Sample Preparation 4 | | | | | |
| % LC | 98.6% | | | 101.5 | | |
| Corealis - | (100.6, 96.5) | | | (101.5, 101.6) | | |
| 26801-AD-01 | | | | | | |
| Rev R&D 04 | | | | | | |
| | RRT | % area | | RRT | % area | |
| Related | 0.30 | 0.79 | | 0.18 | 0.11 | |
| Substances | 0.49 | 0.13 | | 0.30 | 0.79 | |
| Corealis - | 0.62 | 0.54 | | 0.49 | 0.13 | |
| 26801-AD-01 | 0.69 | 0.13 | | 0.62 | 0.38 | |
| Rev R&D 04 | 0.73 | 0.32 | | 0.69 | 0.14 | |
| (% a/a) | 0.75 | 0.33 | | 0.73 | 0.34 | |
| | 0.79 | 0.23 | | 0.75 | 0.32 | |
| | 0.82 | 0.59 | | 0.79 | 0.22 | |
| | 0.85 | 0.31 | | 0.82 | 0.59 | |
| | 0.90 | 0.11 | | 0.85 | 0.31 | |
| | 1.17 | 0.64 | | 0.90 | 0.11 | |
| | 1.21 | 0.61 | | 1.17 | 0.61 | |
| | 1.27 | 0.32 | | 1.21 | 0.58 | |
| | 1.78 | 1.00 | | 1.27 | 0.30 | |
| | 2.16 | 1.42 | | 1.78 | 1.00 | |
| | Total | 7.47 | | 2.16 | 1.40 | |
| | | | | Total | 7.35 | |
| | Time (min.) | % dissolved | Range | Time (min.) | % dissolved | Range |
| Dissolution | 10 | 78 | (69-84) | 10 | 86 | (81-95) |
| 900 ml 0.5% | 15 | 88 | (77-96) | 15 | 90 | (86-97) |
| | 30 | 91 | (82-100) | 30 | 91 | (88-97) |
| Formulation | Amphotericin B with Gelucire | | | Amphotericin B with Gelucire 44/14- | | |
| | 44/14-Peceol-TPGS (10 mg-10 mg-1 mg), | | | Peceol-TPGS (10 mg-10 mg-1 mg), | | |
| | mannitol/microcrystalline | | | silicified microcrystalline | | |
| | cellulose oral dose formulation in | | | cellulose/croscarmellose oral dose | | |
| | hard shell capsule | | | formulation in hard shell capsule | | |
| | Scale-up Formulation 1 | | | Scale-up Formulation 2 | | |
| Dose strength | 100 mg/capsule | | | | | |
| SDS in Water | 45 | 92 | (83-100) | 45 | 92 | (88-98) |
| paddles at 50 rpm | 60 (ramp) | 99 | (93-102) | 60 (ramp) | 99 | (98-99) |
| % dissolved | | | | | | |
| Corealis - | | | | | | |
| 26801-B-01 | | | | | | |
| Rev R&D 02 | | | | | | |
| (n = 3) | | | | | | |

Peaks ≥ 0.10% reported

### Formulation 4 and Formulation 5

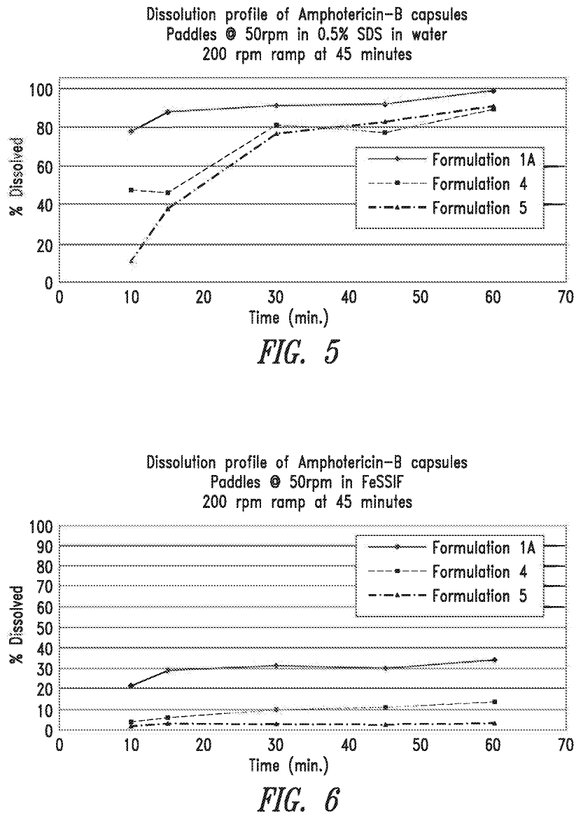

Semi-solid lipid based formulations in capsules for oral administration were prepared as per iCo formula composition and Corealis modified process. The capsules were analysed for dissolution profiles in the current 0.1N HCl+0.5% SDS medium and in Simulated Fed Intestinal Fluid (FeSSIF pH 5.8) (Table 17, FIG. 5).

The semi-solid formulations Formulation 4 (0.5% Drug Load) and Formulation 5 (11.1% Drug Load) showed slightly slower dissolution profiles in 0.5% SDS in water up to 30 minutes when compared the ‘solid’ capsule Formulation 1A (23% Drug Load). In the FeSSIF pH 5.8 medium where Amphotericin B may be solubility limited, the dissolution profiles reached a maximum of ˜35% dissolved for the ‘solid’ capsule formulation and less than 15% dissolved for the semi-solid formulations (FIG. 6). In this in vitro model, the semi-solid formulations with the increased lipid concentration do not show improved dissolution profile. Both, Formulation 4 and Formulation 5 showed similar end results when compared to the solid oral dosage form Formulation 1A.

TABLE 17
Dissolution Results for Amphotericin B Semi-solid Capsules Formulation 1A, Formulation 4, and Formulation 5.

| Sample | Formulation 4 | | | Formulation 5 | | | Formulation 1A | | |
|---|---|---|---|---|---|---|---|---|---|
| Formulation | Semi-solid 0.5% Drug Load - | | | Semi-solid 11.1% Drug Load - | | | Solid Oral 23% Drug Load - | | |
| | API/Gelucire/Peceol/TPGS in | | | API/Gelucire/Peceol/TPGS in | | | API/Gelucire/Peceol/TPGS/ | | |
| | hard shell capsule | | | hard shell capsule | | | mannitol/microcrystalline cellulose | | |
| | | | | | | | in hard shell capsule | | |
| Dose strength | 4 mg/caps | | | 100 mg/caps | | | | | |
| | 500 ml 0.5% SDS in water | | | 900 ml 0.5% SDS in water | | | 900 ml 0.5% SDS in water (n = 3) | | |
| | Time (min.) | % dissolved | Range | Time (min.) | % dissolved | Range | Time (min.) | % dissolved | Range |
| Dissolution | 10 | 47 | (38-55) | 10 | 11 | (6-15) | 10 | 78 | (69-84) |
| paddles at | 15 | 46^{A} | (46) | 15 | 38 | (31-45) | 15 | 88 | (77-96) |
| 50 rpm | 30 | 81 | (70-91) | 30 | 77 | (71-84) | 30 | 91 | (82-100) |
| % dissolved | 45 | 77 | (66-88) | 45 | 83 | (77-89) | 45 | 92 | (83-100) |
| Corealis - | 60 (ramp) | 89 | (87-91) | 60 (ramp) | 91 | (89-94) | 60 (ramp) | 99 | (93-102) |
| 26801-B-01 | | | | | | | | | |
| Rev R&D 02 | | | | | | | | | |
| (n = 2) | | | | | | | | | |
| | 500 ml FeSSIF-V2 pH 5.8 ^{B} | | | 900 ml FeSSIF-V2 pH 5.8 ^{B} | | | 900 ml FeSSIF-V2 pH 5.8 ^{B} | | |
| | Time (min.) | % dissolved | Range | Time (min.) | % dissolved | Range | Time (min.) | % dissolved | Range |
| | 10 | 4 | (3-5) | 10 | 2 | (2-2) | 10 | 21 | (20-23) |
| | 15 | 6 | (5-8) | 15 | 3 | (2-4) | 15 | 29 | (28-31) |
| | 30 | 10 | (7-13) | 30 | 3 | (3-3) | 30 | 31 | (30-32) |
| | 45 | 11 | (9-13) | 45 | 3 | (3-4) | 45 | 30 | (29-30) |
| | 60 (ramp) | 14 | (14-15) | 60 (ramp) | 4 | (3-4) | 60 (ramp) | 34 | (33-36) |

^{A}One injection of sample L268-01021 at 15 minutes showed 130% dissolved, at subsequent time points amount ~90% dissolved is observed. The vial was reinjected and the peak area did not change. This atypical value is not reported.

### Formulation 5 and Formulation 5A

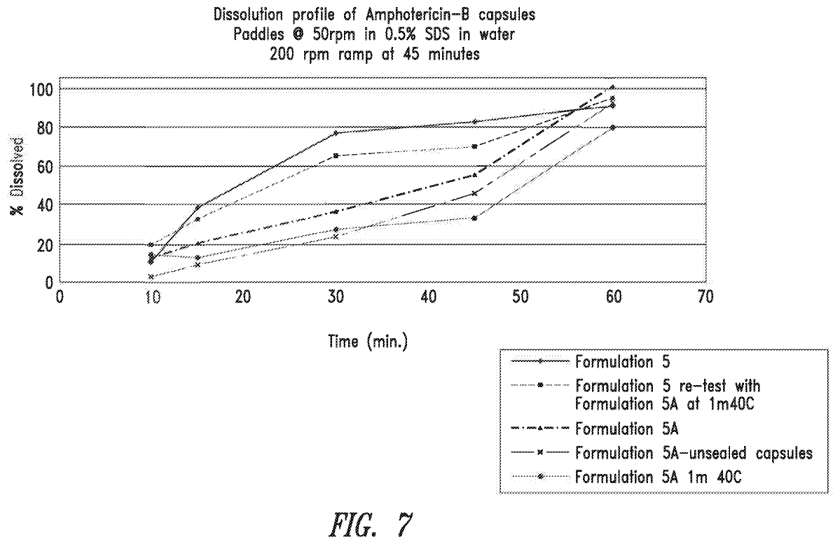

Formulation 5 and Formulation 5A are the same composition but prepared at different scale. The mixing time was increased consequently. Moreover, Formulation 5A and Formulation 5A-1capsules comes from the same final blend with only one difference whereby Formulation 5A capsules were sealed and Formulation 5A-1 were filled later and not sealed. The initial/T=0 data shown in FIG. 7, revealed that the dissolution profiles were different for all three lots. However, after 60 minutes 90-100% of AmpB was dissolved. Subsequently, it was also discovered that lower dissolution profiles were observed for Formulation 5A stored for 1 month at 40° C./75% RH as well as for Formulation 5 stored at 5° C. for about 5 months.

Without being bound by theory, the decrease of dissolution profile as a function of time could be ascribed to different degrees of solubilizing of the AmpB during the processing of the different batch size lots.

### Example 7

### Stability Study

### Formulation 1A and Formulation 2A

A stability study was initiated for Formulation 1A and Formulation 2A. The capsules were packaged in 30 cc HDPE bottles with induction sealed PP caps and the bottles were stored under ICH stability conditions in humidity chambers at 25° C./60% RH and under accelerated conditions, 40° C./75% RH. The capsules were stored at 4-8° C. directly after preparation until they were placed into the stability chambers.

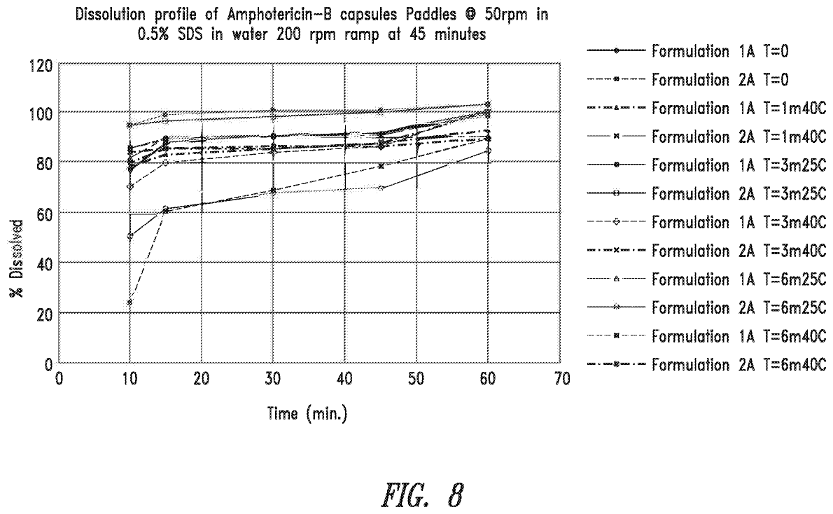

Stability testing results for 100 mg Amphotericin B capsules Formulation 1A and Formulation 2A are summarized in Tables 18 to 20. Dissolution profiles were compared in FIG. 8. Both formulations are stable for up to 6 months at 25° C./60% RH and 40° C./75% RH with no significant changes in assay, related substances, and dissolution profile when compared to initial (T=0) results.

TABLE 18
Stability Testing Results for Formulation 1A and Formulation 2A

| Sample | | Formulation 1A | Formulation 2A |
|---|---|---|---|
| Dose strength | | 100 mg/capsule | |
| Appearance | T = 0 | Yellow powder in white capsule | |
| | | (slight agglomerations) | |
| | T = 1 month | Yellow powder in white capsule | |
| | 40° C./75% RH | (slight agglomerations) | |
| | T = 3 months | Yellow powder in white capsule | |
| | 25° C./60% RH | (slight agglomerations) | |
| | T = 3 months | Yellow powder in white capsule | |
| | 40° C./75% RH | (slight agglomerations) | |
| | T = 6 months | Yellow powder in white capsule | |
| | 25° C./60% RH | (slight agglomerations) | |
| | T = 6 months | Yellow powder in white capsule | |
| | 40° C./75% RH | (slight agglomerations) | |
| Water content | T = 0 | 2.9% | 3.8% |
| | T = 1 month | 3.2% | 4.1% |
| | 40° C./75% RH | | |
| | T = 3 months | 3.1% | 4.0% |
| | 25° C./60% RH | | |
| | T = 3 months | 3.2% | 4.4% |
| | 40° C./75% RH | | |
| | T = 6 months | 2.8% | 3.6% |
| | 25° C./60% RH | | |
| | T = 6 months | 3.1% | 4.1% |
| | 40° C./75% RH | | |
| Assay (% LC) | T = 0 | 98.6% | 101.5% |
| Corealis -26801-AD-01 | | (n = 2: 100.6, 96.5) | (n = 2: 101.5, 101.6) |
| Rev R&D 04 | T = 1 month | 98.9% | 101.6% |
| (T = 0 to T = 3 m) | 40° C./75% RH | | |
| Corealis -26801-AD-01 | | (n = 2: 100.4, 97.3) | (n = 2: 102.8, 100.4) |
| Rev R&D 09 | T = 3 months | 100.0% | 97.8% |
| (T = 6 m) | 25° C./60% RH | | |
| | | (n = 2: 100.2, 99.7) | (n = 2: 99.1, 96.5) |
| | T = 3 months | 97.2% | 99.3% |
| | 40° C./75% RH | | |
| | | (n = 2: 95.7, 98.6) | (n = 2: 100.5, 98.0) |
| | T = 6 months | 96.9% | 93.0% |
| | 25° C./60% RH | | |
| | | (n = 5: 102.0, 96.3, | (n = 5: 94.7, 95.3, |
| | | 100.8, 90.3, 95.2) | 95.2, 89.4, 90.6) |
| | T = 6 months | 93.5% | 91.8% |
| | 40° C./75% RH | | |
| | | (n = 5: 96.5, 90.0, | (n = 5: 89.3, 89.6, |
| | | 94.4, 92.7, 94.2) | 93.0, 97.1, 89.8) |

Note:
The assay is quantitated against the API only. Chromatographic impurities are not taken into account.

TABLE 19
Related Substances for Formulation 1A and Formulation 2A vs AmpB

| Sample | | Formulation 1A | | Formulation 2A | | AmpB (Corealis lot C00729) | |
|---|---|---|---|---|---|---|---|
| | | RRT | % area | RRT | % area | RRT | % area |
| Related | T = 0 | 0.30 | 0.79 | 0.18 | 0.11 | 0.29 | 0.82 |
| Substances | | 0.49 | 0.13 | 0.30 | 0.79 | 0.48 | 0.14 |
| (% a/a) | | 0.62 | 0.54 | 0.49 | 0.13 | 0.62 | 0.54 |
| Corealis - | | 0.69 | 0.13 | 0.62 | 0.38 | 0.68 | 0.12 |
| 26801-AD-01 | | 0.73 | 0.32 | 0.69 | 0.14 | 0.73 | 0.32 |
| Rev R&D 04 | | 0.75 | 0.33 | 0.73 | 0.34 | 0.74 | 0.33 |
| (T = 0 to | | 0.79 | 0.23 | 0.75 | 0.32 | 0.78 | 0.24 |
| T = 3 m) | | 0.82 | 0.59 | 0.79 | 0.22 | 0.82 | 0.60 |
| Corealis - | | 0.85 | 0.31 | 0.82 | 0.59 | 0.85 | 0.31 |
| 26801-AD-01 | | 0.90 | 0.11 | 0.85 | 0.31 | 0.89 | 0.12 |
| Rev R&D 09 | | 1.17 | 0.64 | 0.90 | 0.11 | 1.17 | 0.65 |
| (T = 6 m) | | 1.21 | 0.61 | 1.17 | 0.61 | 1.21 | 0.69 |
| | | 1.27 | 0.32 | 1.21 | 0.58 | 1.27 | 0.30 |
| | | 1.78 | 1.00 | 1.27 | 0.30 | 1.78 | 1.00 |
| | | 2.16 | 1.42 | 1.78 | 1.00 | 2.17 | 1.47 |
| | | Total | 7.47 | 2.16 | 1.40 | Total | 7.67 |
| | | | | Total | 7.35 | | |
| | T = 1 month | 0.29 | 0.78 | 0.17 | 0.12 | 0.29 | 0.79 |
| | 40° C./75% RH | 0.48 | 0.13 | 0.29 | 0.78 | 0.48 | 0.13 |
| | | 0.62 | 0.39 | 0.48 | 0.13 | 0.62 | 0.57 |
| | | 0.69 | 0.15 | 0.62 | 0.31 | 0.69 | 0.16 |
| | | 0.73 | 0.31 | 0.69 | 0.15 | 0.73 | 0.32 |
| | | 0.75 | 0.28 | 0.73 | 0.31 | 0.75 | 0.33 |
| | | 0.79 | 0.18 | 0.75 | 0.27 | 0.79 | 0.24 |
| | | 0.82 | 0.57 | 0.79 | 0.20 | 0.82 | 0.59 |
| | | 0.85 | 0.35 | 0.82 | 0.57 | 0.85 | 0.31 |
| | | 0.90 | <0.10 | 0.85 | 0.33 | 0.90 | 0.11 |
| | | 1.17 | 0.62 | 0.90 | <0.10 | 1.17 | 0.62 |
| | | 1.22 | 0.69 | 1.17 | 0.62 | 1.22 | 0.69 |
| | | 1.27 | 0.37 | 1.22 | 0.67 | 1.27 | 0.31 |
| | | 1.78 | 1.00 | 1.27 | 0.37 | 1.77 | 1.00 |
| | | 2.16 | 1.30 | 1.77 | 1.00 | 2.16 | 1.52 |
| | | Total | 7.13 | 2.16 | 1.25 | Total | 7.71 |
| | | | | 3.05 | 0.11 | | |
| | | | | 3.68 | 0.12 | | |
| | | | | Total | 7.31 | | |
| | T = 3 months | 0.13* | 0.15 | 0.13* | 0.17 | 0.13* | 0.14 |
| | 25° C./60% RH | 0.30 | 0.79 | 0.18 | 0.12 | 0.30 | 0.78 |
| | | 0.51 | 0.13 | 0.30 | 0.80 | 0.51 | 0.15 |
| | | 0.64 | 0.42 | 0.51 | 0.13 | 0.64 | 0.48 |
| | | 0.71 | 0.16 | 0.64 | 0.25 | 0.70 | 0.15 |
| | | 0.75 | 0.36 | 0.71 | 0.16 | 0.75 | 0.35 |
| | | 0.76 | 0.21 | 0.75 | 0.36 | 0.76 | 0.22 |
| | | 0.80 | 0.14 | 0.76 | 0.20 | 0.80 | 0.15 |
| | | 0.83 | 0.53 | 0.80 | 0.14 | 0.83 | 0.54 |
| | | 0.85 | 0.29 | 0.83 | 0.52 | 0.85 | 0.25 |
| | | 1.14 | 0.56 | 0.85 | 0.28 | 1.14 | 0.57 |
| | | 1.18 | 0.66 | 1.14 | 0.56 | 1.18 | 0.77 |
| | | 1.23 | 0.18 | 1.19 | 0.64 | 1.23 | 0.13 |
| | | 1.25 | 0.18 | 1.23 | 0.18 | 1.25 | 0.16 |
| | | 1.73 | 1.04 | 1.25 | 0.20 | 1.73 | 1.03 |
| | | 2.07 | 1.27 | 1.73 | 1.04 | 2.06 | 1.37 |
| | | 2.10 | 0.13 | 2.07 | 1.21 | 2.10 | 0.14 |
| | | Total | 7.21 | 2.10 | 0.13 | Total | 7.38 |
| | | | | Total | 7.08 | | |
| | T = 3 months | 0.13* | 0.18 | 0.13* | 0.20 | | |
| | 40° C./75% RH | 0.18 | 0.10 | 0.18 | 0.14 | | |
| | | 0.30 | 0.78 | 0.30 | 0.80 | | |
| | | 0.51 | 0.14 | 0.51 | 0.13 | | |
| | | 0.64 | 0.26 | 0.64 | 0.17 | | |
| | | 0.71 | 0.17 | 0.71 | 0.17 | | |
| | | 0.75 | 0.56 | 0.75 | 0.54 | | |
| | | 0.80 | 0.18 | 0.80 | 0.17 | | |
| | | 0.83 | 0.53 | 0.83 | 0.52 | | |
| | | 0.85 | 0.33 | 0.85 | 0.32 | | |
| | | 1.14 | 0.56 | 1.14 | 0.55 | | |
| | | 1.19 | 0.83 | 1.19 | 0.78 | | |
| | | 1.23 | 0.19 | 1.23 | 0.18 | | |
| | | 1.25 | 0.13 | 1.25 | 0.11 | | |
| | | 1.73 | 1.04 | 1.73 | 1.03 | | |
| | | 2.07 | 1.03 | 2.07 | 0.97 | | |
| | | 2.10 | 0.13 | 2.10 | 0.12 | | |
| | | 3.54 | 0.10 | 2.92 | 0.18 | | |
| | | Total | 7.24 | 3.54 | 0.16 | | |
| | | | | Total | 7.23 | | |
| | T = 6 months | 0.14* | 0.19 | 0.14* | 0.24 | 0.14* | 0.12 |
| | 25° C./60% RH | 0.30 | 0.85 | 0.19 | 0.14 | 0.30 | 0.86 |
| | | 0.49 | 0.13 | 0.30 | 0.83 | 0.49 | 0.14 |
| | | 0.62 | 0.37 | 0.49 | 0.13 | 0.62 | 0.52 |
| | | 0.69 | 0.13 | 0.62 | 0.21 | 0.68 | 0.14 |
| | | 0.73 | 0.61 | 0.69 | 0.13 | 0.73 | 0.64 |
| | | 0.78 | 0.18 | 0.73 | 0.60 | 0.78 | 0.20 |
| | | 0.83 | 0.79 | 0.78 | 0.17 | 0.83 | 0.71 |
| | | 1.14 | 0.55 | 0.83 | 0.76 | 1.14 | 0.56 |
| | | 1.18 | 0.67 | 1.14 | 0.55 | 1.18 | 0.71 |
| | | 1.22 | 0.18 | 1.18 | 0.63 | 1.22 | 0.10 |
| | | 1.26 | 0.14 | 1.23 | 0.17 | 1.26 | 0.15 |
| | | 1.77 | 1.01 | 1.26 | 0.12 | 1.77 | 1.01 |
| | | 2.10 | 1.15 | 1.78 | 1.00 | 2.10 | 1.32 |
| | | 2.16 | 0.11 | 2.10 | 1.06 | Total | 7.18 |
| | | Total | 7.07 | 2.16 | 0.11 | | |
| | | | | 4.01 | 0.11 | | |
| | | | | Total | 6.96 | | |
| | T = 6 months | | | 0.14* | 0.23 | 0.14* | 0.31 |
| | 40° C./75% RH | | | 0.19 | 0.12 | 0.19 | 0.18 |
| | | | | 0.30 | 0.84 | 0.30 | 0.81 |
| | | | | 0.49 | 0.14 | 0.49 | 0.14 |
| | | | | 0.62 | 0.14 | 0.69 | 0.15 |
| | | | | 0.68 | 0.15 | 0.73 | 0.50 |
| | | | | 0.73 | 0.56 | 0.78 | 0.18 |
| | | | | 0.78 | 0.17 | 0.83 | 0.86 |
| | | | | 0.83 | 0.85 | 1.14 | 0.53 |
| | | | | 1.14 | 0.55 | 1.18 | 0.90 |
| | | | | 1.18 | 0.97 | 1.23 | 0.15 |
| | | | | 1.22 | 0.17 | 1.64 | 0.69 |
| | | | | 1.26 | 0.10 | 1.77 | 0.99 |
| | | | | 1.77 | 1.00 | 2.10 | 0.80 |
| | | | | 2.10 | 0.88 | 2.16 | 0.12 |
| | | | | Total | 6.88 | Total | 7.32 |

Note:
Peaks ≥ 0.10% reported; the reported impurity profile (% area) is equivalent in the API and sample solutions.
*The peak was not integrated at previous time point since it was broader, now sharper peak is observed and reported.

TABLE 20
Stability Testing Results (Dissolution) for Formulation 1A and Formulation 2A

| Sample | | Formulation 1A | | | Formulation 2A | | |
|---|---|---|---|---|---|---|---|
| Formulation | | Amphotericin B with Gelucire | | | Amphotericin B with Gelucire | | |
| | | 44/14-Peceol-TPGS (10 mg-10 mg-1 mg), | | | 44/14-Peceol-TPGS (10 mg-10 mg-1 mg), | | |
| | | mannitol/microcrystalline | | | silicified microcrystalline | | |
| | | cellulose oral dose formulation in | | | cellulose/croscarmellose oral | | |
| | | hard shell capsule | | | dose formulation in hard shell | | |
| | | | | | capsule | | |
| Dose strength | | 100 mg/capsule | | | | | |
| | | Time (min.) | % dissolved | Range | Time (min.) | % dissolved | Range |
| Dissolution | T = 0 | 10 | 78 | (69-84) | 10 | 86 | (81-95) |
| 900 ml 0.5% SDS | | 15 | 88 | (77-96) | 15 | 90 | (86-97) |
| in Water | | 30 | 91 | (82-100) | 30 | 91 | (88-97) |
| paddles at 50 | | 45 | 92 | (83-100) | 45 | 92 | (88-98) |
| rpm | | 60 (ramp) | 99 | (93-102) | 60 (ramp) | 99 | (98-99) |
| % dissolved | T = 1 month | 10 | 80 | (74-84) | 10 | 86 | (80-95) |
| Corealis -26801- | 40° C./75% RH | 15 | 84 | (80-88) | 15 | 89 | (83-98) |
| B-01 Rev R&D 02 | | 30 | 86 | (83-90) | 30 | 91 | (85-100) |
| (n = 3) | | 45 | 88 | (85-91) | 45 | 91 | (86-101) |
| | | 60 (ramp) | 93 | (89-95) | 60 (ramp) | 100 | (96-106) |
| | T = 3 months | 10 | 95 | (87-109) | 10 | 95 | (92-99) |
| | 25° C./60% RH | 15 | 99 | (91-113) | 15 | 97 | (93-102) |
| | | 30 | 101 | (93-113) | 30 | 98 | (94-104) |
| | | 45 | 101 | (93-112) | 45 | 100 | (95-105) |
| | | 60 (ramp) | 103 | (95-113) | 60 (ramp) | 103 | (99-109) |
| | T = 3 months | 10 | 71 | (64-82) | 10 | 84 | (78-87) |
| | 40° C./75% RH | 15 | 80 | (73-86) | 15 | 86 | (81-90) |
| | | 30 | 85 | (74-93) | 30 | 86 | (81-91) |
| | | 45 | 87 | (77-94) | 45 | 88 | (82-91) |
| | | 60 (ramp) | 101 | (100-103) | 60 (ramp) | 100 | (96-109) |
| | T = 6 months | 10 | 82 | (80-86) | 10 | 51 | (30-61) |
| | 25° C./60% RH | 15 | 90 | (88-93) | 15 | 62 | (53-70) |
| | | 30 | 91 | (88-94) | 30 | 68 | (62-74) |
| | | 45 | 90 | (89-92) | 45 | 70 | (66-76) |
| | | 60 (ramp) | 91 | (88-93) | 60 (ramp) | 85 | (83-88) |
| Formulation | | Amphotericin B with Gelucire | | | Amphotericin B with Gelucire | | |
| | | 44/14-Peceol-TPGS (10 mg-10 mg-1 mg), | | | 44/14-Peceol-TPGS (10 mg-10 mg-1 mg), | | |
| | | mannitol/microcrystalline | | | silicified microcrystalline | | |
| | | cellulose oral dose formulation in | | | cellulose/croscarmellose oral | | |
| | | hard shell capsule | | | dose formulation in hard shell | | |
| | | | | | capsule | | |
| Dose strength | | 100 mg/capsule | | | | | |
| | T = 6 months | 10 | 24 | (22-27) | 10 | 81 | (80-84) |
| | 40° C./75% RH | 15 | 61 | (56-67) | 15 | 86 | (85-88) |
| | | 30 | 69 | (65-75) | 30 | 87 | (85-89) |
| | | 45 | 79 | (68-87) | 45 | 87 | (86-89) |
| | | 60 (ramp) | 90 | (88-93) | 60 (ramp) | 90 | (86-95) |

### Formulation 5A

Another stability study was initiated for Formulation 5A. The capsules were packaged as per Formulation 1A and Formulation 2A and store under the same conditions.

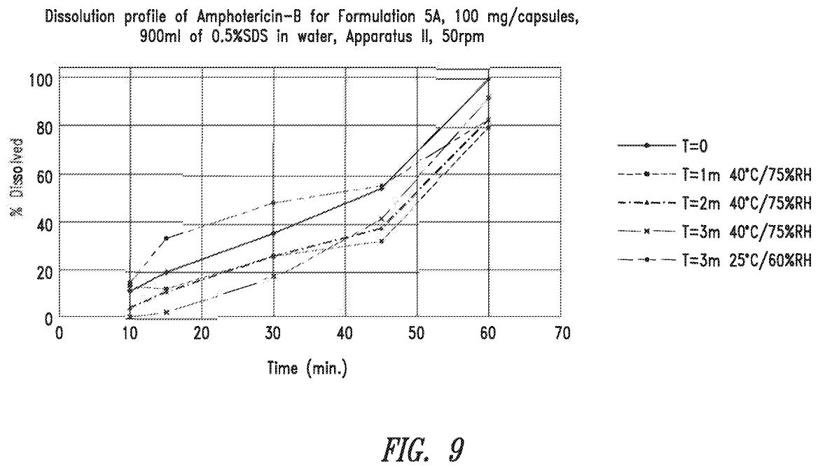

An Amphotericin B/TPGS/Peceol/Gelucire 44/14 semi-solid lipid based formulation in hard shell capsule was prepared (Formulation 5A) and stored under ICH controlled stability conditions. After 3 months of storage, the formulation remained stable with no loss of potency (Table 21) and no increases in related substances (Table 22). The dissolution profile however decreased (Table 23 and FIG. 9). As indicated before, this behavior could be a result a recrystallization or aggregation of the AmpB.

TABLE 21
Stability Results for Formulation 5A.

| Sample | | Formulation 5A |
|---|---|---|
| Formulation | | 11.1% drug load |
| Dose strength | | 100 mg AmpB/caps |
| Appearance | T = 0 | Yellow paste in white capsule |
| | T = 1 month | Yellow paste in white capsule (a clear liquid is |
| | 40° C./75% RH | separated in the capsule) |
| | T = 2 months | Yellow paste in white capsule (a clear liquid is |
| | 40° C./75% RH | separated in the capsule and a liquid is observed |
| | | exuding the capsule) |
| | T = 3 months | Yellow paste in white capsule |
| | 25° C./60% RH | |
| | T = 3 months | Yellow paste in white capsule (a liquid is observed |
| | 40° C./75% RH | exuding the capsule) |
| Water content | T = 0 | 1.9% |
| | T = 1 month | 5.1% |
| | 40° C./75% RH | |
| | T = 2 months | 2.3% |
| | 40° C./75% RH | |
| | T = 3 months | 2.0% |
| | 25° C./60% RH | |
| | T = 3 months | 2.2% |
| | 40° C./75% RH | |
| Assay | T = 0 | 95.1% |
| % Label claimed | | (n = 6: 94.9, 95.8, |
| Corealis -26801-AD-01 | | 93.7, 96.2, 95.4, 94.6) |
| Rev R&D 06 | T = 1 month | 97.0% |
| | 40° C./75% RH | (n = 6: 97.4, 97.6, |
| | | 98.1, 96.6, 95.0, 97.4) |
| | T = 2 months | 95.2% |
| | 40° C./75% RH | (n = 6: 96.7, 97.1, |
| | | 93.9, 96.3, 94.2, 92.7) |
| | T = 3 months | 99.1%^{A} |
| | 25° C./60% RH | (n = 5: 96.5, 97.5, |
| | | 101.3, 100.5, 99.8) |
| | T = 3 months | 95.3%^{A} |
| | 40° C./75% RH | (n = 5: 98.6, 95.6, |
| | | 93.6. 97.2. 91.5) |

^{A}Not enough sample units available for n = 6

TABLE 22
Related Substances Stability Results for Formulation 5A

| Sample | | Formulation 5A | | | |
|---|---|---|---|---|---|
| Dose Strength | | 100 mg/capsule | | | |
| | | | | API | |
| | | | | (Corealis lot | |
| | | Formulation 5A | | C00729) | |
| | | RRT | % area | RRT | % area |
| Related Substances | T = 0 | 0.15 | 0.24 | 0.15 | 0.15 |
| Corealis -26801-AD-01 | | 0.20 | 0.12 | 0.31 | 0.80 |
| Rev R&D 04 | | 0.31 | 0.83 | 0.50 | 0.14 |
| (% area) | | 0.50 | 0.13 | 0.63 | 0.58 |
| | | 0.63 | 0.23 | 0.69 | 0.14 |
| | | 0.69 | 0.14 | 0.74 | 0.63 |
| | | 0.74 | 0.63 | 0.79 | 0.15 |
| | | 0.79 | 0.14 | 0.83 | 0.95 |
| | | 0.83 | 0.87 | 1.13 | 0.59 |
| | | 1.13 | 0.56 | 1.19 | 0.71 |
| | | 1.19 | 0.64 | 1.22 | 0.16 |
| | | 1.22 | 0.21 | 1.27 | 0.24 |
| | | 1.27 | 0.21 | 1.81 | 1.02 |
| | | 1.82 | 1.02 | 2.16 | 1.37 |
| | | 2.16 | 1.23 | 2.25 | 0.12 |
| | | 2.25 | 0.12 | Total | 7.75 |
| | | Total | 7.32 | | |
| | T = 1 month | 0.13 | 0.18 | 0.13 | 0.12 |
| | 40° C./75% RH | 0.18 | 0.15 | 0.30 | 0.70 |
| | | 0.30 | 0.72 | 0.49 | 0.13 |
| | | 0.49 | 0.13 | 0.63 | 0.25 |
| | | 0.69 | 0.13 | 0.69 | 0.15 |
| | | 0.73 | 0.55 | 0.73 | 0.61 |
| | | 0.78 | 0.24 | 0.78 | 0.23 |
| | | 0.82 | 0.90 | 0.82 | 0.85 |
| | | 1.11 | 0.74 | 1.11 | 0.53 |
| | | 1.15 | 0.48 | 1.15 | 0.60 |
| | | 1.19 | 0.34 | 1.19 | 0.19 |
| | | 1.26 | 0.12 | 1.26 | 0.15 |
| | | 1.79 | 1.02 | 1.79 | 1.03 |
| | | 1.99 | 0.19 | 2.06 | 1.36 |
| | | 2.06 | 0.78 | 2.19 | 0.15 |
| | | 2.19 | 0.13 | Total | 7.04 |
| | | 3.83 | 0.11 | | |
| | | Total | 6.91 | | |
| | T = 2 months | 0.13 | 0.21 | 0.13 | 0.11 |
| | 40° C./75% RH | 0.18 | 0.18 | 0.29 | 0.76 |
| | | 0.29 | 0.75 | 0.48 | 0.13 |
| | | 0.49 | 0.13 | 0.62 | 0.37 |
| | | 0.69 | 0.15 | 0.68 | 0.14 |
| | | 0.73 | 0.34^{B} | 0.73 | 0.29 |
| | | 0.74 | 0.22^{B} | 0.74 | 0.27 |
| | | 0.79 | 0.15 | 0.78 | 0.14 |
| | | 0.83 | 0.53 | 0.82 | 0.53 |
| | | 0.84 | 0.35 | 0.85 | 0.30 |
| | | 1.14 | 0.99 | 1.16 | 0.67 |
| | | 1.22 | 0.61 | 1.21 | 0.69 |
| | | 1.25 | 0.48 | 1.26 | 0.31 |
| | | 1.30 | 0.12 | 1.78 | 1.04 |
| | | 1.74 | 1.02 | 2.15 | 1.49 |
| | | 2.05 | 0.28 | Total | 7.27 |
| | | 2.10 | 0.77 | | |
| | | 3.04 | 0.11 | | |
| | | 3.73 | 0.17 | | |
| | | Total | 7.57 | | |
| | T = 3 months | 0.13 | 0.12 | 0.13 | 0.11 |
| | 25° C./60% RH | 0.18 | 0.14 | 0.29 | 0.83 |
| | | 0.29 | 0.85 | 0.49 | 0.14 |
| | | 0.49 | 0.14 | 0.62 | 0.47 |
| | | 0.69 | 0.15 | 0.69 | 0.14 |
| | | 0.74 | 0.56^{E} | 0.74 | 0.66 |
| | | 0.79 | 0.19 | 0.79 | 0.23 |
| | | 0.83 | 0.53 | 0.83 | 0.60 |
| | | 0.84 | 0.35 | 0.83 | 0.26 |
| | | 1.14 | 0.98 | 1.13 | 0.57 |
| | | 1.18 | 0.65 | 1.17 | 0.76 |
| | | 1.22 | 0.32 | 1.22 | 0.14 |
| | | 1.25 | 0.11 | 1.25 | 0.17 |
| | | 1.74 | 1.01 | 1.74 | 1.03 |
| | | 1.98 | 0.24 | 2.04 | 1.32 |
| | | 2.05 | 0.56 | 2.10 | 0.12 |
| | | 2.10 | 0.12 | Total | 7.55 |
| | | 2.86 | 0.15 | | |
| | | 3.73 | 0.18 | | |
| | | Total | 7.35 | | |
| | | RRT | | | |
| | | RRT | % area | | |
| | T = 3 months | 0.13 | 0.15 | | |
| | 40° C./75% RH | 0.18 | 0.12 | | |
| | | 0.29 | 0.85 | | |
| | | 0.49 | 0.15 | | |
| | | 0.63 | 0.12 | | |
| | | 0.69 | 0.15 | | |
| | | 0.74 | 0.63^{E} | | |
| | | 0.79 | 0.20 | | |
| | | 0.83 | 0.53 | | |
| | | 0.84 | 0.26 | | |
| | | 1.14 | 0.75 | | |
| | | 1.18 | 0.68 | | |
| | | 1.22 | 0.28 | | |
| | | 1.25 | 0.14 | | |
| | | 1.74 | 1.01 | | |
| | | 1.98 | 0.13 | | |
| | | 2.05 | 0.92 | | |
| | | 2.10 | 0.13 | | |
| | | Total | 7.18 | | |

Note:
Peaks ≥0.10% reported; the reported impurity profile (% area) is equivalent in the API and sample solutions.
^{B}The two peaks observed at RRT 0.73 and RRT 0.74 were previously observed as one peak at RRT 0.73
^{E}The peak observed at RRT 0.74 is made of 2 coeluting peaks only observed separately on the analysis at the 2 m time point.

TABLE 23
Dissolution Profile Stability Results for Formula 5A

| Sample | | Formula 5A | | |
|---|---|---|---|---|
| Dose strength | | 100 mg/capsule | | |
| | | Time (min.) | % dissolved | Range |
| Dissolution 900 ml 0.5% | T = 0 | 10 | 12 | 2, 22 |
| SDS in Water paddles at | | 15 | 20 | 12, 28 |
| 50 rpm % dissolved | | 30 | 36 | 28, 44 |
| Corealis -26801-B-01 | | 45 | 55 | 52, 57 |
| Rev R&D 02 (T = 0, n = 2 | | 60 | 101 | 98, 103 |
| T = 1 m, 2 m, n = 3) | | (ramp) | | |
| (T = 3 m, n = 3) | | | | |
| | | Time (min.) | % dissolved | Range^{C} |
| | T = 1 month | 10 | 14 | 2, 24, 15 |
| | 40° C./75% RH | 15 | 13 | 5, 16, 17 |
| | | 30 | 27 | 20, 28, 32 |
| | | 45 | 33 | 30, 35, 35 |
| | | 60 | 81 | 81, 81, 80 |
| | | (ramp) | | |
| | T = 2 months | 10 | 5 | 1, 4, 11 |
| | 40° C./75% RH | 15 | 12 | 10, 10, 17 |
| | | 30 | 27 | 18, 21, 41 |
| | | 45 | 38 | 24, 34, 57 |
| | | 60 | 84 | 90, 83, 78 |
| | | (ramp) | | |
| | T = 3 months | 10 | 15 | 27, 7, 10 |
| | 25° C./60% RH | 15 | 34 | 52, 26, 24 |
| | | 30 | 49 | 63, 49, 35 |
| | | 45 | 56 | 70, 53, 44 |
| | | 60 | 84 | 92, 87, 74 |
| | | (ramp) | | |
| | | Time (min.) | % dissolved | Range^{C, D} |
| | T = 3 months | 10 | 1 | 1, 1, 2 |
| | 40° C./75% RH | 15 | 3 | 1, 3, 6 |
| | | 30 | 17 | 22, 16, 13 |
| | | 45 | 42 | 41, 49, 37 |
| | | 60 | 93 | 90, 94, 95 |
| | | (ramp) | | |

^{C}After 45 minutes at 50 rpm, pieces of the capsule and it contents remain in the sinker.
^{D}The capsules disintegrated slowly compared to the samples at 40° C./75% RH (3 months).

### Example 8

### Pharmacokinetic Studies in Humans

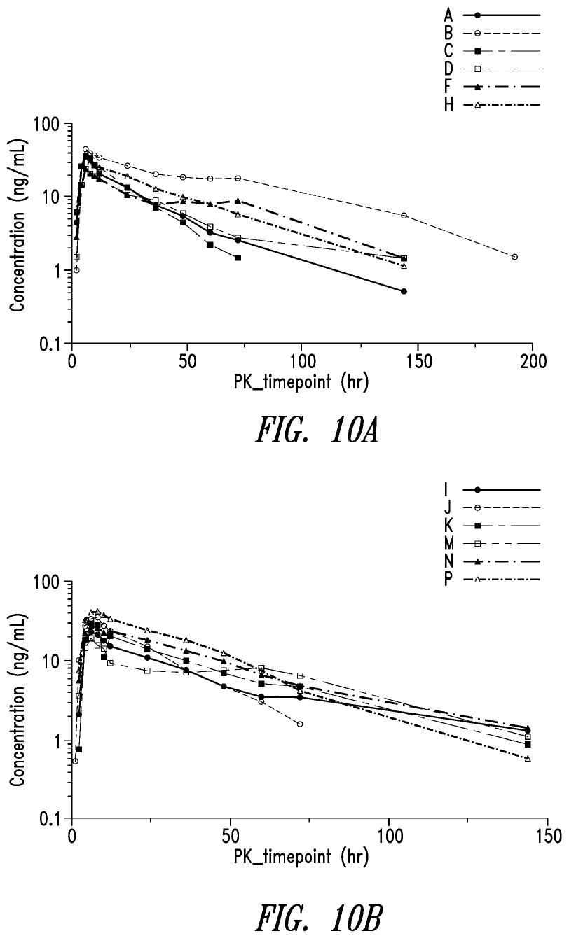

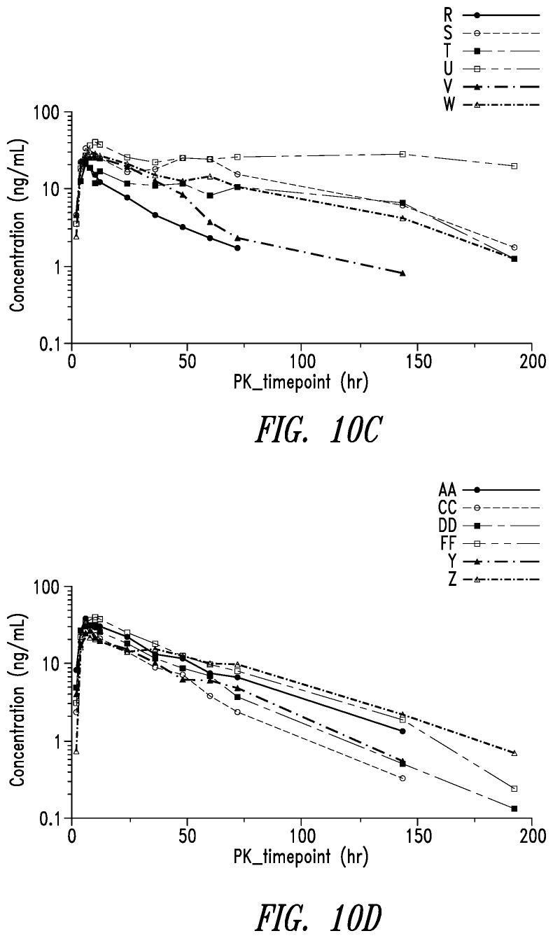

A phase 1, randomized, double-blind, placebo-controlled, single dose escalation study was performed to evaluate the pharmacokinetic parameters after administration of a single dose of Formula 1A. 100 mg (FIG. 10A), 200 mg (FIG. 10B), 400 mg (FIG. 10C) and 800 mg (FIG. 10D) doses were investigated in this study.

For each dose, AmpB or a placebo was administered, and blood samples were obtained pre-dose, and post-dosing at 0.5 hr, 1 hr, 2 hr, 3 hr, 4 hr, 6 hr, 8 hr, 10 hr, 12 hr, 24 hr, 36 hr, 48 hr, 60 hr, 72 hr, 144 hr, and 192 hr. Pharmacokinetic analysis was performed using Phoenix™ WinNonlin® (Pharsight Corporation). Doses and sampling times were entered as provided by the sponsor. Dose normalised parameters, and descriptive statistics were calculated using Phoenix™ WinNonlin®.

The following pharmacokinetic parameters were measured:
Tmax: Time to reach the maximum observed concentration.
Cmax: Maximum observed concentration.
AUC0-t: Area under the concentration-time curve from hour 0 to the last measurable concentration (72 hr), estimated by the linear trapezoidal rule.
AUC0-inf: Area under the concentration-time curve from hour 0 to infinity, calculated as follows:

AUC0-inf=AUC0-t+Ct/Kel

Where Ct is the last measurable concentration and Kel is the elimination rate constant estimated using log-linear regression during the terminal elimination phase.
t½: Elimination half-life, determined by ln(2)/Kel
CL/f: Observed Systemic clearance, calculated as dose/AUC
Vz: Observed Volume of distribution during the terminal phase, calculated as CL/Kel

TABLE 24
Pharmacokinetic Parameters for Amphotericin B in Human Plasma
for Patients Dosed with 100 mg of Amphotericin B or Placebo.

| | Tmax | Cmax | Kel | AUCO-t | AUCO-inf | % AUCexp | V/F | CL/F | T½ |
|---|---|---|---|---|---|---|---|---|---|
| Subject_ID | (hr) | (ng/mL) | (1/hr) | (hr*ng/mL) | (hr*ng/mL) | (%) | (mL) | (mL/hr) | (hr) |
| A | 6.00 | 25.98 | 0.04 | 716.57 | 788.16 | 9.08 | 3545589.51 | 126877.64 | 19.37 |
| B | 6.00 | 43.57 | 0.01 | 1606.43 | 3042.77 | 47.20 | 2613781.85 | 32864.80 | 55.13 |
| C | 6.00 | 34.67 | 0.05 | 761.59 | 792.54 | 3.91 | 2624477.13 | 126176.85 | 14.42 |
| D | 6.00 | 22.66 | 0.03 | 635.82 | 723.90 | 12.17 | 4503070.74 | 138140.40 | 22.60 |
| F | 6.00 | 24.41 | 0.01 | 757.71 | 1362.29 | 44.38 | 5016960.70 | 73405.78 | 47.37 |
| H | 6.00 | 30.05 | 0.02 | 996.95 | 1267.06 | 21.32 | 3637239.70 | 78922.85 | 31.94 |
| N | 6 | 6 | 6 | 6 | 6 | 6 | 6 | 6 | 6 |
| Mean | 6.00 | 30.22 | 0.03 | 912.51 | 1329.45 | 23.01 | 3656853.27 | 96064.72 | 31.80 |
| SD | 0.00 | 7.84 | 0.01 | 360.65 | 881.86 | 18.55 | 972886.33 | 41056.16 | 16.30 |
| Min | 6.00 | 22.66 | 0.01 | 635.82 | 723.90 | 3.91 | 2613781.85 | 32864.80 | 14.42 |
| Median | 6.00 | 28.02 | 0.03 | 759.65 | 1029.80 | 16.74 | 3591414.60 | 102549.85 | 27.27 |
| Max | 6.00 | 43.57 | 0.05 | 1606.43 | 3042.77 | 47.20 | 5016960.70 | 138140.40 | 55.13 |
| CV % | 0.00 | 25.92 | 49.90 | 39.52 | 66.33 | 80.64 | 26.60 | 42.74 | 51.24 |
| Geometric | 6.00 | 29.45 | 0.02 | 865.75 | 1155.07 | 16.37 | 3549117.10 | 86574.82 | 28.42 |
| Mean | | | | | | | | | |

Two samples recorded analyte concentrations below the level of quantification (BQL) for all timepoints (Blind Subject ID's E and G). These were excluded from pharmacokinetic parameter reporting.

TABLE 25
Pharmacokinetic Parameters for Amphotericin B in Human Plasma
for Patients Dosed with 200 mg of Amphotericin B or Placebo.

| | Tmax | Cmax | Kel | AUCO-t | AUCO-inf | % AUCexp | V/F | CL/F | T½ |
|---|---|---|---|---|---|---|---|---|---|
| Subject_ID | (hr) | (ng/mL) | (1/hr) | (hr*ng/mL) | (hr*ng/mL) | (%) | (mL) | (mL/hr) | (hr) |
| I | 6.00 | 23.41 | 0.03 | 614.86 | 737.08 | 16.58 | 9591266.45 | 271340.46 | 24.50 |
| 3 | 8.00 | 36.31 | 0.05 | 825.30 | 860.11 | 4.05 | 5119460.58 | 232527.11 | 15.26 |
| K | 6.00 | 29.90 | 0.03 | 782.74 | 976.64 | 19.85 | 8132757.78 | 204784.12 | 27.53 |
| M | 6.00 | 18.77 | 0.01 | 596.14 | 1241.16 | 51.97 | 15999011.64 | 161138.94 | 68.82 |
| N | 6.00 | 27.35 | 0.03 | 973.27 | 1146.01 | 15.07 | 6215853.68 | 174518.59 | 24.69 |
| P | 6.00 | 42.46 | 0.05 | 1344.94 | 1438.52 | 6.50 | 3073693.76 | 139032.26 | 15.32 |
| N | 6 | 6 | 6 | 6 | 6 | 6 | 6 | 6 | 6 |
| Mean | 6.33 | 29.70 | 0.03 | 856.21 | 1066.59 | 19.01 | 8022007.31 | 197223.58 | 29.35 |
| SD | 0.82 | 8.61 | 0.01 | 277.42 | 258.62 | 17.26 | 4522835.69 | 48991.14 | 20.01 |
| Min | 6.00 | 18.77 | 0.01 | 596.14 | 737.08 | 4.05 | 3073693.76 | 139032.26 | 15.26 |
| Median | 6.00 | 28.62 | 0.03 | 804.02 | 1061.32 | 15.83 | 7174305.73 | 189651.36 | 24.59 |
| Max | 8.00 | 42.46 | 0.05 | 1344.94 | 1438.52 | 51.97 | 15999011.64 | 271340.46 | 68.82 |
| CV % | 12.89 | 29.01 | 44.06 | 32.40 | 24.25 | 90.79 | 56.38 | 24.84 | 68.16 |
| Geometric | 6.29 | 28.66 | 0.03 | 822.65 | 1040.21 | 13.76 | 7043125.41 | 192268.01 | 25.39 |
| Mean | | | | | | | | | |

Two samples recorded analyte concentrations below the level of quantification (BQL) for all timepoints (Blind Subject ID's O and L). These were excluded from pharmacokinetic parameter reporting.

TABLE 26
Pharmacokinetic Parameters for Amphotericin B in Human Plasma
for Patients Dosed with 400 mg of Amphotericin B or Placebo.

| | Tmax | Cmax | Kel | AUCO-t | AUCO-inf | % AUCexp | V/F | CL/F | T½ |
|---|---|---|---|---|---|---|---|---|---|
| Subject_ID | (hr) | (ng/mL) | (1/hr) | (hr*ng/mL) | (hr*ng/mL) | (%) | (mL) | (mL/hr) | (hr) |
| R | 6.00 | 21.86 | 0.03 | 461.79 | 520.98 | 11.36 | 26779550.10 | 767787.87 | 24.18 |
| S | 6.00 | 34.26 | 0.02 | 1550.60 | 2265.43 | 31.55 | 8154849.30 | 176567.28 | 32.01 |
| T | 6.00 | 20.15 | 0.01 | 845.03 | 2139.96 | 60.51 | 22728341.15 | 186919.05 | 84.28 |
| U | 10.00 | 41.12 | 0.00 | 1856.79 | 7138.99 | 73.99 | 11482617.37 | 56030.30 | 142.05 |
| V | 6.00 | 26.38 | 0.05 | 936.40 | 981.05 | 4.55 | 8037364.81 | 407726.35 | 13.66 |
| W | 10.00 | 30.49 | 0.02 | 1242.03 | 1918.71 | 35.27 | 13851792.91 | 208473.83 | 46.06 |
| N | 6 | 6 | 6 | 6 | 6 | 6 | 6 | 6 | 6 |
| Mean | 7.33 | 29.04 | 0.02 | 1148.77 | 2494.19 | 36.21 | 15172419.27 | 300584.11 | 57.04 |
| SD | 2.07 | 7.91 | 0.02 | 506.12 | 2377.66 | 27.06 | 7840043.68 | 255479.79 | 48.33 |
| Min | 6.00 | 20.15 | 0.00 | 461.79 | 520.98 | 4.55 | 8037364.81 | 56030.30 | 13.66 |
| Median | 6.00 | 28.44 | 0.02 | 1089.21 | 2029.33 | 33.41 | 12667205.14 | 197696.44 | 39.03 |
| Max | 10.00 | 41.12 | 0.05 | 1856.79 | 7138.99 | 73.99 | 26779550.10 | 767787.87 | 142.05 |
| CV % | 28.17 | 27.23 | 77.70 | 44.06 | 95.33 | 74.73 | 51.67 | 84.99 | 84.72 |
| Geometric | 7.11 | 28.16 | 0.02 | 1045.59 | 1799.36 | 25.23 | 13606337.94 | 222300.98 | 42.43 |
| Mean | | | | | | | | | |

Two samples recorded analyte concentrations below the level of quantification (BQL) for all timepoints (Blind Subject ID's Q and X). These were excluded from pharmacokinetic parameter reporting.

TABLE 27
Pharmacokinetic Parameters for Amphotericin B in Human Plasma
for Patients Dosed with 800 mg of Amphotericin B or Placebo.

| Deidentified | Dosage | Pre- | Nominal hour (hr) | | | | | | | |
|---|---|---|---|---|---|---|---|---|---|---|
| Subject ID | (mg) | dose | 0.5 | 1 | 2 | 4 | 6 | 8 | 10 | 12 |

Amphotericin B concentration (ng/mL)

| | | | | | | | | | | |
|---|---|---|---|---|---|---|---|---|---|---|
| Y | 800 mg & | BQL | BQL | BQL | 4.175 | 20.176 | 31.375 | 22.816 | 22.226 | 21.390 |
| Z | placebo | BQL | BQL | 0.791 | 7.683 | 16.397 | 29.902 | 22.535 | 21.595 | 21.001 |
| AA | | BQL | BQL | BQL | 8.234 | 28.318 | 40.067 | 31.543 | 29.990 | 31.865 |
| BB | | BQL | BQL | BQL | BQL | BQL | BQL | BQL | BQL | BQL |
| CC | | BQL | BQL | BQL | 2.414 | 18.814 | 30.628 | 30.838 | 25.996 | 22.810 |
| DD | | BQL | BQL | BQL | 5.164 | 17.942 | 24.140 | 27.279 | 32.914 | 27.866 |
| EE | | BQL | BQL | BQL | BQL | BQL | BQL | BQL | BQL | BQL |
| FF | | BQL | BQL | BQL | 3.206 | 23.486 | 33.962 | 39.722 | 42.805 | 40.021 |

| Deidentified | Nominal hour (hr) | | | | | | |
|---|---|---|---|---|---|---|---|
| Subject ID | 24 | 36 | 48 | 63 | 72 | 144 | 192 |

Amphotericin B concentration (ng/mL)

| | | | | | | | |
|---|---|---|---|---|---|---|---|
| Y | 16.218 | 10.661 | 6.472 | 6.398 | 5.044 | 0.539 | BQL |
| Z | 14.674 | 16.074 | 12.880 | 10.642 | 10.165 | 2.254 | 0.726 |
| AA | 23.607 | 13.715 | 12.291 | 7.859 | 6.793 | 1.380 | BQL |
| BB | BQL | BQL | BQL | BQL | BQL | BQL | BQL |
| CC | 14.816 | 9.365 | 7.395 | 3.935 | 2.412 | 0.329 | BQL |
| DD | 19.124 | 12.504 | 8.947 | 6.865 | 3.808 | 0.518 | 0.132 |
| EE | BQL | BQL | BQL | BQL | BQL | BQL | BQL |
| FF | 26.064 | 19.218 | 13.166 | 9.972 | 8.297 | 1.905 | 0.243 |

Two samples recorded analyte concentrations below the level of quantification (BQL) for all timepoints (Blind Subject ID's BB and EE). These were excluded from pharmacokinetic parameter reporting

1. A solid dosage form comprising:
a) amphotericin B in an amount from about 100 to about 800 mg; and
b) at least one lipophilic component,
wherein the amphoteric B and the at least one lipophilic component are coated on a solid carrier, and
wherein the solid dosage form provides at least one of the following pharmacokinetic parameters:
i) an average maximum blood plasma concentration (Cmax) of amphotericin B within about 80%-125% of the range of from about 21.09 ng/mL to about 42.07 ng/mL, after a single dose of about 100-800 mg of amphotericin B;
ii) an average time to Cₘₐₓ (Tₘₐₓ) within about 80%-125% of the range of from about 5.25 hr to about 9.66 hr after a single dose of about 100-800 mg of amphotericin B;
iii) an average AUC₀₋ₜ within about 80%-125% of the range of from about 510.00 hr*ng/mL to about 4779.45.89 hr*ng/mL after a single dose of about 100-800 mg of amphotericin B; or
iv) an average AUC_{0-inf} within about 80%-125% of the range of from about 509.84 18366.24 hr*ng/mL to about 18366.24 hr*ng/mL after a single dose of about 100 to about 400 mg of amphotericin B.

2. The solid dosage form of claim 1, comprising about 100 mg, about 200 mg, about 300 mg, about 400 mg, about 500 mg, about 600 mg, about 700 mg, or about 800 mg of amphotericin B.

3. The solid dosage form of claim 1 comprising about 100 mg of amphotericin B, wherein the solid dosage form provides at least one of the following pharmacokinetic parameters:
i) an average Cₘₐₓ within about 80%-125% of the range of about 30.22±7.84 ng/mL, after a single dose of about 100 mg of amphotericin B;
ii) an average Tₘₐₓ within about 80%-125% of the range of about 6 hr after a single dose of about 100 mg of amphotericin B;
iii) an average AUC₀₋ₜ within about 80%-125% of the range of about 1228.86±710.86 hr*ng/mL after a single dose of about 100 mg of amphotericin B; or
iv) an average AUC_{0-inf} within about 80%-125% of the range of about 1083.35±269.57 hr*ng/mL after a single dose of about 100 mg of amphotericin B.

4. The solid dosage form of claim 1 comprising about 200 mg of amphotericin B, wherein the solid dosage form provides at least one of the following pharmacokinetic parameters:
i) an average Cₘₐₓ within about 80%-125% of the range of about 29.70±8.61 ng/mL, after a single dose of about 200 mg of amphotericin B;
ii) an average Tₘₐₓ within about 80%-125% of the range of about 6.33±0.82 hr after a single dose of about 200 mg of amphotericin B;
iii) an average AUC₀₋ₜ within about 80%-125% of the range of about 1031.10±281.31 hr*ng/mL after a single dose of about 200 mg of amphotericin B; or
iv) an average AUC_{0-inf} within about 80%-125% of the range of about 1083.35±269.57 hr*ng/mL after a single dose of about 200 mg of amphotericin B.

5. The solid dosage form of claim 1 comprising about 400 mg of amphotericin B, wherein the solid dosage form provides at least one of the following pharmacokinetic parameters:
i) an average Cₘₐₓ within about 80%-125% of the range of about 36.65±5.42 ng/mL, after a single dose of about 400 mg of amphotericin B;
ii) an average Tₘₐₓ within about 80%-125% of the range of about 7.69±1.97 hr after a single dose of about 400 mg of amphotericin B;
iii) an average AUC₀₋ₜ within about 80%-125% of the range of about 2093.35±1583.16 hr*ng/mL after a single dose of about 400 mg of amphotericin B; or
iv) an average AUC_{0-inf} within about 80%-125% of the range of about 4385.39±6887.45 hr*ng/mL after a single dose of about 400 mg of amphotericin B.

6. The solid dosage form of claim 1 comprising about 800 mg of amphotericin B, wherein the solid dosage form provides at least one of the following pharmacokinetic parameters:
i) an average Cₘₐₓ within about 80%-125% of the range of about 29.04±7.91 ng/mL, after a single dose of 800 mg of amphotericin B;
ii) an average Tₘₐₓ within about 80%-125% of the range of about 7.33±2.07 hr after a single dose of 800 mg of amphotericin B;
iii) an average AUC₀₋ₜ within about 80%-125% of the range of about 1350.16±355.58 hr*ng/mL after a single dose of about 800 mg of amphotericin B; or
iv) an average AUG-_{0-inf} within about 80%-125% of the range of about 1373.76±363.07 hr*ng/mL after a single dose of about 800 mg of amphotericin B.

7. The solid dosage form of any of claims 1-6, wherein the the solid dosage form provides at least two of the pharmacokinetic parameters.

8. The solid dosage form of any of claims 1-7, wherein the the solid dosage form provides at least three of the pharmacokinetic parameters.

9. The solid dosage form of any of claims 1-8, wherein the the solid dosage form provides all four of the pharmacokinetic parameters.

10. A method of treating a disease in a subject in need thereof, comprising administering a solid dosage form comprising:
a) about 100-800 mg of amphotericin B; and
b) at least one lipophilic component,
wherein the amphoteric B and the at least one lipophilic component are coated on a solid carrier, and
wherein after administration, the patient has at least one of the following pharmacokinetic parameters:
i) an average Cₘₐₓ of amphotericin B within about 80%-125% of the range of from about 21.09 ng/mL to about 42.07 ng/mL;
ii) an average mean time to Tₘₐₓ within about 80%-125% of the range of from about 5.25 hr to about 9.66 hr;
iii) an average AUC₀₋ₜ within about 80%-125% of the range of from about 551.86 hr*ng/mL to about 1654.89 hr*ng/mL; or
iv) an average AUG_{0-inf} within about 80%-125% of the range of about 18366.24 hr*ng/mL to about 18366.24 hr*ng/mL after a single dose of about 100-800 mg of amphotericin B.

11. The method of claim 10, wherein the solid dosage form comprises about 100 mg of amphotericin B in the solid dosage form, and the patient has at least one of the following pharmacokinetic parameters after administering a single dose of about 100 mg of amphotericin B:
i) an average Cₘₐₓ within about 80%-125% of the range of about 30.22±7.84 ng/mL;
ii) an average Tₘₐₓ within about 80%-125% of about 6 hr;
iii) an average AUC₀₋ₜ within about 80%-125% of the range of about 1228.86±710.86 hr*ng/mL; or
iv) an average AUC_{0-inf} within about 80%-125% of the range of about 1083.35±269.57 hr*ng/mL.

12. The method of claim 10, wherein the solid dosage form comprises about 200 mg of amphotericin B, and the patient has at least one of the following pharmacokinetic parameters, after a single dose of about 200 mg of amphotericin B:
i) an average Cₘₐₓ within about 80%-125% of the range of about 29.70±8.61 ng/mL, after a single dose of about 200 mg of amphotericin B;
ii) an average Tₘₐₓ within about 80%-125% of the range of about 6.33±0.82 hr after a single dose of about 200 mg of amphotericin B;
iii) an average AUC₀₋ₜ within about 80%-125% of the range of about 1031.10±281.31 hr*ng/mL after a single dose of about 200 mg of amphotericin B; or
iv) an average AUC_{0-inf} within about 80%-125% of the range of about 1083.35±269.57 hr*ng/mL after a single dose of about 200 mg of amphotericin B.

13. The method claim 10, wherein the solid dosage form comprises about 400 mg of amphotericin B, and the solid dosage form provides at least one of the following pharmacokinetic parameters after a single dose of about 400 mg of amphotericin B:
i) an average Cₘₐₓ within 80%-125% of the range of about 36.65±5.42 ng/mL;
ii) an average Tₘₐₓ within about 80%-125% of the range of about 7.69±1.97 hr;
iii) an average AUC₀₋ₜ within about 80%-125% of the range of about 2093.35±1583.16 hr*ng/mL; or
iv) an average AUC_{0-inf} within 80%-125% of the range of 4385.39±6887.45 hr*ng/mL.

14. The method claim 10, wherein the solid dosage form comprises about 800 mg of amphotericin B, and the solid dosage form provides at least one of the following pharmacokinetic parameters:
i) an average Cₘₐₓ within about 80%-125% of the range of about 29.04±7.91 ng/mL, after a single dose of 800 mg of amphotericin B;
ii) an average Tₘₐₓ within about 80%-125% of the range of about 7.33±2.07 hr after a single dose of 800 mg of amphotericin B;
iii) an average AUC₀₋ₜ within about 80%-125% of the range of about 1350.16±355.58 hr*ng/mL after a single dose of about 800 mg of amphotericin B; or iv) an average AUC_{0-inf} within about 80%-125% of the range of about 1373.76±363.07 hr*ng/mL after a single dose of about 800 mg of amphotericin B.

15. The method of any of claims 10-14, wherein the subject is human.

16. The method of claims 10-15, wherein the subject is treated for an infectious disease.

17. The method of claim 16, wherein the infectious disease is a fungal infection, human immunodeficiency virus (HIV), or a parasitic infection

18. The method of claim 17, wherein the fungal infection is aspergillosis, blastomycosis, candidiasis, coccidioidomycosis, crytococcosis, histoplasmosis, mucormycosis, paracoccidioidomycosis, or sporotrichosis.

19. The method of claim 18, wherein the parasitic infection is visceral leishmaniasis, cutaneous leishmaniasis, mucocutaneous leishmaniasis, or Chagas disease.

20. The method of claim 16, wherein the infectious disease is leishmaniasis

21. The method of claim 16, wherein the infectious disease is Febrile neutropenia.

22. A method of treating a disease in a subject in need thereof, comprising administering a solid dosage form comprising amphotericin B in an amount in the range of from 100-800 mg, wherein after administration, the patient has an average AUC₀₋ₜ within 80%-125% of the range of from about 551.86 hr*ng/mL to about 1654.89 hr*ng/mL, or an average AUC_{0-inf} within 80%-125% of the range of 18366.24 hr*ng/mL to about 18366.24 hr*ng/mL.

23. The method of claim 22, wherein about 100 mg of amphotericin B is administered, and the patient has an average AUC₀₋ₜ within about 80%-125% of the range of about 1228.86±710.86 hr*ng/mL of amphotericin B, or an average AUC_{0-inf} within about 80%-125% of the range of about 1083.35±269.57 hr*ng/mL of amphotericin B.

24. The method of claim 22, wherein about 200 mg of amphotericin B is administered, and the patient has an average AUC₀₋ₜ within about 80%-125% of the range of about 1031.10±281.31 hr*ng/mL of amphotericin B, or an average AUC_{0-inf} within about 80%-125% of the range of about 1083.35±269.57 hr*ng/mL after a single dose of about 200 mg of amphotericin B.

25. The method of claim 22, wherein about 400 mg of amphotericin B is administered, and the patient has an average AUC₀₋ₜ within about 80%-125% of the range of about 2093.35±1583.16 hr*ng/mL of amphotericin B, or an average AUC_{0-inf} within 80%-125% of the range of 4385.39±6887.45 hr*ng/mL of amphotericin B.

26. The method of claim 22, wherein about 800 mg of amphotericin B is administered, and the the patient has an average AUC₀₋ₜ within about 80%-125% of the range of about 1350.16±355.58 hr*ng/mL of amphotericin B, or an average AUC_{0-inf} within about 80%-125% of the range of about 1373.76±363.07 hr*ng/mL after a single dose of about 800 mg of amphotericin B.

27. The method of any of claims 22-26, wherein the subject is human.

28. The method of claims 22-27, wherein the subject is treated for an infectious disease.

29. The method of claim 28, wherein the infectious disease is a fungal infection, human immunodeficiency virus (HIV), or a parasitic infection

30. The method of claim 29, wherein the fungal infection is aspergillosis, blastomycosis, candidiasis, coccidioidomycosis, crytococcosis, histoplasmosis, mucormycosis, paracoccidioidomycosis, or sporotrichosis.

31. The method of claim 29, wherein the parasitic infection is visceral leishmaniasis, cutaneous leishmaniasis, mucocutaneous leishmaniasis, or Chagas disease.

32. The method of claim 28, wherein the infectious disease is leishmaniasis

33. The method of claim 28, wherein the infectious disease is Febrile neutropenia.

34. The solid dosage form of any of claims 1-9, wherein the weight ratio amphotericin B to the at least lipophilic component is in the range of from about 10:1 to about 1:1.

35. The solid dosage form of any of claim 1-9 or 34, wherein the at least one lipophilic component comprises a first lipophilic component and a second lipophilic component.

36. The solid dosage form of claim 1-9, 34 or 35, wherein the weight ratio of the first lipophilic component and the second lipophilic component is about 75:25 to about 25:75.

37. The solid dosage form of claim 1-9, 34, 35, or 36, wherein the weight ratio of the first lipophilic component and the second lipophilic component is about 50:50.

38. The solid dosage form of claim 1-9 or 34-37, wherein the weight ratio of the first lipophilic component and the second lipophilic component is about 50:50.

39. The solid dosage form of any of claim 1-9 or 34-38, wherein the at least one lipophilic components comprises a fatty acid glycerol ester.

40. The solid dosage form of any of claim 1-9 or 34-39, wherein the at least one lipophilic complonent comprises a polyethylene oxide-containing fatty acid ester.

41. The solid dosage form of any of claim 1-9 or 34-40, wherein the at least one lipophilic components comprises mixture of a fatty acid glycerol ester and a polyethylene oxide-containing fatty acid ester.

42. The method of any one of claims 10-33, wherein the weight ratio amphotericin B to the at least lipophilic component is in the range of from about 10:1 to about 1:1.

43. The method of any one of claim 10-33 or 42, wherein the at least one lipophilic component comprises a first lipophilic component and a second lipophilic component.

44. The method of any one of claim 10-33, 42, or 43, wherein the at least one lipophilic component comprises a first lipophilic component and a second lipophilic component.

45. The method of any one of claim 10-33 or 42-44, wherein the weight ratio of the first lipophilic component and the second lipophilic component is about 75:25 to about 25:75.

46. The method of any one of claim 10-33 or 42-45, wherein the weight ratio of the first lipophilic component and the second lipophilic component is about 50:50.

47. The method of any one of claim 10-33 or 42-46, wherein the weight ratio of the first lipophilic component and the second lipophilic component is about 50:50.

48. The method of any one of claim 10-33 or 42-47, wherein the at least one lipophilic components comprises a fatty acid glycerol ester.

49. The method of any one of claim 10-33 or 42-48, wherein the at least one lipophilic complonent comprises a polyethylene oxide-containing fatty acid ester.

50. The method of any one of claim 10-33 or 42-49, wherein the at least one lipophilic components comprises mixture of a fatty acid glycerol ester and a polyethylene oxide-containing fatty acid ester.
